SEQUENCE LISTING (60 sequences)

SEQ ID NO: 1 - protein, 91 aa, Homo sapiens
MSFSGSDLCYMLELGEFEMKQETSERKPRGINSFLNGVCKSCQYEAPNCRENLQLCILAEISYYIRKTSIILDLIARNDIETLRSVLERWE

SEQ ID NO: 2 - DNA, 2326 bp, homo sapiens
agacagagcaaagccacaacgctttctctgctggattaaagacggcccacagaccagaacttccactatactacttaaaattacataggtggcttgtcaaattcaattgattagtattgtaaaaggaaaaagaagttccttcttacagcttggattcaacggtccaaaacaaaaatgcagctgccattaaagtcacagatgaacaaacttctacactgatttttaaaatcaagaataagggcagcaagtttctggattcactgaatcaacagacacaaaaagacatcattttacaacctcatttcaaaatgaagacttttacctggaccctaggtgtgctattcttcctactagtggacactggacattgcagaggtggacaattcaaaattaaaaaaataaaccagagaagataccctcgtgccacagatggtaaagaggaagcaaagaaatgtgcatacacattcctggtacctgaacaaagaataacagggccaatctgtgtcaacaccaaggggcaagatgcaagtaccattaaagacatgatcaccaggatggaccttgaaaacctgaaggatgtgctctccaggcagaagcgggagatagatgttctgcaactggtggtggatgtagatggaaacattgtgaatgaggtaaagctgctgagaaaggaaagccgtaacatgaactctcgtgttactcaactctatatgcaattattacatgagattatccgtaagagggataattcacttgaactttcccaactggaaaacaaaatcctcaatgtcaccacagaaatgttgaagatggcaacaagatacagggaactagaggtgaaatacgcttccttgactgatcttgtcaataaccaatctgtgatgatcactttgttggaagaacagtgcttgaggatattttcccgacaagacacccatgtgtctcccccacttgtccaggtggtgccacaacatattcctaacagccaacagtatactcctggtctgctgggaggtaacgagattcagagggatccaggttatcccagagatttaatgccaccacctgatctggcaacttctcccaccaaaagccctttcaagataccaccggtaactttcatcaatgaaggaccattcaaagactgtcagcaagcaaaagaagctgggcattcggtcagtgggatttatatgattaaacctgaaaacagcaatggaccaatgcagttatggtgtgaaaacagtttggaccctgggggttggactgttattcagaaaagaacagacggctctgtcaacttcttcagaaattgggaaaattataagaaagggtttggaaacattgacggagaatactggcttggactggaaaatatctatatgcttagcaatcaagataattacaagttattgattgaattagaagactggagtgataaaaaagtctatgcagaatacagcagctttcgtctggaacctgaaagtgaattctatagactgcgcctgggaacttaccagggaaatgcaggggattctatgatgtggcataatggtaaacaattcaccacactggacagagataaagatatgtatgcaggaaactgcgcccactttcataaaggaggctggtggtacaatgcctgtgcacattctaacctaaatggagtatggtacagaggaggccattacagaagcaagcaccaagatggaattttctgggccgaatacagaggcgggtcatactccttaagagcagttcagatgatgatcaagcctattgactgaagagagacactcgccaatttaaatgacacagaactttgtacttttcagctcttaaaaatgtaaatgttacatgtatattacttggcacaatttatttctacacagaaagtttttaaaatgaattttaccgtaactataaaagggaacctataaatgtagtttcatctgtcgtcaattactgcagaaaattatgtgtatccacaacctagttattttaaaaattatgttgactaaatacaaagtttgttttctaaaatgtaaatatttgccacaatgtaaagcaaatcttagctatattttaaatcataaataacatgttcaagatacttaacaatttatttaaaatctaagattgctctaacgtctagtgaaaaaaatatttttaaaatttcagccaaataatgcattttatttataaaaatacagacagaaaattagggagaaacctctagttttgccaatagaaaatgcttcttccattgaataaaagttatttcaaattgaaaaaaaaaaaaaaaaaaaaaaaaaaaaaaaaaaaaaaa

SEQ ID NO: 3 - protein, 191 aa, Homo sapiens
MQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 4 - DNA, 3572 bp, Homo sapiens
gcctttctggggcctgggggatcctcttgcactggtgggtggagagaagcgcctgcagccaaccagggtcaggctgtgctcacagtttcctctggcggcatgtaaaggctccacaaaggagttgggagttcaaatgaggctgctgcggacggcctgaggatggaccccaagccctggacctgccgagcgtggcactgaggcagcggctgacgctactgtgagggaaagaaggttgtgagcagccccgcaggacccctggccagccctggccccagcctctgccggagccctctgtggaggcagagccagtggagcccagtgaggcagggctgcttggcagccaccggcctgcaactcaggaacccctccagaggccatggacaggctgccccgctgacggccagggtgaagcatgtgaggagccgccccggagccaagcaggagggaagaggctttcatagattctattcacaaagaataaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacgctgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcctgacctctcgtggccattgccaggagcccaccctggtcacgctggccacagcacaaagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctctacaataaaaacacaactgcaaagtaccttcataatatacatgtgtatgagcctcccttgtgcacgtatgtgtataccacatatatatgcatttagatatacatcacatgtgatatatctagatccatatataggtttgccttagatacctaaatacacatatattcagttctcagatgttgaagctgtcaccagcagctttgctcttaggagaaaagcatttcattagtgttgtattacttgagtctaagggtagatcacagactgtgtggtctcaactgaaaggatcacccttggcatctgtgtgcctggattcttccagaatgtctacaatgctaatctctcacatagaggttcccagcttcttaagaaccccttttggcacctaatcaaatttcaaaatccctccccccacattttcatacttttccccattctcaggacttttcaccatccatcacccacttatcccttcatttgacaccattcattaagtgccttctgtgtgtcagtccctggccactcactgcagttcaaggccccctttccgctctgctgtactcctcgcctacctactccttgccttttctgtcgcacagccccttctttccaggcgagattcctcagcttctgagtaggaaacactccgggctccaggtttctggttgggaagggaaggccaggccaaaagctccaccggccgtatagataatgtactcgcagttttgtatcttccattcatactttaacctacaggtcatttgagtcttcacacaaataataacctatctggccaggagaattatctcagaacagaagtcatcagatcatcagagcccccagatggctacagaccagagattccacgctctcaggctgactagagtccgcatctcatctccaaactacacttccctggagaacaagtgccacaaaaatgaaaacaggccacttctcaggagttgaataatcaggggtcaccggaccccttggttgatgcactgcagcatggtggctttctgagtcctgttggccaccaagtgtcagcctcagcactcccgggactattgccaagaaggggcaagggatgagtcaagaaggtgagacccttcccggtgggcacgtgggccaggctgtgtgagatgttggatgtttggtactgtccatgtctgggtgtgtgcctattacctcagcatttctcacaaagtgtaccatgtagcatgttttgtgtatataaaagggagggtttttttaaaaatatattcccagattatccttgtaatgacacgaatctgcaataaaagccatcagtgct

SEQ ID NO: 5 - protein, 388 aa, Homo sapiens
MMSPSQASLLFLNVCIFICGEAVQGNCVHHSTDSSVVNIVEDGSNAKDESKSNDTVCKEDCEESCDVKTKITREEKHFMCRNLQNSIVSYTRSTKKLLRNMMDEQQASLDYLSNQVNELMNRVLLLTTEVFRKQLDPFPHRPVQSHGLDCTDIKDTIGSVTKTPSGLYIIHPEGSSYPFEVMCDMDYRGGGWTVIQKRIDGIIDFQRLWCDYLDGFGDLLGEFWLGLKKIFYIVNQKNTSFMLYVALESEDDTLAYASYDNFWLEDETRFFKMHLGRYSGNAGDAFRGLKKEDNQNAMPFSTSDVDNDGCRPACLVNGQSVKSCSHLHNKTGWWFNECGLANLNGIHHFSGKLLATGIQWGTWTKNNSPVKIKSVSMKIRRMYNPYFK

SEQ ID NO: 6 - DNA, 1820 bp, Homo sapiens
ctgactgatatttgaagaagtgttttcatctatccaagaaaaatatgatgtctccatcccaagcctcactcttattcttaaatgtatgtatttttatttgtggagaagctgtacaaggtaactgtgtacatcattctacggactcttcagtagttaacattgtagaagatggatctaatgcaaaagatgaaagtaaaagtaatgatactgtttgtaaggaagactgtgaggaatcatgtgatgttaaaactaaaattacacgagaagaaaaacatttcatgtgtagaaatttgcaaaattctattgtttcctacacaagaagtaccaaaaaactactaaggaatatgatggatgagcaacaagcttccttggattatttatctaatcaggttaacgagctcatgaatagagttctccttttgactacagaagtttttagaaaacagctggatccttttcctcacagacctgttcagtcacatggtttagattgcactgatattaaggataccattggctctgtcaccaaaacaccgagtggtttatacataattcacccagaaggatctagctacccatttgaggtaatgtgtgacatggattacagaggaggtggatggactgtgatacagaaaagaattgatgggataattgatttccagaggttgtggtgtgattatctggatggatttggagatcttctaggagaattttggctaggactgaaaaagattttttatatagtaaatcagaaaaataccagttttatgctgtatgtggctttggaatctgaagatgacactcttgcttatgcatcatatgataatttttggctagaggatgaaacgagattttttaaaatgcacttaggacggtattcaggaaatgctggtgatgcattccggggtctcaaaaaagaagataatcaaaatgcaatgccttttagcacatcagatgttgataatgatgggtgtcgccctgcatgcctggtcaatggtcagtctgtgaagagctgcagtcacctccataacaagaccggctggtggtttaacgagtgtggtctagcaaatctaaatggcattcatcacttctctggaaaattgcttgcaactggaattcaatggggcacgtggaccaaaaacaactcacctgtcaagattaaatctgtttcaatgaaaattagaagaatgtacaatccatattttaagtaatctcatttaacattgtaatgcaagttctacaatgataatatattaaagatttttaaaagtttatcttttcacttagtgtttcaaacatattaggcaaaatttaactgtagatggcatttagatgttatgagtttaattagaaaacttcaattttgtagtattctataaaagaaaacatggcttattgtatgtttttacttctgactatattaacaatatacaatgaaatttgtttcaagtgaactacaacttgtcttcctaaaatttatagtgattttaaaggattttgccttttctttgaagcatttttaaaccataatatgttgtaaggaaaattgaagggaatattttacttatttttatactttatatgattatataatctacagataatttctactgaagacagttacaataaataactttatgcagattaatatataagctacacatgatgtaaaaaccttactatttctaggtgatgccataccattttaaaagtagtaagagtttgctgcccaaatagtttttcttgttttcatatctaatcatggttaactattttgttattgtttgtaataaatatatgtacttttatatcctgaaaaa

SEQ ID NO: 7 - protein, 346 aa, Homo sapiens
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 8 - DNA, 2255 bp, Homo sapiens
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 9 - protein, 597 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 10 - DNA, 2937 bp, Homo sapiens
atttggttgaaagaaaacccacaatccagtgtcaagaaagaagtcaacttttcttcccctacttccctgcatttctcctctgtgctcactgccacacgcagctcaacctggacggcacagccagaggcgagatgcttctctgctgatctgagtctgcctgcagcatggacctgggtcttccctgaagcatctccagggctggagggacgactgccatgcaccgagggctcatccatccgcagagcagggcagtgggaggagacgccatgacccccatcgtcacagtcctgatctgtctcgggctgagtctgggccccaggacccacgtgcagacagggaccattcccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagaagaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcgtggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataatcaatgagttaaccgagaaaactaaaatcagaagtaagaatgtgctttaaactgaatcacaatataaatattacacatcacacaatgaaattgaaaaagtacaaaccacaaatgaaaaaagtagaaacgaaaaaaaaaaactaggaaatgaatgacgttggctttcgtataaggaatttagaaaaagaataaccaattattccaaatgaaggtgtaagaaagggaataagaagaagaagagttgctcatgaggaaaaaccaaaacttgaaaattcaacaaagccaatgaagctcattcttgaaaatattaattacagtcataaatcctaactacattgagcaagagaaagaaagagcaggcacgcatttccatatgggagtgagccagcagacagcccagcagatcctacacacattttcacaaactaaccccagaacaggctgcaaacctataccaatatactagaaaatgcagattaaatggatgaaatattcaaaactggagtttacataatgaacgtaagagtaatcagagaatctgactcattttaaatgtgtgtgtatgtgtgtgtatatatatgtgtgtgtgtgtgtgtgtgtgtgtgtgtgaaaaacattgactgtaataaaaatgttcccatcgtaaaaaaaaaaaaaaaa

SEQ ID NO: 11 - protein, 552 aa, Canis lupus familiaris
MGSSTSTLNLTVLLYLGLCWGPWDQAQAGTLPKPSIWADPGLMATKGSSVTLWCQTSLQADAYYLFKERVSRYFYMEISQDSKTKASYSIESMSAHEAGRYQCAYQIRKSWSQRSDLLTLVVTGVFGAPTLSANPGPVVASGVNMSLSCSSRYPWYSFHLLKEQGADVPQHLDLTILRERYRALFPVGPVNTSHGGTYRCYISEQSYPYSWSHPSDPLHLQVTGAYREPSLMAQPGSLVHSGENLTLQCRSEAGFDRFALTKDEELRPAQHLDGQPSPDFPLDPVSRTHGGRYRCYSGHNLSSTWSAPSAPLDILITGIYPKPSLSAQPGPSVSWGENVTLQCRSEIWFNTFHLSKEGSLAPPQHLHLQDTAIPYEVNFTLNPVTSDHQGTYRCYSSHNSSPYLLSSPSDSLELLVSASDQYLYILVGALVAFVLLLCLLVLFLVRQRHRGKGRKPGKKELKGPRDSGAPWADRGSSPAATTQEENLYAVIKDTQPEDSQQDSRVAISEDQQDVIYIQLNHLTLRQETSASSPSQSEEPPEEPSVYAALAFY

SEQ ID NO: 12 - DNA, 2162 bp, Canis lupus familiaris
ctcccaggccctgccctacctccccagcctggagacctggtacgatggccactctttggacatgggatctgtgaggaaaaggctctgaggactttataagctgtctgcacctctggggaccatgggaagcagcaccagcaccctgaacctcactgtccttctctacctcgggctgtgttggggaccatgggaccaggcacaggcagggaccctccccaagccctctatctgggctgacccaggcctcatggccaccaaggggagctctgtgaccctctggtgtcagacgtctctgcaggctgatgcttactatctgtttaaagagagggtctctaggtatttttacatggagatctcccaggattccaaaaccaaagccagttactccattgaatccatgagcgcacatgaggcagggcgataccagtgtgcatatcagatcaggaagagctggtcacagcggagtgacctcctgaccctggtggtgacaggagtatttggggcacccaccctctcagctaacccaggccctgtggtggcctcaggagtgaacatgtccctctcctgtagttcacgttacccatggtactcattccatctgctgaaggagcagggagctgatgtgccccaacacctggatttgacgatccttcgtgagaggtaccgggcactcttccctgtgggtcctgtgaacacctcccatgggggcacctacagatgctacatttctgaacagtcgtacccgtattcatggtcacaccccagtgaccccctgcaccttcaggtcacaggtgcctacagggagccttcactcatggcccagccgggctcactggttcattctggagaaaacctgaccctgcagtgtcgctcagaagctggctttgatagattcgctctgaccaaggatgaggagctcagacctgcccagcatctggatggtcaacccagccccgacttccccctggaccctgtgagccgcacccacgggggccggtacaggtgctacagtggacacaatctctcctccacgtggtcagcacccagtgcccctctggacatcctgatcacaggaatatacccaaaaccatccctctcagcccagcctgggccttcagtatcctggggagagaatgtgaccctgcagtgtcgctctgagatctggttcaataccttccacctgtccaaggaggggtcccttgctcctccccagcaccttcatctgcaggatacagctataccgtatgaggtcaacttcaccctgaatcctgtgacctcagaccaccaggggacctacaggtgctacagctcacacaactcctccccctacctgttgtcaagtcccagtgactccctggagctcttggtctcagcctcagaccagtacctgtacatccttgttggggccttagtggccttcgtcctgctgctctgcctcctcgtcctcttcctggtccgacagaggcatcgaggcaaaggcaggaagccgggtaagaaggagctgaagggccccagggatagtggtgccccatgggctgaccgaggctccagtcctgctgccaccacccaggaggagaacttgtatgccgtcataaaagacacgcagcctgaggacagccagcaggacagccgggtggctatatctgaggaccagcaagatgtgatctatatccaactgaaccatctgaccctccgacaggaaacaagtgcatcctctccctcccaatcagaggagcccccagaggagcccagtgtgtatgctgctctggccttctactagcccagaaggacccagaccccacactcgaggtcttaagactccctacagggaccccggaaggcacaggagctgctcacagcaggtaccacttgaccccagccagcctggaggcaggacaaggaccaccaggaacttcctggggtcttacgagagtcgattgcttccagaattcccatcagtacacagctcatcttagtttctgtaagatgccaaggaaatgctaacatcatttattaccacatggtaaccttgtggcgtgggggaacccagcactaagctggaaggactggggacctcctgcaggctccatggcagcctggcagcgtcccaggccatcactgacctcagagctcagcagtgccacagcccatgaggggttgct

SEQ ID NO: 13 - protein, 645 aa, Pan troglodytes
MTPILTVLICLGLSLGPRTHVQAGTLPKPTLRAEPDSVITQGSPVTLRCQGSLEAQENHLYREKKSASWIKRIQPQLVKKGQFPIPSITWEHAGRYRCQYYSRSQWSEPSDPPELVVTGAYSKPTLSALPSPVVTSGGNVTLQCGSQLAFGGFTLCKEGEDEHPQCVNSQSHTLGWSWAIFSVGPVSPSRRWSYRCYGYDWSSPYVWSLPSGLLELLVPGVSKKPSLSVQPGPVVAPGEILTLQCGSDVGYDRFVLYKEGERDFLQLPGRQPQAGLSQANFTLGPVSRSHGGQYRCCGAHNLSSEWSAPSDPLDILITGQIHARPSLSVQPGPTVASGENVTLLCQSQGWMHTFLLTKEGAADAPLRLGSTYRAQQYQAEFPMSPVTSAHAGTYRCYGSRSSNPYLLTHPSESLELVVSGPEDQPLTPTGSDPQSAPGLGSGAGTRAAGLGRHLGVVIGILVAVVLLLLLLLLLFLVLRHRRQGKRWTSTQRKADFQHPAGAVGPEPTDRGLQRRSSPAADAQEENLYAAVKDTQPEDGVEMDTQSPHDEDPQAVTYAPAKHSRPRREMASPPSPLSEEFLDTKDTQAEEDRQMDTEAAASEASQDVTYAQLHSLTLRREATEPPPSQEGPSPAEPSIYATLAIH

SEQ ID NO: 14 - DNA, 2213 bp, Pan troglodytes
ctgccatgccagcacccagggctcatccatgcacagagcaaggcagtgggaggagacgccatgacccccatcctcacggtcctgatctgtctcgggctgagtctgggccccaggacccacgtgcaggcagggaccctccccaagcccaccctcagggctgagccagactctgtgatcacccaggggagtcccgtgaccctcaggtgtcaggggagtcttgaagcccaggagaaccatctatatagggagaaaaaatcagcatcctggattaaacggatacaaccacagcttgtgaagaagggccagttccccatcccatccatcacctgggaacacgcagggcggtatcgctgtcagtattacagccgctctcagtggtcagagcccagtgatccccctgagctggtggtgacaggagcctacagcaaacctaccctctcagccctgcccagccctgtggtgacctcaggagggaacgtgaccctccagtgtggctcacagctggcatttggtggcttcactctgtgtaaggaaggagaagatgaacacccacaatgcgtgaactcccagtcccatacccttgggtggtcctgggccatcttctccgtgggccccgtgagcccgagtcgcaggtggtcgtacaggtgctatggttatgactggagctctccctatgtgtggtctctacccagtggtctcctggagctcctggtcccaggtgtttctaagaagccatcactctcagtgcagccaggtcctgtcgtggcccctggagagatcctgaccctccagtgtggctctgatgtcggctacgacagatttgttctgtataaggagggagaacgtgacttcctccagctccctggccggcagccccaggctgggctctcccaggccaacttcaccctgggccctgtgagccgctcccacgggggccagtacagatgctgcggtgcacacaacctctcctccgagtggtcggcccccagtgaccccctggacatcctgatcacaggacagatccatgccagaccctccctctcggtgcagccgggccccacagtggcctcaggagagaacgtgaccctgctgtgtcagtcacagggatggatgcacactttccttctgaccaaggagggagcagctgatgccccgctgcgtctaggctccacttacagagctcaacagtaccaggctgaatttcccatgagtcctgtgacctcagcccacgcggggacctacaggtgctacggctcacgcagctccaacccctacctgctgactcaccccagtgagtccctggagctcgtggtctcaggccctgaggaccagcccctcacccccaccgggtcggatccccagagtgctccgggactcggctctggtgcaggaacaagggctgcaggtctgggaaggcacctgggggttgtgatcggcatcttggtggccgtcgtcctcctgctcctcctcctcctcctcctgttcctcgtcctccgacaccgacgtcagggcaaacgctggacatcgacccagagaaaggctgatttccagcatcctgcaggggctgtggggccagagcccacagacagaggcctgcagaggaggtccagcccagctgccgatgcccaggaagaaaacctctatgctgccgtgaaggacacacagcctgaggacggggtggagatggacactcagagcccacacgatgaagacccccaggcagtgacgtatgccccggcgaaacactccagacctaggagagaaatggcctctcctccctccccactgtccgaggaattcctggacacaaaggacacacaggcggaagaggacaggcagatggacactgaggctgctgcatctgaagcctcccaggatgtgacctacgcccagctgcacagcttgaccctcagacgggaggcaactgagcctcctccatcccaggaagggccctctccagctgagcccagcatctacgccaccctggccatccactagcccggagggtaggcagactccacactcagtagaaggagactcaggactgctgaaggcacgggagctgcccccagtggacaccagtgaaccccagccagcctggacccctaacaaagaccatgaggagatgctgggaactttgggactcacttgcttctgcggtcgaaataactaatatccctacattttttaattaaagcaacagacttctcaat

SEQ ID NO: 15 - protein, 11 aa, Artificial
  HA peptide
SFERFEIFPKE

SEQ ID NO: 16 - DNA, 21 bp, Artificial
  GAPDH Primer
tggagattgttgccatcaacg

SEQ ID NO: 17 - DNA, 25 bp, Artificial
  GAPDH Primer
cagtggatgcagggatgatgttctg

SEQ ID NO: 18 - DNA, 22 bp, Artificial
  Foxp3 Primer
cagctgcctacagtgcccctag

SEQ ID NO: 19 - DNA, 20 bp, Artificial
  Foxp3 Primer
catttgccagcagtgggtag

SEQ ID NO: 20 - DNA, 97 bp, Artificial
  LILRB2 shRNA
tgctgttgacagtgagcgccagcttgaccctcagacggaatagtgaagccacagatgtattccgtctgagggtcaagctgttgcctactgcctcgga

SEQ ID NO: 21 - DNA, 97 bp, Artificial
  LILRB2 shRNA
tgctgttgacagtgagcgcacgaccagagcttgtgaagaatagtgaagccacagatgtattcttcacaagctctggtcgtatgcctactgcctcgga

SEQ ID NO: 22 - DNA, 97 bp, Artificial
  Angptl-1 shRNA
tgctgttgacagtgagcgcctcgtgttactcaactctatatagtgaagccacagatgtatatagagttgagtaacacgagatgcctactgcctcgga

SEQ ID NO: 23 - DNA, 97 bp, Artificial
  Angptl-1 shRNA
tgctgttgacagtgagcgaagagacactcgccaatttaaatagtgaagccacagatgtatttaaattggcgagtgtctctctgcctactgcctcgga

SEQ ID NO: 24 - DNA, 97 bp, Artificial
  Angptl-1 shRNA
tgctgttgacagtgagcgaccaatttaaatgacacagaactagtgaagccacagatgtagttctgtgtcatttaaattggctgcctactgcctcgga

SEQ ID NO: 25 - DNA, 97 bp, Artificial
  Angptl-2 shRNA
tgctgttgacagtgagcgccacagagttcttggaataaaatagtgaagccacagatgtattttattccaagaactctgtgatgcctactgcctcgga

SEQ ID NO: 26 - DNA, 97 bp, Artificial
  Angptl-2 shRNA
tgctgttgacagtgagcgacacagcagcggcagaagcttatagtgaagccacagatgtataagcttctgccgctgctgtggtgcctactgcctcgga

SEQ ID NO: 27 - DNA, 97 bp, Artificial
  Angptl-2 shRNA
tgctgttgacagtgagcgccagatggaggctggacagtaatagtgaagccacagatgtattactgtccagcctccatctgatgcctactgcctcgga

SEQ ID NO: 28 - DNA, 97 bp, Artificial
  Angptl-3 shRNA
tgctgttgacagtgagcgactcagaaggactagtattcaatagtgaagccacagatgtattgaatactagtccttctgagctgcctactgcctcgga

SEQ ID NO: 29 - DNA, 97 bp, Artificial
  Angptl-3 shRNA
tgctgttgacagtgagcgccagcatagtcaaataaaagaatagtgaagccacagatgtattcttttatttgactatgctgttgcctactgcctcgga

SEQ ID NO: 30 - DNA, 97 bp, Artificial
  Angptl-3 shRNA
tgctgttgacagtgagcgatacatataaactacaagtcaatagtgaagccacagatgtattgacttgtagtttatatgtagtgcctactgcctcgga

SEQ ID NO: 31 - DNA, 97 bp, Artificial; identical to SEQ ID NO: 25
  Angptl-4 shRNA

SEQ ID NO: 32 - DNA, 97 bp, Artificial; identical to SEQ ID NO: 26
  Angptl-4 shRNA

SEQ ID NO: 33 - DNA, 97 bp, Artificial; identical to SEQ ID NO: 27
  Angptl-4 shRNA

SEQ ID NO: 34 - DNA, 97 bp, Artificial
  Angptl-5 shRNA
tgctgttgacagtgagcgatagaagatggatctaatgcaatagtgaagccacagatgtattgcattagatccatcttctactgcctactgcctcgga

SEQ ID NO: 35 - DNA, 97 bp, Artificial
  Angptl-5 shRNA
tgctgttgacagtgagcgaatggtttagattgcactgatatagtgaagccacagatgtatatcagtgcaatctaaaccatgtgcctactgcctcgga

SEQ ID NO: 36 - DNA, 97 bp, Artificial
  Angptl-5 shRNA
tgctgttgacagtgagcgatacggactcttcagtagttaatagtgaagccacagatgtattaactactgaagagtccgtagtgcctactgcctcgga

SEQ ID NO: 37 - DNA, 97 bp, Artificial
  Angptl-6 shRNA
tgctgttgacagtgagcgccactacctggcagcactataatagtgaagccacagatgtattatagtgctgccaggtagtgatgcctactgcctcgga

SEQ ID NO: 38 - DNA, 97 bp, Artificial
  Angptl-6 shRNA
tgctgttgacagtgagcgagaggcaagatggttcagtcaatagtgaagccacagatgtattgactgaaccatcttgcctcctgcctactgcctcgga

SEQ ID NO: 39 - DNA, 97 bp, Artificial
  Angptl-6 shRNA
tgctgttgacagtgagcgacccagagagaccagacccagatagtgaagccacagatgtatctgggtctggtctctctggggtgcctactgcctcgga

SEQ ID NO: 40 - DNA, 97 bp, Artificial
  Angptl-7 shRNA
tgctgttgacagtgagcgcccgggactggaagcagtacaatagtgaagccacagatgtattgtactgcttccagtcccggttgcctactgcctcgga

SEQ ID NO: 41 - DNA, 97 bp, Artificial
  Angptl-7 shRNA
tgctgttgacagtgagcgcccgcatctctggagtgtataatagtgaagccacagatgtattatacactccagagatgcggttgcctactgcctcgga

SEQ ID NO: 42 - DNA, 97 bp, Artificial
  Angptl-7 shRNA
tgctgttgacagtgagcgcggactgagaaacagcctataatagtgaagccacagatgtattataggctgtttctcagtccttgcctactgcctcgga

SEQ ID NO: 43 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 44 - DNA, 2126 bp, Homo sapiens
ttccagaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatt

SEQ ID NO: 45 - protein, 503 aa, Homo sapiens
MLSQLAMLQGSLLLVVATMSVAQQTRQEADRGCETLVVQHGHCSYTFLLPKSEPCPPGPEVSRDSNTLQRESLANPLHLGKLPTQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLSRQSAALTNIERGLRGVRHNSSLLQDQQHSLRQLLVLLRHLVQERANASAPAFIMAGEQVFQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 46 - DNA, 1955 bp, Homo sapiens
tgcagctgcaggcaagcctggccactgttggctgcagcaggacatcccaggcacagcccctagggctctgagcagacatccctcgccattgacacatcttcagatgctctcccagctagccatgctgcagggcagcctcctccttgtggttgccaccatgtctgtggctcaacagacaaggcaggaggcggataggggctgcgagacacttgtagtccagcacggccactgtagctacaccttcttgctgcccaagtctgagccctgccctccggggcctgaggtctccagggactccaacaccctccagagagaatcactggccaacccactgcacctggggaagttgcccacccagcaggtgaaacagctggagcaggcactgcagaacaacacgcagtggctgaagaagctagagagggccatcaagacgatcttgaggtcgaagctggagcaggtccagcagcaaatggcccagaatcagacggcccccatgctagagctgggcaccagcctcctgaaccagaccactgcccagatccgcaagctgaccgacatggaggctcagctcctgaaccagacatcaagaatggatgcccagatgccagagacctttctgtccaccaacaagctggagaaccagctgctgctacagaggcagaagctccagcagcttcagggccaaaacagcgcgctcgagaagcggttgcaggccctggagaccaagcagcaggaggagctggccagcatcctcagcaagaaggcgaagctgctgaacacgctgagccgccagagcgccgccctcaccaacatcgagcgcggcctgcgcggtgtcaggcacaactccagcctcctgcaggaccagcagcacagcctgcgccagctgctggtgttgttgcggcacctggtgcaagaaagggctaacgcctcggccccggccttcataatggcaggtgagcaggtgttccaggactgtgcagagatccagcgctctggggccagtgccagtggtgtctacaccatccaggtgtccaatgcaacgaagcccaggaaggtgttctgtgacctgcagagcagtggaggcaggtggaccctcatccagcgccgtgagaatggcaccgtgaattttcagcggaactggaaggattacaaacagggcttcggagacccagctggggagcactggctgggcaatgaagtggtgcaccagctcaccagaagggcagcctactctctgcgtgtggagctgcaagactgggaaggccacgaggcctatgcccagtacgaacatttccacctgggcagtgagaaccagctatacaggctttctgtggtcgggtacagcggctcagcagggcgccagagcagcctggtcctgcagaacaccagctttagcacccttgactcagacaacgaccactgtctctgcaagtgtgcccaagtgatgtctggagggtggtggtttgacgcctgtggcctgtcaaacctcaacggcgtctactaccacgctcccgacaacaagtacaagatggacggcatccgctggcactacttcaagggccccagctactcactgcgtgcctctcgcatgatgatacggcctttggacatctaacgagcagctgtgccagaggctggaccacacaggagaagctcggacttggcactcctggacaacctggacccagatgcaagacactgtgccaccgccttccctgacaccctgggcttcctgagccagccctccttgacccagaagtccagaagggtcatctgccccccaactcccctccgtctgtgacatggagggtgttcggggcccatccctctgatgtagtcctcgcccctcttctctccctcccccttcaggggctccctgcctgagggtcacagtaccttgaatgggctgagaacagaccaaaaaaaaaaaaaaaaaaaaaaaaaaaaaaaaaaaa

SEQ ID NO: 47 - protein, 470 aa, Homo sapiens
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASPMPAGHPAVPTKPVGPWQDCAEARQAGHEQSGVYELRVGRHVVSVWCEQQLEGGGWTVIQRRQDGSVNFFTTWQHYKAGFGRPDGEYWLGLEPVYQLTSRGDHELLVLLEDWGGRGARAHYDGFSLEPESDHYRLRLGQYHGDAGDSLSWHNDKPFSTVDRDRDSYSGNCALYQRGGWWYHACAHSNLNGVWHHGGHYRSRYQDGVYWAEFRGGAYSLRKAAMLIRPLKL

SEQ ID NO: 48 - DNA, 1857 bp, Homo sapiens
gcatcccagctccactcccaggctctgggggctggggagtggttaccaagcctcctctctccttctgtcccactgccctctccccgtctctagctcagaggccccactggaccctcggctcttccttggacttcttgtgtgttctgtgagcttcgctggattcagggtcttgggcatcagaggtccgccgcgatggggaagccctggctgcgtgcgctacagctgctgctcctgctgggcgcgtcgtgggcgcgggcgggcgccccgcgctgcacctacaccttcgtgctgcccccgcagaagttcacgggcgctgtgtgctggagcggccccgcatccacgcgggcgacgcccgaggccgccaacgccagcgagctggcggcgctgcgcatgcgcgtcggccgccacgaggagctgttacgcgagctgcagaggctggcggcggccgacggcgccgtggccggcgaggtgcgcgcgctgcgcaaggagagccgcggcctgagcgcgcgcctgggccagttgcgcgcgcagctgcagcacgaggcggggcccggggcgggcccgggggcggatctgggggcggagcctgccgcggcgctggcgctgctcggggagcgcgtgctcaacgcgtccgccgaggctcagcgcgcagccgcccggttccaccagctggacgtcaagttccgcgagctggcgcagctcgtcacccagcagagcagtctcatcgcccgcctggagcgcctgtgcccgggaggcgcgggcgggcagcagcaggtcctgccgccacccccactggtgcctgtggttccggtccgtcttgtgggtagcaccagtgacaccagtaggatgctggacccagccccagagccccagagagaccagacccagagacagcaggagcccatggcttctcccatgcctgcaggtcaccctgcggtccccaccaagcctgtgggcccgtggcaggattgtgcagaggcccgccaggcaggccatgaacagagtggagtgtatgaactgcgagtgggccgtcacgtagtgtcagtatggtgtgagcagcaactggagggtggaggctggactgtgatccagcggaggcaagatggttcagtcaacttcttcactacctggcagcactataaggcgggctttgggcggccagacggagaatactggctgggccttgaacccgtgtatcagctgaccagccgtggggaccatgagctgctggttctcctggaggactgggggggccgtggagcacgtgcccactatgatggcttctccctggaacccgagagcgaccactaccgcctgcggcttggccagtaccatggtgatgctggagactctctttcctggcacaatgacaagcccttcagcaccgtggatagggaccgagactcctattctggtaactgtgccctgtaccagcggggaggctggtggtaccatgcctgtgcccactccaacctcaacggtgtgtggcaccacggcggccactaccgaagccgctaccaggatggtgtctactgggctgagtttcgtggtggggcatattctctcaggaaggccgccatgctcattcggcccctgaagctgtgactctgtgttcctctgtcccctaggccctagaggacattggtcagcaggagcccaagttgttctggccacaccttctttgtggctcagtgccaatgtgtcccacagaacttcccactgtggatctgtgaccctgggcgctgaaaatgggacccaggaatcccccccgtcaatatcttggcctcagatggctccccaaggtcattcatatctcggtttgagctcatatcttataataacacaaagtagccacag

SEQ ID NO: 49 - protein, 1857 aa, Homo sapiens
GCATCCCAGCTCCACTCCCAGGCTCTGGGGGCTGGGGAGTGGTTACCAAGCCTCCTCTCTCCTTCTGTCCCACTGCCCTCTCCCCGTCTCTAGCTCAGAGGCCCCACTGGACCCTCGGCTCTTCCTTGGACTTCTTGTGTGTTCTGTGAGCTTCGCTGGATTCAGGGTCTTGGGCATCAGAGGTCCGCCGCGATGGGGAAGCCCTGGCTGCGTGCGCTACAGCTGCTGCTCCTGCTGGGCGCGTCGTGGGCGCGGGCGGGCGCCCCGCGCTGCACCTACACCTTCGTGCTGCCCCCGCAGAAGTTCACGGGCGCTGTGTGCTGGAGCGGCCCCGCATCCACGCGGGCGACGCCCGAGGCCGCCAACGCCAGCGAGCTGGCGGCGCTGCGCATGCGCGTCGGCCGCCACGAGGAGCTGTTACGCGAGCTGCAGAGGCTGGCGGCGGCCGACGGCGCCGTGGCCGGCGAGGTGCGCGCGCTGCGCAAGGAGAGCCGCGGCCTGAGCGCGCGCCTGGGCCAGTTGCGCGCGCAGCTGCAGCACGAGGCGGGGCCCGGGGCGGGCCCGGGGGCGGATCTGGGGGCGGAGCCTGCCGCGGCGCTGGCGCTGCTCGGGGAGCGCGTGCTCAACGCGTCCGCCGAGGCTCAGCGCGCAGCCGCCCGGTTCCACCAGCTGGACGTCAAGTTCCGCGAGCTGGCGCAGCTCGTCACCCAGCAGAGCAGTCTCATCGCCCGCCTGGAGCGCCTGTGCCCGGGAGGCGCGGGCGGGCAGCAGCAGGTCCTGCCGCCACCCCCACTGGTGCCTGTGGTTCCGGTCCGTCTTGTGGGTAGCACCAGTGACACCAGTAGGATGCTGGACCCAGCCCCAGAGCCCCAGAGAGACCAGACCCAGAGACAGCAGGAGCCCATGGCTTCTCCCATGCCTGCAGGTCACCCTGCGGTCCCCACCAAGCCTGTGGGCCCGTGGCAGGATTGTGCAGAGGCCCGCCAGGCAGGCCATGAACAGAGTGGAGTGTATGAACTGCGAGTGGGCCGTCACGTAGTGTCAGTATGGTGTGAGCAGCAACTGGAGGGTGGAGGCTGGACTGTGATCCAGCGGAGGCAAGATGGTTCAGTCAACTTCTTCACTACCTGGCAGCACTATAAGGCGGGCTTTGGGCGGCCAGACGGAGAATACTGGCTGGGCCTTGAACCCGTGTATCAGCTGACCAGCCGTGGGGACCATGAGCTGCTGGTTCTCCTGGAGGACTGGGGGGGCCGTGGAGCACGTGCCCACTATGATGGCTTCTCCCTGGAACCCGAGAGCGACCACTACCGCCTGCGGCTTGGCCAGTACCATGGTGATGCTGGAGACTCTCTTTCCTGGCACAATGACAAGCCCTTCAGCACCGTGGATAGGGACCGAGACTCCTATTCTGGTAACTGTGCCCTGTACCAGCGGGGAGGCTGGTGGTACCATGCCTGTGCCCACTCCAACCTCAACGGTGTGTGGCACCACGGCGGCCACTACCGAAGCCGCTACCAGGATGGTGTCTACTGGGCTGAGTTTCGTGGTGGGGCATATTCTCTCAGGAAGGCCGCCATGCTCATTCGGCCCCTGAAGCTGTGACTCTGTGTTCCTCTGTCCCCTAGGCCCTAGAGGACATTGGTCAGCAGGAGCCCAAGTTGTTCTGGCCACACCTTCTTTGTGGCTCAGTGCCAATGTGTCCCACAGAACTTCCCACTGTGGATCTGTGACCCTGGGCGCTGAAAATGGGACCCAGGAATCCCCCCCGTCAATATCTTGGCCTCAGATGGCTCCCCAAGGTCATTCATATCTCGGTTTGAGCTCATATCTTATAATAACACAAAGTAGCCACAG

SEQ ID NO: 50 - DNA, 2094 bp, Homo sapiens
cacttgttcaatgatgtacccccagtgtcaggcgctttgcaaacacacgatacatacgggttgatgtttggtcaagagaggaattaagaccaggcagacagcaggctgggatcagagagaccccatttctgtctgaaatgtctgcagagaacctggtgcctgcctcagccctagctctggggaaatgaaagccaggctggggttcaaatgagggcagtttcccttcctgtgggctgctgatggaacaaccccatgacgagaaggacccagcctccaagcggccacaccctgtgtgtctctttgtcctgccggcactgaggactcatccatctgcacagctggggcccctgggaggagacgccatgatccccaccttcacggctctgctctgcctcgggctgagtctgggccccaggacccacatgcaggcagggcccctccccaaacccaccctctgggctgagccaggctctgtgatcagctgggggaactctgtgaccatctggtgtcaggggaccctggaggctcgggagtaccgtctggataaagaggaaagcccagcaccctgggacagacagaacccactggagcccaagaacaaggccagattctccatcccatccatgacagaggactatgcagggagataccgctgttactatcgcagccctgtaggctggtcacagcccagtgaccccctggagctggtgatgacaggagcctacagtaaacccaccctttcagccctgccgagtcctcttgtgacctcaggaaagagcgtgaccctgctgtgtcagtcacggagcccaatggacactttccttctgatcaaggagcgggcagcccatcccctactgcatctgagatcagagcacggagctcagcagcaccaggctgaattccccatgagtcctgtgacctcagtgcacggggggacctacaggtgcttcagctcacacggcttctcccactacctgctgtcacaccccagtgaccccctggagctcatagtctcaggatccttggaggatcccaggccctcacccacaaggtccgtctcaacagctgcaggccctgaggaccagcccctcatgcctacagggtcagtcccccacagtggtctgagaaggcactgggaggtactgatcggggtcttggtggtctccatcctgcttctctccctcctcctcttcctcctcctccaacactggcgtcagggaaaacacaggacattggcccagagacaggctgatttccaacgtcctccaggggctgccgagccagagcccaaggacgggggcctacagaggaggtccagcccagctgctgacgtccagggagaaaacttctgtgctgccgtgaagaacacacagcctgaggacggggtggaaatggacactcggagcccacacgatgaagacccccaggcagtgacgtatgccaaggtgaaacactccagacctaggagagaaatggcctctcctccctccccactgtctggggaattcctggacacaaaggacagacaggcagaagaggacagacagatggacactgaggctgctgcatctgaagccccccaggatgtgacctacgcccagctgcacagctttaccctcagacagaaggcaactgagcctcctccatcccaggaaggggcctctccagctgagcccagtgtctatgccactctggccatccactaatccaggggggacccagaccccacaagccatggagactcaggaccccagaaggcatggaagctgcctccagtagacatcactgaaccccagccagcccagacccctgacacagaccactagaagattccgggaacgttgggagtcacctgattctgcaaagataaataatatccctgcattatcaaaataaagtagcagacctctcaattcacaatgagttaactgataaaacaaaacagaagtcagacaatgttttaaattgaatgatcatgtaaatattacacatcaaaccaatgacatgggaaaatgggagcttctaatgaggacaaacaaaaaatagagaaaaattaataaagtcaaaatgtttattcttgaaaaaaaaaaaaaa

SEQ ID NO: 51 - protein, 335 aa, Mus musculus
MIAMLTVLLYLGLILEPRTAVQAGHLPKPIIWAEPGSVIAAYTSVITWCQGSWEAQYYHLYKEKSVNPWDTQVPLETRNKAKFNIPSMTTSYAGIYKCYYESAAGFSEHSDAMELVMTGAYENPSLSVYPSSNVTSGVSISFSCSSSIVFGRFILIQEGKHGLSWTLDSQHQANQPSYATFVLDAVTPNHNGTFRCYGYFRNEPQVWSKPSNSLDLMISETKDQSSTPTEDGLETYQKILIGVLVSFLLLFFLLLFLILIGYQYGHKKKANASVKNTQSENNAELNSWNPQNEDPQGIVYAQVKPSRLQKDTACKETQDVTYAQLCIRTQEQNNS

SEQ ID NO: 52 - DNA, 1420 bp, Mus musculus
gactggcccaagagaacagctacgaacattgcctggactcaccatgatcgccatgctcacagtgctgctataccttggtcttattctggaacccaggactgcagtacaggcaggacacctcccaaagcccatcatctgggctgagccaggctctgtgatcgctgcgtatacatctgtgattacctggtgtcagggttcctgggaggcccagtattatcatctgtataaagagaaaagtgtaaatccttgggacactcaagtccctctggaaaccaggaataaggccaagttcaacattccaagcatgacaacctcatatgcaggcatatataagtgttactatgagagtgctgctggcttctcagagcacagtgatgccatggagctggtgatgacaggagcatatgaaaatcccagcctgtcagtctatcccagctctaatgtgacctctggagtttccatatcctttagttgcagctcatccatagtatttggcagattcattctgatccaggaaggaaagcatggcctctcttggaccctggactcacagcatcaggccaatcagccatcctatgctacttttgttctggatgctgttactcccaaccacaatggaacattcagatgctatggctactttagaaatgaaccacaggtgtggtcaaaaccaagtaactccctagacctcatgatctcagaaaccaaggaccagtcctctacacccactgaagatggactggaaacataccagaagattttgattggagtcctggtgtcattcctcctgcttttcttcctcctgctttttctcatcctcatcggataccagtatgggcacaaaaagaaggctaatgcttctgtgaagaacacacaatctgagaacaatgcagagctgaacagttggaacccacaaaatgaagacccccagggaattgtctacgcccaggtaaaaccctccaggcttcagaaggacactgcatgcaaagagacccaggatgtaacctatgcccagttgtgcatcaggacacaggaacagaacaacagctgaaaccagcaagcagccaacacagaaagaagacccagaccccacattccatgaatgaaaactagggattttctggtatttgatcccagtggactctatttaattaattcaggccatcatgaatacctcatgtgttatcagaagcttctcagaattttgggaatatactttattctttgttcagagataacaaatgtcattacattttgtaaataaagccaaatcctcctcaataaacaattcatctaatgagaaacaaatgaaatgagatattgtgaattgaaagacaatgtaaccagggcacactaatcaatgaaacttgaatttttagaaccatgaataaatgtataagcaaagaaagtgtcttcaaaa

SEQ ID NO: 53 - protein, 337 aa, Rat rattus
MISMLTMLLYLGLILEHRTKIQAGHLPRPIIWAEPGSMIAIYTSVTIWCQGSWEAQKYHLYKEGNVNPWDTQLPLETRNKAKFNIQYMTTTYADTYKCYYESAAGFSEHSDAMELVMTGAYTNPSLSVWPSSDVTSGVSIAFKCSSSMGFGRFILIQDGKDSFRWTLDSQRHDNQPFHATFVLDTVTPNHNGTFRCYGSFRNEPHLWSKSSDPLVLMVSETKNQSPTHTEDAQRDANLQLTSGVPETTYRDRLHQKSSSPDATINEKYLYASVKNTQSEDNGELDSWNPPDKDPHGIVYAQVKPSRLRKDTTSWRPKETQVVTYAQLCSRTQEYDNR

SEQ ID NO: 54 - DNA, 1152 bp, Rat rattus
ggagtcatcatgatctccatgctcaccatgctgctgtaccttggtctcattttggaacacaggactaaaatacaggcaggacaccttccaaggcccatcatctgggccgagccaggctctatgattgccatttatacatctgtgactatctggtgtcaggggtcttgggaggcccagaagtatcatctgtataaagagggaaatgtaaatccttgggacactcaactccctctggaaaccaggaataaggctaagttcaacattcaatacatgacaaccacttatgcagacacatataagtgttactatgagagtgctgctggtttttcagagcacagtgatgccatggagctggtgatgacaggagcctatacaaatcccagcctgtcagtctggcccagttctgatgtgacctctggagtttccatagcctttaagtgtagctcatccatgggatttggcagattcattctgatccaggacggaaaagacagctttcgatggaccctggactcacaacgacatgacaaccaaccattccatgctacttttgttctggacactgttactcccaaccacaatggaacattcagatgctatggctcttttagaaatgaaccacatttgtggtcgaaatcaagtgatccccttgtcctcatggtatcagaaaccaagaaccagtctcctacacacactgaagatgctcaaagagatgccaacttacaattgacttcaggggttccagaaacaacatacagagatagactccaccagaagagctccagcccagatgctaccatcaatgaaaaatacctgtatgcttctgtgaaaaacacacaatctgaggacaatggagagctggacagttggaacccacctgataaagacccccatggaattgtgtacgcccaggtgaaaccctccagacttcggaaggatactacatcctggagacccaaagagacccaggttgtaacctacgcccagttgtgcagcaggactcaggaatatgacaaccgctgagaccagcaagcagccaaagcaagaagaagacccagacccaacattccatgaatgaaaactagaaattttctaagacagtatttgtttccagtggactctgtttaattaatggcagccatcatgaatcat

SEQ ID NO: 55 - protein, 1152 aa, Homo sapiens
GGAGTCATCATGATCTCCATGCTCACCATGCTGCTGTACCTTGGTCTCATTTTGGAACACAGGACTAAAATACAGGCAGGACACCTTCCAAGGCCCATCATCTGGGCCGAGCCAGGCTCTATGATTGCCATTTATACATCTGTGACTATCTGGTGTCAGGGGTCTTGGGAGGCCCAGAAGTATCATCTGTATAAAGAGGGAAATGTAAATCCTTGGGACACTCAACTCCCTCTGGAAACCAGGAATAAGGCTAAGTTCAACATTCAATACATGACAACCACTTATGCAGACACATATAAGTGTTACTATGAGAGTGCTGCTGGTTTTTCAGAGCACAGTGATGCCATGGAGCTGGTGATGACAGGAGCCTATACAAATCCCAGCCTGTCAGTCTGGCCCAGTTCTGATGTGACCTCTGGAGTTTCCATAGCCTTTAAGTGTAGCTCATCCATGGGATTTGGCAGATTCATTCTGATCCAGGACGGAAAAGACAGCTTTCGATGGACCCTGGACTCACAACGACATGACAACCAACCATTCCATGCTACTTTTGTTCTGGACACTGTTACTCCCAACCACAATGGAACATTCAGATGCTATGGCTCTTTTAGAAATGAACCACATTTGTGGTCGAAATCAAGTGATCCCCTTGTCCTCATGGTATCAGAAACCAAGAACCAGTCTCCTACACACACTGAAGATGCTCAAAGAGATGCCAACTTACAATTGACTTCAGGGGTTCCAGAAACAACATACAGAGATAGACTCCACCAGAAGAGCTCCAGCCCAGATGCTACCATCAATGAAAAATACCTGTATGCTTCTGTGAAAAACACACAATCTGAGGACAATGGAGAGCTGGACAGTTGGAACCCACCTGATAAAGACCCCCATGGAATTGTGTACGCCCAGGTGAAACCCTCCAGACTTCGGAAGGATACTACATCCTGGAGACCCAAAGAGACCCAGGTTGTAACCTACGCCCAGTTGTGCAGCAGGACTCAGGAATATGACAACCGCTGAGACCAGCAAGCAGCCAAAGCAAGAAGAAGACCCAGACCCAACATTCCATGAATGAAAACTAGAAATTTTCTAAGACAGTATTTGTTTCCAGTGGACTCTGTTTAATTAATGGCAGCCATCATGAATCAT

SEQ ID NO: 56 - DNA, 2859 bp, Homo sapiens
aagtcaacttttcttccctatttccctgcatttctcttctgtgctcgctgccacacgcagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggccccaggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcactctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaatcctccagtgtgactcacaggtggcatttgatggcttcagtctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaa

SEQ ID NO: 57 - protein, 632 aa, Homo sapiens
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 58 - DNA, 2843 bp, Homo sapiens
atgacaagaaggacccagcctccgagcggccacaccctgtgtgtctctctgtcctgccagcactgagggctcatccctctgcagagcgcggggtcaccgggaggagacgccatgacgcccgccctcacagccctgctctgccttgggctgagtctgggccccaggacccgcgtgcaggcagggcccttccccaaacccaccctctgggctgagccaggctctgtgatcagctgggggagccccgtgaccatctggtgtcaggggagccaggaggcccaggagtaccgactgcataaagagggaagcccagagcccttggacagaaataacccactggaacccaagaacaaggccagattctccatcccatccatgacagagcaccatgcagggagataccgctgccactattacagctctgcaggctggtcagagcccagcgaccccctggagatggtgatgacaggagcctacagcaaacccaccctctcagccctgcccagccctgtggtggcctcaggggggaatatgaccctccgatgtggctcacagaagggatatcaccattttgttctgatgaaggaaggagaacaccagctcccccggaccctggactcacagcagctccacagtcgggggttccaggccctgttccctgtgggccccgtgacccccagccacaggtggaggttcacatgctattactattatacaaacaccccctgggtgtggtcccaccccagtgaccccctggagattctgccctcaggcgtgtctaggaagccctccctcctgaccctgcagggccctgtcctggcccctgggcagagcctgaccctccagtgtggctctgatgtcggctacaacagatttgttctgtataaggagggggaacgtgacttcctccagcgccctggccagcagccccaggctgggctctcccaggccaacttcaccctgggccctgtgagcccctccaatgggggccagtacaggtgctacggtgcacacaacctctcctccgagtggtcggcccccagcgaccccctgaacatcctgatggcaggacagatctatgacaccgtctccctgtcagcacagccgggccccacagtggcctcaggagagaacgtgaccctgctgtgtcagtcatggtggcagtttgacactttccttctgaccaaagaaggggcagcccatcccccactgcgtctgagatcaatgtacggagctcataagtaccaggctgaattccccatgagtcctgtgacctcagcccacgcggggacctacaggtgctacggctcatacagctccaacccccacctgctgtctcaccccagtgagcccctggagctcgtggtctcaggacactctggaggctccagcctcccacccacagggccgccctccacacctggtctgggaagatacctggaggttttgattggggtctcggtggccttcgtcctgctgctcttcctcctcctcttcctcctcctccgacgtcagcgtcacagcaaacacaggacatctgaccagagaaagactgatttccagcgtcctgcaggggctgcggagacagagcccaaggacaggggcctgctgaggaggtccagcccagctgctgacgtccaggaagaaaacctctatgctgccgtgaaggacacacagtctgaggacagggtggagctggacagtcagcagagcccacacgatgaagacccccaggcagtgacgtatgccccggtgaaacactccagtcctaggagagaaatggcctctcctccctcctcactgtctggggaattcctggacacaaaggacagacaggtggaagaggacaggcagatggacactgaggctgctgcatctgaagcctcccaggatgtgacctacgcccagctgcacagcttgacccttagacggaaggcaactgagcctcctccatcccaggaaggggaacctccagctgagcccagcatctacgccactctggccatccactagcccggggggtacgcagaccccacactcagcagaaggagactcaggactgctgaaggcacgggagctgcccccagtggacaccagtgaaccccagtcagcctggacccctaacacagaccatgaggagacgctgggaacttgtgggactcacctgactcaaagatgactaatatcgtcccattttggaaataaagcaacagacttctcaacaatcaatgagttaataacaaaaaaacaaaaaacaaaaacagacgtaaaggccgggtgtggtactcaggaggctgagtggggaggattccttgaacacaagaagttaaggctgctgaggctgcagtgagctatgactgtgccactgcactccagcctgtgtgacagagcgagaccttgtctctaaaaaaaaaaacagtgaatgttttaaactgaatgataatgtaaatattatacatcgaacttatgacatgggaaaattaagaagcataaataggccgggcgcggtggctcacgcctataatctcagcactttgggaggctgatgcgggcggatcatgaggtcaggagatcgagaccatcctggctaacacggtgaaaccccgtctctactaaaaatacaaaaaaattagccgggcgtggtggcgagtgcctatagtcccagctactcaggaggctgaggcaggagaatggcatgagcccgggaggcagagcttgcagtgagctgagatcgcaccactgcactccagcctgggcgacagagtgagattccgtctcgaaaaaaaaaaaaaaagaaagaaaaaaaataaaaaagaagcataaccaggaaaaaaaaaaaaaaaaaa

SEQ ID NO: 59 - protein, 591 aa, Homo sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPAGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 60 - DNA, 2282 bp, Homo sapiens
cttgtgacagggagaacccagcctccagtccacactctgcgtgtttttgtgtcctgccaggcaccgtggtctcatccgcctgcacagctgagtccagtgggagctgacgccatgaccctcaccctctcagtcctgatttgcctcgggctgagtgtgggccccaggacctgcgtgcaggcaggcaccctccccaaacccaccctctgggctgagccagcctctgtgatagctcgggggaagcccgtgaccctctggtgtcaggggcccctggagactgaggagtaccgtctggataaggagggactcccatgggcccggaagagacagaacccactggagcctggagccaaggccaagttccacattccatccacggtgtatgacagtgcagggcgataccgctgctactatgagacccctgcaggctggtcagagcccagtgaccccctggagctggtggcgacaggattctatgcagaacccactcttttagccctgccgagtcctgtggtggcctcaggaggaaatgtgaccctccagtgtgatacactggacggacttctcacgtttgttcttgttgaggaagaacagaagctccccaggaccctgtactcacagaagctccccaaagggccatcccaggccctgttccctgtgggtcccgtgacccccagctgcaggtggaggttcagatgctattactattacaggaaaaaccctcaggtgtggtcgaaccccagtgacctcctggagattctggtcccaggcgtgtctaggaagccctccctcctgatcccgcagggctctgtcgtggcccgcggaggcagcctgaccctgcagtgtcgctctgatgtcggctatgacatattcgttctgtacaaggagggggaacatgacctcgtccagggctctggccagcagccccaggctgggctctcccaggccaacttcaccctgggccctgtgagccgctcccacgggggccagtacagatgctacggtgcacacaacctctcccctaggtggtcggcccccagcgaccccctggacatcctgatcgcaggactgatccctgacatacccgccctctcggtgcagccgggccccaaggtggcctcaggagagaacgtgaccctgctgtgtcagtcatggcatcagatagacactttctttttgaccaaggagggggcagcccatcccccgctgtgtctaaagtcaaagtaccagtcttatagacaccaggctgaattctccatgagtcctgtgacctcagcccagggtggaacctaccgatgctacagcgcaatcaggtcctacccctacctgctgtccagccctagttacccccaggagctcgtggtctcaggaccctctggggatcccagcctctcacctacaggctccacccccacacctgcaggccctgaggaccagcccctcacccccacggggttggatccccagagtggtctgggaaggcacctgggggttgtgactggggtctcagtggccttcgtcctgctgctgttcctcctcctcttcctcctcctccgacatcggcatcagagcaaacacaggacatcggcccatttctaccgtcctgcaggggctgcggggccagagcccaaggaccagggcctgcagaagagggccagcccagttgctgacatccaggaggaaattctcaatgctgccgtgaaggacacacagcccaaggacggggtggagatggatgctcgggctgctgcatctgaagccccccaggatgtgacctacgcccagctacacagcttgaccctcagacgggaggcaactgagcctcctccatcccaggaaagggaacctccagctgaacccagcatctacgcccccctggccatccactagcccacgggggacccagatctcatactcaacagaaggagactcagagactccagaaggcacaggagctgcccccagtggacaccaatgaaccccagccagcctggacccctaacaaagaccaccaggacatcctgggaactctgggactcactagattctgcagtcaaagatgactaatatccttgcatttttgaaatgaagccacagacttctcaataaatcaatgagctgagaaaactgaaacagaaattagagcatggtataaatttggaatgataatgtaaatattacacattaaatgatgaaatcggaaaactacaaatgagcgaatgaattagaaaagaataaaacctacgtaattaatgaccttggcaatgacaaaaaaaaaaaaaaaa

(19) United States
(12) Patent Application Publication
(10) Pub. No.: US 2018/0177847 A1
(43) Pub. Date: Jun. 28, 2018

(54) COMPOSITIONS AND METHODS FOR MODULATING PRO-INFLAMMATORY IMMUNE RESPONSE

(71) Applicants: The Board of Regents of the University of Texas System, Austin, TX (US); Icahn School of Medicine at Mount Sinai, New York, NY (US)

(72) Inventors: Shu-Hsia CHEN, New York, NY (US); Chengcheng ZHANG, Dallas, TX (US); Xunlei KANG, Columbia, MO (US); Junke ZHENG, Shanghai (CN); Masato UMIKAWA, Okinawa (JP)

(21) Appl. No.: 15/834,004

(22) Filed: Dec. 6, 2017

Related U.S. Application Data
(63) Continuation of application No. 14/403,703, filed on Nov. 25, 2014.
(60) Provisional application No. 61/653,337, filed on May 30, 2012.

(51) Int. Cl. A61K 38/18; G01N 33/68; A61K 31/713; A61K 35/15; A61K 38/07; A61K 38/16; A61K 39/395; A61K 45/06; A61K 31/7105; C07K 16/28

(52) U.S. Cl. CPC A61K 38/1891; G01N 2800/7095; A61K 31/713; A61K 35/15; A61K 38/07; A61K 38/16; A61K 39/3955; A61K 45/06; A61K 31/7105; C07K 16/2803; G01N 2333/515; G01N 2333/70503; G01N 2500/04; G01N 2800/24; G01N 2800/245; G01N 2800/7028; G01N 33/6872

(57) ABSTRACT

Provided herein are compositions and methods useful for increasing a pro-inflammatory immune response, treating an autoimmune disorder, inflammation, or transplant rejection in a mammal by activating a leukocyte immunoglobulin-like receptor (LILR) protein. Also provided are compositions and methods useful for increasing a pro-inflammatory immune response, treating cancer, and treating infectious disease in a mammal by blocking the activation of a LILR protein.

### CROSS-REFERENCE TO RELATED APPLICATIONS

This application claims the benefit of U.S. Provisional Patent Application No. 61/653,337, filed on May 30, 2012. The contents of the foregoing is incorporated herein by reference in its entirety.

### BACKGROUND OF THE INVENTION

Myeloid-derived suppressor cells (MDSCs) are myeloid progenitors with immune suppressive functions that call differentiated/polarized into Gr1⁺CD11b⁺CD115⁺Ly6C⁺ monocytic (M)-cells and Gr1¹CD11b¹Ly6G¹ granulocytic (G)-cells in mice (Gabrilovich et al., Cancer Res. 67:425, 2007; Huang et al., Cancer Res. 66:1123-1131, 2006). Human MDSCs are characterized as CD11b⁺CD14^{Low}CD33⁺ or Lin⁻HLA⁻DR^{Low-}CD33⁺ myeloid cells (Ostrand-Rosenberg et al., J. Immunol. 182:4499-4506, 2009; Raychaudhuri et al., Neuro. Oncol. 13:591-599, 2011). In recent years, MDSCs have been found to play an important role in the regulation of the immune response in infection, malignancy, transplantation, and other immune disorders (e.g., Yin et al., J. Immunol. 185:5828-5834, 2010).

MDSCs can be differentiated and polarized into M1- and M2-cells (M1-cells expressing iNOS, TNF-α, IFN-gR, MHC class I, and CCR7, and M2-cells expressing arginase, IL-10, CD36, CD206, and CCR2). M2-cells possess an enhanced ability to suppress Teff activation and proliferation compared to their M1-like counterparts in co-cultures of T-cells and in vivo (Ma et al., Immunity 34:385-395, 2011). M2-cells also possess higher potency in Treg expansion than those with an M1 phenotype, both in vitro and in vivo (Ma et al., Immunity 34:385-395, 2011). As M2-cells suppress Teff activation and proliferation, and promote Treg expansion, M2-cells can be used to treat autoimmune diseases, where a decrease in pro-inflammatory immune response is desired.

M1-cells have increased direct tumor killing and promote the development of anti-tumoral immunity through the augmentation of free radicals, death ligand, and immunostimulating cytokines (see, e.g., Ma et al., Immunity 34:385-395, 2011), and therefore, M1-cells can be used to treat cancer or other disorders where an increase in pro-inflammatory immune response is desired.

### SUMMARY OF THE INVENTION

The invention is based, in part, on the discovery that glatiramer acetate and angiopoietin-like proteins bind and activate leukocyte immunoglobulin (Ig)-like receptor (LILR) B1 (LILRB1), LILRB2, LILRB3, LILRB4, and LILRB5 signaling that can modulate myeloid-derived suppressor cell differentiation/polarization. Based on this discovery, provided herein are compositions containing a LILRB1 agonist, a LILRB2 agonist, a LILRB3 agonist, a LILRB4 agonist, or a LILRB5 agonist and, optionally, one or more additional agents selected from the group of a myeloid-derived suppressor cell (MDSC), a mobilizing agent, a c-jun N-terminal kinase (JNK) inhibitor, an anti-inflammatory agent, and an immunosuppressive agent, and compositions containing one or more of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, or LILRB5 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, or LILRB5 mRNA in a mammalian cell; a soluble LILRB1 protein, a soluble LILRB2 protein, a soluble LILRB3 protein, a soluble LILRB4 protein, or a soluble LILRB5 protein; and, optionally, one or both of a chemotherapeutic agent and an analgesic. Also provided are methods of decreasing a pro-inflammatory immune response and treating an autoimmune disorder, inflammation, or transplant rejection in a mammal. Also provided are methods of increasing a pro-inflammatory immune response and treating cancer in a mammal, and methods for identifying candidate agents useful for treating inflammation, autoimmune disease, transplant rejection (e.g., graft-versus-host disease), infectious disease, or cancer in a mammal.

Provided herein are compositions containing a leukocyte immunoglobulin-like receptor (LILR) B1 (LILRB1) agonist, LILRB2 agonist, LILRB3 agonist, LILRB4 agonist, or LILRB5 agonist and one or more additional agents selected from the group of: a myeloid-derived suppressor cell, a mobilizing agent, a c-jun N-terminal kinase inhibitor, an anti-inflammatory agent, and an immunosuppressive agent. In some embodiments, the LILRB1 agonist, LILRB2 agonist, LILRB3 agonist, LILRB4 agonist, or LILRB5 agonist is glatiramer acetate. In some embodiments, the LILRB1 agonist, LILRB2 agonist, LILRB3 agonist, LILRB4 agonist, or LILRB5 agonist is an angiopoietin-like (Angptl)-1 protein, an Angptl-2 protein, an Angptl-3 protein, an Angptl-4 protein, an Angptl-5 protein, an Angptl-6 protein, or an Angptl-7 protein. In some embodiments, the LILRB1 agonist, LILRB2 agonist, LILRB3 agonist, LILRB4 agonist, or LILRB5 agonist is an endogenous Angptl-1 protein, Angptl-2 protein, Angptl-3 protein, Angptl-4 protein, Angptl-5 protein, Angptl-6 protein, or Angptl-7 protein. In some embodiments, the LILRB1 agonist, LILRB2 agonist, LILRB3 agonist, LILRB4 agonist, or LILRB5 agonist is an Angptl-5 protein. In some embodiments, the composition is formulated for intravenous, intramuscular, oral, subcutaneous, intraperitoneal, intrathecal, or intramuscular administration.

Also provided are compositions containing: one or more of an agent that specifically binds to an endogenous Angptl -1 protein, Angptl-2 protein, Angptl-3 protein, Angptl-4 protein, Angptl-5 protein, Angptl-6 protein, Angptl-7 protein, leukocyte immunoglobulin-like receptor (LILR) B1 (LILRB1) protein, LILRB2 protein, LILRB3 protein, LILRB4 protein, or LILRB5 protein; an oligonucleotide that decreases the expression of Angptl -1, Angptl-2, Angptl-3, Angptl-4, Angptl-5, Angptl-6, Angptl-7, LILRB1, LILRB2, LILRB3, LILRB4, or LILRB5 mRNA in a mammalian cell; and a soluble LILRB1 protein, soluble LILRB2 protein, soluble LILRB3 protein, soluble LILRB4 protein, or soluble LILRB5 protein; and one or both of a chemotherapeutic agent and an analgesic. In some embodiments, the agent that specifically binds to an endogenous Angptl -1 protein, Angptl-2 protein, Angptl-3 protein, Angptl-4 protein, Angptl-5 protein, Angptl-6 protein, Angptl-7, LILRB1, LILRB2, LILRB3, LILRB4, or LILRB5 protein is an antibody or an antigen-binding antibody fragment. In some embodiments, the agent that specifically binds to Angptl-1 protein, Angptl-2 protein, Angptl-3 protein, Angptl-4 protein, Angptl-5 protein, Angptl-6 protein, Angptl-7, LILRB1, LILRB2, LILRB3, LILRB4, or LILRB5 protein is an aptamer. In some embodiments, the oligonucleotide is an inhibitory RNA, an antisense RNA, or a ribozyme. In some embodiments, the inhibitory RNA is a small interfering RNA (siRNA). In some embodiments, the composition is formulated for intravenous, intramuscular, oral, subcutaneous, intraperitoneal, intrathecal, or intramuscular administration.

Also provided are methods of decreasing a pro-inflammatory immune response in a mammal that include administering to the mammal a therapeutically effective amount of a leukocyte immunoglobulin-like receptor (LILR) B1 (LILRB1) agonist, LILRB2 agonist, LILRB3 agonist, LILRB4 agonist, or LILRB5 agonist. In some embodiments, the LILRB1 agonist, LILRB2 agonist, LILRB3 agonist, LILRB4 agonist, or LILRB5 agonist is glatiramer acetate. In some embodiments, the LILRB1 agonist, LILRB2 agonist, LILRB3 agonist, LILRB4 agonist, or LILRB5 agonist is an angiopoietin-like (Angptl)-1 protein, an Angptl-2 protein, an Angptl-3 protein, an Angptl-4 protein, an Angptl-5 protein, an Angptl-6 protein, or an Angptl-7 protein. In some embodiments, the LILRB1 agonist, LILRB2 agonist, LILRB3 agonist, LILRB4 agonist, or LILRB5 agonist is an endogenous Angptl-1 protein, Angptl-2 protein, Angptl-3 protein Angptl-4 protein, Angptl-5 protein, Angptl-6 protein, or Angptl-7 protein. In some embodiments, the LILRB1 agonist, LILRB2 agonist, LILRB3 agonist, LILRB4 agonist, or LILRB5 agonist is an Angptl-5 protein. Some embodiments further include administering to the mammal one or more of a myeloid-derived suppressor cell, a mobilizing agent, a c-jun N-terminal kinase inhibitor, an anti-inflammatory agent, and an immunosuppressive agent. In some embodiments, the mammal is diagnosed as having inflammation, an autoimmune disease, or transplant rejection.

Also provided are methods of treating inflammation, an autoimmune disease, or transplant rejection in a mammal that include administering to the mammal a therapeutically effective amount of a leukocyte immunoglobulin-like receptor (LILR) B1 (LILRB1) agonist, a LILRB2 agonist, LILRB3 agonist, LILRB4 agonist, or LILRB5 agonist. In some embodiments, the LILRB1 agonist, LILRB2 agonist, LILRB3 agonist, LILRB4 agonist, or LILRB5 agonist is glatiramer acetate. In some embodiments, the LILRB1 agonist, LILRB2 agonist, LILRB3 agonist, LILRB4 agonist, or LILRB5 agonist is an angiopoietin-like (Angptl)-1 protein, an Angptl-2 protein, an Angptl-3 protein, an Angptl-4 protein, an Angptl-5 protein, an Angptl-6 protein, or an Angptl-7 protein. In some embodiments, the LILRB1 agonist, LILRB2 agonist, LILRB3 agonist, LILRB4 agonist, or LILRB5 agonist is an endogenous Angptl-1 protein, Angptl-2 protein, Angptl-3 protein, Angptl-4 protein, Angptl-5 protein, Angptl-6 protein, or Angptl-7 protein. In some embodiments, the LILRB1 agonist, LILRB2 agonist, LILRB3 agonist, LILRB4 agonist, or LILRB5 agonist is an Angptl-5 protein. Some embodiments further include administering to the mammal one or more of a myeloid-derived suppressor cell, a mobilizing agent, a c-jun N-terminal kinase inhibitor, an anti-inflammatory agent, and an immunosuppressive agent. In some embodiments, the mammal is diagnosed as having inflammation, an autoimmune disease, or transplant rejection. In some embodiments, the mammal is selected for organ or tissue transplantation.

Also provided are methods of stimulating a pro-inflammatory immune response in a mammal that include administering to the mammal a therapeutically effective amount of at least one of an agent that specifically binds to an endogenous angiopoietin-like (Angptl)-1 protein, Angptl-2 protein, Angptl-3 protein, Angptl-4 protein, Angptl-5 protein, Angptl-6 protein, Angptl-7 protein, leukocyte immunoglobulin-like receptor (LILR) B1 (LILRB1) protein, LILRB2 protein, LILRB3 protein, LILRB4 protein, or LILRB5 protein; an oligonucleotide that decreases the expression of Angptl -1, Angptl-2, Angptl-3, Angptl-4, Angptl-5, Angptl-6, Angptl-7, LILRB1, LILRB2, LILRB3, LILRB4, or LILRB5 mRNA in a mammalian cell; a soluble LILRB1 protein, a soluble LILRB2 protein, a soluble LILRB3 protein, a soluble LILRB4 protein, or a soluble LILRB5 protein. In some embodiments, the agent that specifically binds to an endogenous Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, or LILRB5 protein is an antibody or an antigen-binding antibody fragment. In some embodiments, the agent that specifically binds to an endogenous Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, or LILRB5 protein is an aptamer. In some embodiments, the oligonucleotide is an inhibitory RNA, an antisense RNA, or a ribozyme. In some embodiments, the inhibitory RNA is a small interfering RNA (siRNA). In some embodiments, the mammal is diagnosed as having a cancer. Some embodiments further include administering to the mammal a chemotherapeutic agent or an analgesic.

Also provided are methods of treating cancer in a mammal that include administering to the mammal a therapeutically effective amount of at least one of: an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, soluble LAIR1, soluble LATR2 protein, and/or an agent(s) that inhibits the signaling pathway(s) in cells (e.g., cancer cell(s)) initiated by Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., small molecules that inhibit SHP-1, SHP-2, CAMKI, CAMKII, or CAMKIV). In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is an antibody or an antigen-binding antibody fragment. In some embodiments, the agent that specifically binds to an endogenous Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LA1R2 protein is an aptamer. In some embodiments, the oligonucleotide is an inhibitory RNA, an antisense RNA, or a ribozyme. In some embodiments, the inhibitory RNA is a small interfering RNA (siRNA). In some embodiments, the mammal is diagnosed as having a cancer. Some embodiments further include administering to the mammal a chemotherapeutic agent or an analgesic.

Also provided are methods of using a LILRB1 agonist, LILRB2 agonist, LILRB3 agonist, LILRB4 agonist, or LILRB5 agonist in the manufacture of a medicament for decreasing a pro-inflammatory immune response in a mammal. Also provided herein are LILRB1 agonists, LILRB2 agonists, LILRB3 agonists, LILRB4 agonists, and/or LILRB5 agonists for use in decreasing a pro-inflammatory immune response in a mammal.

Also provided are methods of using a LILRB1 agonist, LILRB2 agonist, LILRB3 agonist, LILRB4 agonist, or LILRB5 agonist in the manufacture of a medicament for treating inflammation, an autoimmune disease, or transplant rejection in a mammal Also provided herein are LILRB1 agonists, LILRB2 agonists, LILRB3 agonists, LILRB4 agonists, and/or LILRB5 agonists for use in treating inflammation, an autoimmune disease, or transplant rejection in a mammal.

Also provided are methods of using at least one of: an agent that specifically binds to an Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, or LILRB5 protein; an oligonucleotide that decreases the expression of Angptl -1, Angptl-2, Angptl-3, Angptl-4, Angptl-5, Angptl-6, Angptl-7, LILRB1, LILRB2, LILRB3, LILRB4, or LTLRB5 mRNA in a mammalian cell; and a soluble LILRB1 protein, a soluble LILRB2 protein, a soluble LILRB3 protein, a soluble LILRB4 protein, or a soluble LILRB5 protein in the manufacture of a medicament for stimulating a pro-inflammatory immune response in a mammal. Also provided herein are Angptl-1 proteins, Angptl-2 proteins, Angptl-3 proteins, Angptl-4 proteins, Angptl-5 proteins, Angptl-6 proteins, Angptl-7 proteins, LILRB1 proteins, LILRB2 proteins, LILRB3 proteins, LILRB4 proteins, LILRB5 proteins; oligonucleotides that decreases the expression of Angptl-1, Angptl-2, Angptl-3, Angptl-4, Angptl-5, Angptl-6, Angptl-7, LILRB1, LILRB2, LILRB3, LILRB4, or LILRB5 mRNA in a mammalian cell; soluble LILRB1 proteins, soluble LILRB2 proteins, soluble LILRB3 proteins, soluble LILRB4 proteins, and soluble LILRB5 proteins for use in stimulating a pro-inflammatory immune response in a mammal.

Also provided are methods of using at least one of: an agent that specifically binds to an Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5, LAIR1, or LAIR2 protein; an oligonucleotide that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; and a soluble LILRB1 protein, a soluble LILRB2 protein, a soluble LILRB3 protein, a soluble LILRB4 protein, a soluble LILRB5 protein, a soluble LAIR1, a soluble LAIR2 protein, and/or an agent(s) that inhibits the signaling pathway(s) in cells (e.g., cancer cells) initiated by Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., small molecules that inhibit SHP-1, SHP-2, CAMKI, CAMKII, or CAMKIV) in the manufacture of a medicament for treating cancer in a mammal. Also provided herein are Angptl-1 proteins, Angptl-2 proteins, Angptl-3 proteins, Angptl-4 proteins, Angptl-5 proteins, Angptl-6 proteins, Angptl-7 proteins, LILRB1 proteins, LILRB2 proteins, LILRB3 proteins, LILRB4 proteins, LILRB5 proteins, LAIR1 proteins, and LAIR2 proteins; oligonucleotides that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; soluble LILRB1 proteins, soluble LILRB2 proteins, soluble LILRB3 proteins, soluble LILRB4 proteins, soluble LILRB5 proteins, soluble LAIR1, soluble LAIR2 proteins, and agents that inhibit the signaling pathway(s) in cells (e.g., cancer cells) initiated by Angptl-1 protein, Angptl-2 protein, Angptl-3 protein, Angptl-4 protein, Angptl-5 protein, Angptl-6 protein, Angptl-7 protein, LILRB1 protein, LTLRB2 protein, LILRB3 protein, LILRB4 protein, LILRB5 protein, LA1R1 protein, or LA1R2 protein (e.g., small molecules that inhibit SHP-1, SHP-2, CAMKI, CAMKII, or CAMKIV) for use in treating cancer in a mammal.

Also provided are methods of identifying a candidate agent for treating an autoimmune disease, inflammation, or transplant rejection in a mammal that include: (a) contacting a leukocyte immunoglobulin-like receptor (LILR) B1 (LILRB1) protein, LILRB2 protein, LILRB3 protein, LILRB4 protein, LILRB5 protein, or a paired immunoglobulin-like receptor B (PIRB) protein with a test agent, and determining the amount of binding of the test agent to the LILRB1 protein, LILRB2 protein, LILRB3 protein, LILRB4 protein, LILRB5 protein, or PIRB protein; (b) determining whether the test agent activates LILRB1 protein, LILRB2 protein, LILRB3 protein, LILRB4 protein, LILRB5 protein, or PIRB protein signaling in a cell; and (c) selecting a test agent that binds to LILRB1 protein, LILRB2 protein, LILRB3 protein, LILRB4 protein, LILRB5 protein, or PIRB protein, and activates LILRB1 protein, LILRB2 protein, LILRB3 protein, LILRB4 protein, LILRB5 protein, or PIRB protein signaling in a cell, respectively, as a candidate agent for treating an autoimmune disease, inflammation, or transplant rejection in a mammal. In some embodiments, the LILRB1 protein, LILRB2 protein, LLRB3 protein, LILRB4 protein, LILRB5 protein, or PIRB protein in (a) is expressed on the surface of a cell. In some embodiments, the cell in (b) is a T-cell. In some embodiments, the cell is in (b) a myeloid-derived suppressor cell.

Also provided are methods of identifying a candidate agent useful for treating a cancer in a mammal that include: contacting an angiopoietin-like (Angptl)-1 protein, an

Angptl-2 protein, an Angptl-3 protein, an Angptl-4 protein, an Angptl-5 protein, an Angptl-6 protein, or an Angptl-7 protein with a test agent; determining whether the test agent binds to the Angptl-1 protein, the Angptl-2 protein, the Angptl-3 protein, the Angptl-4 protein, the Angptl-5 protein, the Angptl-6 protein, or the Angptl-7 protein; and selecting a test agent that specifically binds to the Angptl-1 protein, the Angptl-2 protein, the Angptl-3 protein, the Angplt-4 protein, the Angptl-5 protein, the Angptl-6 protein, or the Angptl-7 protein as a candidate agent for treating a cancer in a mammal and/or selecting a test agent that specifically inhibits the signaling pathway(s) in a cell (e.g., a cancer cell) initiated by the Angptl-1 protein, the Angptl-2 protein, the Angptl-3 protein, the Angplt-4 protein, the Angptl-5 protein, the Angptl-6 protein, the Angptl-7 protein, the LILRB1 protein, the LILRB2 protein, the LILRB3 protein, the LILRB4 protein, the LILRB5 protein, or the LATR1 protein (e.g., a test agent that specifically inhibits one or more of SHP-1, SHP-2, CAMKI, CAMKII, and CAMKIV) as a candidate agent for treating a cancer in a mammal.

Also provided are methods of identifying a candidate agent useful for treating a cancer in a mammal that include: contacting a cell (e.g., a cancer cell) with a test agent;

determining whether the test agent inhibits signaling pathway(s) in the cell initiated by the Angptl-1 protein, the Angptl-2 protein, the Angptl-3 protein, the Angplt-4 protein, the Angptl-5 protein, the Angptl-6 protein, the Angptl-7 protein, the LILRB1 protein, the LILRB2 protein, the LILRB3 protein, the LILRB4 protein, the LILRB5 protein, or the LAIR1 protein (e.g., a test agent that specifically inhibits one or more of SHP-1, SHP-2, CAMKI, CAMKII, and CAMKIV); and selecting a test agent that inhibits signaling pathway(s) in the cell initiated by the Angptl-1 protein, the Angptl-2 protein, the Angptl-3 protein, the Angplt-4 protein, the Angptl-5 protein, the Angptl-6 protein, the Angptl-7 protein, the LILRB1 protein, the LILRB2 protein, the LILRB3 protein, the LILRB4 protein, the LILRB5 protein, or the LAIR1 protein as a candidate agent for treating a cancer in a mammal.

Also provided herein are methods of using a LILRB1 agonist, LILRB2 agonist, LILRB3 agonist, LILRB4 agonist, or LILRB5 agonist in the manufacture of a medicament for treating inflammation, an autoimmune disease, or transplant rejection in a mammal

Also provided herein are methods of using an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1, or a soluble LAIR2 protein, and/or an agent(s) that inhibits the signaling pathway(s) in cells (e.g., cancer cells) initiated by Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., small molecules that inhibit SHP-1, SHP-2, CAMKI, CAMKII, or CAMKIV) in the manufacture of a medicament for treating cancer in a mammal.

Also provided herein are LILRB1 agonists, LILRB2 agonists, LILRB3 agonists,

LILRB4 agonists, or LILRB5 agonists for use in treating inflammation, autoimmune disease, or transplant rejection in a mammal.

Also provided are agents that specifically binds to an endogenous Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; oligonucleotides that decrease the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; soluble LILRB1 proteins, soluble LILRB2 proteins, soluble LILRB3 proteins, soluble LILRB4 proteins, soluble LILRB5 proteins, soluble LAIR1, or soluble LAIR2 proteins, and agents that inhibit the signaling pathway(s) in cells (e.g., cancer cells) initiated by Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., small molecules that inhibit SHP-1, SHP-2, CAMKI, CAMKII, or CAMKIV) useful for treating cancer in a mammal.

By the term “proinflammatory immune response” is meant an immune response that includes one or more of increased levels of pro-inflammatory cytokines (e.g., IL-6, IL-23, IL-17, IL-1α, IL-1β, and TNF-α) and/or increased levels of Teff cells.

By the term “leukocyte immunoglobulin (Ig)-like receptor B1” or “LILRB1” is meant a mammalian (e.g., human) LILRB1 protein or mRNA, or a LILRB1 protein or mRNA derived from a mammalian (e.g., human) LILRB1 protein or mRNA. Non-limiting examples of LILRB1 proteins and mRNA are described herein. Additional examples of LILRB1 proteins and mRNA are known in the art.

By the term “LILRB1 agonist” is meant an agent that specifically binds to LILRB1 protein and activates LILRB1 signaling pathways in a mammalian cell. Non-limiting examples of LILRB1 agonists are described herein. Examples of LILRB1 signaling pathways are described in the Examples (e.g., decreased NF-κB, STAT1, ERK and/or p38 phosphorylation, and/or increased CAMKI, CAMKII, CAMKIV, SHP-1, and/or SHP-2 phosphorylation, and/or decrease TNF-α and/or increase IL-10 and TGF-β expression and secretion).

By the term “leukocyte immunoglobulin (Ig)-like receptor B2” or “LILRB2” is meant a mammalian (e.g., human) LILRB2 protein or mRNA, or a LILRB2 protein or mRNA derived from a mammalian (e.g., human) LILRB2 protein or mRNA. Non-limiting examples of LILRB2 proteins and mRNA are described herein. Additional examples of LILRB2 proteins and mRNA are known in the art.

By the term “LILRB2 agonist” is meant an agent that specifically binds to LILRB2 protein and activates LILRB2 signaling pathways in a mammalian cell. Non-limiting examples of LILRB2 agonists are described herein. Examples of LILRB2 signaling pathways are described in the Examples (e.g., decreased NF-κB, STAT1, ERK, and/or p38 phosphorylation, and/or increased CAMKI, CAMKII, CAMKIV, SHP-1, and/or SHP-2 phosphorylation, and/or decrease TNF-α and/or increase IL-10 and TGF-β expression and secretion).

By the term “leukocyte immunoglobulin (Ig)-like receptor B3” or “LILRB3” is meant a mammalian (e.g., human) LILRB3 protein or mRNA, or a LILRB3 protein or mRNA derived from a mammalian (e.g., human) LILRB3 protein or mRNA. Non-limiting examples of LILRB3 proteins and mRNA are described herein. Additional examples of LILRB3 proteins and mRNA arc known in the art.

By the term “LILRB3 agonist” is meant an agent that specifically binds to LILRB3 protein and activates LILRB3 signaling pathways in a mammalian cell. Non-limiting examples of LILRB3 agonists are described herein. Examples of LILRB3 signaling pathways are described in the Examples (e.g., decreased NF-κB, STAT1, ERK and/or p38 phosphorylation, and/or increased CAMKI, CAMKII, CAMKIV, SHP-1, and/or SHP-2 phosphorylation, and/or decrease TNF-α and/or increase IL-10 and TGF-β expression and secretion).

By the term “leukocyte immunoglobulin (Ig)-like receptor B4” or “LILRB4” is meant a mammalian (e.g., human) LILRB4 protein or mRNA, or a LILRB4 protein or mRNA derived from a mammalian (e.g., human) LILRB4 protein or mRNA. Non-limiting examples of LILRB4 proteins and mRNA are described herein. Additional examples of LILRB4 proteins and mRNA are known in the art.

By the term “LILRB4 agonist” is meant an agent that specifically binds to LILRB4 protein and activates LILRB4 signaling pathways in a mammalian cell. Non-limiting examples of LILRB4 agonists are described herein. Examples of LILRB4 signaling pathways are described in the Examples (e.g., decreased NF-κB, STAT1, ERK and/or p38 phosphorylation, and/or increased CAMKI, CAMKII, CAMKIV, SHP-1, and/or SHP-2 phosphorylation, and/or decrease TNF-α and/or increase IL-10 and TGF-β expression and secretion).

By the term “leukocyte immunoglobulin (Ig)-like receptor B5” or “LILRB5” is meant a mammalian (e.g., human) LILRB5 protein or mRNA, or a LILRB5 protein or mRNA derived from a mammalian (e.g., human) LILRB5 protein or mRNA. Non-limiting examples of LILRB5 proteins and mRNA are described herein. Additional examples of LILRB5 proteins and mRNA are known in the art.

By the term “LILRB5 agonist” is meant an agent that specifically binds to LILRB5 protein and activates LILRB5 signaling pathways in a mammalian cell. Non-limiting examples of LILRB5 agonists are described herein. Examples of LILRB5 signaling pathways are described in the Examples (e.g., decreased NF-κB, STAT1, ERK and/or p38 phosphorylation, and/or increased CAMKI, CAMKII, CAMKIV, SHP-1, and/or SHP-2 phosphorylation, and/or decrease TNF-α and/or increase IL-10 and TGF-β expression and secretion).

By the term “LAIR1” is meant a mammalian (e.g., human) LAIR1 protein or mRNA, or a LAIR1 protein or mRNA derived from a mammalian (e.g., human) LAIR1 protein or mRNA. Non-limiting examples of LAIR1 proteins and mRNA are described herein. Additional examples of LAIR1 proteins and mRNA are known in the art.

By the term “LAIR1 agonist” is meant an agent that specifically binds to LAIR1 protein and activates LAIR1 signaling pathways in a mammalian cell. Non-limiting examples of LAIR1 agonists are described herein. Examples of LAIR1 signaling pathways are described in the Examples (e.g., decreased NF-κB, STAT1, ERK and/or p38 phosphorylation, and/or increased CAMKI, CAMKII, CAMKIV, SHP-1, and/or SHP-2 phosphorylation, and/or decrease TNF-α and/or increase IL-β and TGF-β expression and secretion).

By the term “LAIR2” is meant a mammalian (e.g., human) LAIR2 protein or mRNA, or a LAIR2 protein or mRNA derived from a mammalian (e.g., human) LAIR2 protein or mRNA. Non-limiting examples of LAIR2 proteins and mRNA are described herein. Additional examples of LAIR2 proteins and mRNA are known in the art. By the term “mobilizing agent” is meant a therapeutic agent that increases the release of myeloid-derived suppressor cells from the bone marrow of a mammal. Non-limiting examples of mobilizing agents are described herein. Additional examples of mobilizing agents are known in the art.

By the term “myeloid-derived suppressor cell” or “MDSCs” is meant a heterogenous population of cells of myeloid origin that include myeloid progenitors, immature macrophages, immature granulocytes, and immature dendritic cells. In mice, MDSCs are CD11b⁺Gr1⁺. In humans, MDSCs include CD11b⁺CD14^{Low}CD33⁺ or Lin⁻HLA⁻DR^{Low.}CD33⁺ myeloid cells. Additional subsets and activities of MDSCs are described further herein.

By the term “angiopoietin-like-1” or “Angptl-1” is meant a mammalian (e.g., human) Angptl-1 protein or mRNA, or an Angptl-1 protein or mRNA derived from a mammalian (e.g., human) Angptl-1 protein or mRNA. Non-limiting examples of Angptl-1 proteins and mRNAs are described herein. Additional examples of Angptl-1 proteins and mRNAs are known in the art.

By the term “angiopoietin-like-2” or “Angptl-2” is meant a mammalian (e.g., human) Angptl-2 protein or mRNA, or an Angptl-2 protein or mRNA derived from a mammalian (e.g., human) Angptl-2 protein or mRNA. Non-limiting examples of Angptl-2 proteins and mRNAs are described herein. Additional examples of Angptl-2 proteins and mRNAs are known in the art.

By the term “angiopoietin-like-3” or “Angptl-3” is meant a mammalian (e.g., human) Angptl-3 protein or mRNA, or an Angptl-3 protein or mRNA derived from a mammalian (e.g., human) Angptl-3 protein or mRNA. Non-limiting examples of Angptl-3 proteins and mRNAs are described herein. Additional examples of Angptl-3 proteins and mRNAs are known in the art.

By the term “angiopoietin-like-4” or “Angptl-4” is meant a mammalian (e.g., human) Angptl-4 protein or mRNA, or an Angptl-4 protein or mRNA derived from a mammalian (e.g., human) Angptl-4 protein or mRNA. Non-limiting examples of Angptl-4 proteins and mRNAs are described herein. Additional examples of Angptl-4 proteins and mRNAs are known in the art.

By the term “angiopoietin-like-5” or “Angplt-5” is meant a mammalian (e.g., human) Angptl-5 protein or mRNA, or an Angptl-5 protein or mRNA derived from a mammalian (e.g., human) Angptl-5 protein or mRNA. Non-limiting examples of Angptl-5 proteins and mRNAs are described herein. Additional examples of Angptl-5 proteins and mRNAs are known in the art.

By the term “angiopoietin-like-6” or “Angptl-6” is meant a mammalian (e.g., human) Angptl-6 protein or mRNA, or an Angptl-6 protein or mRNA derived from a mammalian (e.g., human) Angptl-6 protein or mRNA. Non-limiting examples of Angptl-6 proteins and mRNAs are described herein. Additional examples of Angptl-6 proteins and mRNAs are known in the art.

By the term “angiopoietin-like-7” or “Angplt-7” is meant a mammalian (e.g., human) Angptl-7 protein or mRNA, or an Angptl-7 protein or mRNA derived from a mammalian (e.g., human) Angptl-7 protein or mRNA. Non-limiting examples of Angptl-7 proteins and mRNAs are described herein. Additional examples of Angptl-7 proteins and mRNAs are known in the art.

Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is two graphs showing the production of IL-10 (left graph) and TNF-α (right graph) from MDSCs isolated from wild-type or PIR-B knockout mice after incubation with vehicle or GA for 24 hours. The levels of IL-10 and TNFα were determined using ELISA kits.

FIG. 2 is a flow cytometric data of MDSCs from from LLC-tumor bearing wild type or PIR-B knockout mice following pre-incubation with vehicle (PBS, −) or GA (50 μg/mL), washing with cold PBS, and staining with anti-PIR-B antibody (top row), GA-FITC (middle row), or Flag-tagged angptl-2 (angptl-2-Flag) plus anti-Flag-FITC antibody (bottom row).

FIG. 3 is a set of four graphs showing the concentration of TGF-β (top left), IL-10 (top right), IL-6 (bottom left), and TNF-α (bottom right) from wild type MDSCs following incubation with vehicle or GA in the absence or presence of LPS (100 ng/mL) for 30 hours. The cytokine levels were quantified using ELISA kits.

FIG. 4 is a graph showing the T-cell proliferation (CPM) in co-cultures containing splenocytes (SPL) from CD4 HA-TCR Tg mice and irradiated MDSCs purified from MCA26-bearing BALB/c mice at the incidated ratios, in the presence or absence of SP600125 (1.1 μg/mL), GA (5 μg/mL), or both, and HA peptide. [³H]-Thymidine incorporation was used to assess T-cell proliferation.

FIG. 5 is a set of flow cytometric data from cultures of splenocytes or splenocytes and MDCSs at a ratio of 4:1, incubated in the presence or absence of GA, SP600125, or both for five days. The data show the number of CD4⁺CD25⁺Foxp3⁺ Treg cells in the resulting cultures.

FIG. 6 is flow cytometric data showing different FLAG-tagged Angptl binding to LILRB2-transfected 293T cells.

FIG. 7 is flow cytometric data showing the binding of FLAG-tagged Angptl-2 to 293T cells transfected with CMV-mouse and human LAIR1, followed by staining with anti-FLAG-APC and anti-LAIR1-PE. Both mouse and human LAIR1 bind to Angptl-1 and Angptl-7 (upper and lower panel, respectively).

FIG. 8 is flow cytometric data showing the ability of FLAG-tagged Angptls to bind to LILRB2⁺ human cord blood mononuclear cells (measured using FACSAria).

FIG. 9 is a graph showing the relative binding of Angptls to LILRB2⁺ human cord blood cells as determined from flow cytometric data. Relative binding was shown as compared to control. The asterisk represents p<0.05 (n=3), and the error bars represent the standard error of the mean.

FIG. 10 is flow cytometric data showing the co-staining of CD34, CD38, CD90, and LILRB2 in human cord blood mononuclear cells (measured using FACSCalibur).

FIG. 11 is a graph showing the percentage of human cord blood Cd34⁺ cell repopulation following transplantation of human cord blood CD34⁺ cells infected with LILRB2 shRNA-encoding virus (KD) or control scrambled shRNA virus into sub-lethally irradiated NOD/SCID mice before or after culture for 10 days. SCF+TPO+Flt3L (STF) or STF+Angptl-5 (STFA5) were used in the culture. The data shown are the human donor repopulation after 2 months (n=5-11). The asterisks represents p<0.05. The error bars represent the standard error of the mean.

FIG. 12 is flow cytometric data showing the ability of FLAG-Angptl-2 or GST-Angptl-5-FLAG to bind to PirB-transfected 293T cells.

FIG. 13 is flow cytometric data showing the ability of Angptl-2 to bind to the extracellular domain of PirB, but not Tie-2 in the conditioned medium of co-transfected 293T cells.

FIG. 14 is a graph showing the percentage repopulation of input 250 Lin⁻Sca-1⁺Kit⁺CD34⁻Flk2⁻ BM HSCs from wild type or PirBTM donors (n=5) after 8 days. SCF, TPO, and FGF-1, with or without Angptl-2, were used in culture. The asterisk represents p<0.05. The error bars represent the standard error of the mean.

FIG. 15 is flow cytometric data showing the PirB expression on YFP⁺Mac-1⁺Kit⁺ AML cells.

FIG. 16 is a graph of the survival data of mice receiving MLL-AF9-infected wild type or PirBTM hematopoietic progenitors. The asterisk represents p<0.05 (n=15).

FIG. 17 is three images and three graphs showing the relative sizes of the spleen and liver, and the number of peripheral blood cells of the mice transplanted with WT MLL-AF9 cells and those with PirBTM MLL-AF9 cells at 28 days after transplantation (n=6).

FIG. 18 is flow cytometry data showing that PirBTM AML mice have decreased Mac-1⁺Kit⁻ cells and increased differentiated cells relative to mice transplanted with WT cells at 28 days after transplantation.

FIG. 19 is a set of six images showing the colony forming activity of wild type and PirBTM MLL-AF9⁺ BM cells. The typical morphology of WT and PirBTM CFUs are shown.

FIG. 20 is a graph showing CFU numbers for wild type and PirBTM MLL-AF9⁺ BM cells following a second replating (n=3).

FIG. 21 is flow cytometric data and graphs showing the LILRB expressions on HLA-DR^{Low}-CD33⁺ MDSC from 16 cancer patients. The MDSC were pre-treated with vehicle (−) or GA (50 μg/mL). After washing, the cells were stained with anti-LILRB2 or anti-LILRB4. The mean fluorescence intensities (MFI) of LILRB2 and LILRB4 on vehicle- and GA-pre-treated MDSCs were re-plotted into histograms.

FIG. 22 are graphs showing the levels of IL-10, TNF-α, and TGF-β in supernatants from cultures of sorted human MDSCs following treatment with or without GA (50 μg/mL) stimulation.

FIG. 23 is cytokine profiles secreted from MDSCs at various groups, in which anti-LILRB2 and anti-LILRB4 binding can be competed with GA at higher than >70%, 70-30%, or lower than <30% groups.

FIG. 24 is set of four graphs showing the effect of shRNA knockdown of LILRB1, LILBR2, LILRB3, LILRB4, or LILRB5 on the growth of four different human leukemia cells lines (MV4-11, THP1, 697, and RCH-AVC (A-D, respectively) over time (n=3); and a set of four survival curves of acute myeloid leukemia (AML) patients having higher (dark lines) or lower (light lines) than average (GADPH-normalized) expression of LILRB1, LILRB2, LILRB3, or LILRB4 (E) (n=186).

FIG. 25 is a graph showing the correlation of the GADPH-normalized expression levels of different ITIM-containing receptors with overall survival in AML patients (A) (n=165); a graph showing the effect of shRNA-induced knockdown of different ITIM-receptors on the growth of leukemia MV4-11 and NB4 cells (as measured 6 days post-infection) (B) (n=3); a graph showing the effect of treatment with shRNA targeting lair1 on the growth of leukemia cells (MV4-11 cells) (C) (n=3); a graph showing the percentages of GFP cells (MV4-11 cells infected with a virus designed to express GFP and either scrambled shRNA or shRNA for lair1) that located to the bone marrow, spleen, liver, and peripheral blood at 1 month after transplantation into NSG mice (D) (n=7); a survival curve of NSG mice injected with GFP primary human leukemia cells infected with scrambled siRNA- or shRNA for lair1-lentivirus over time (E) (n=9; p<0.01); and a graph showing the percentage of GFP¹ cells in the bone marrow of NSG mice at 4-months after transplantation with control or lair-1 knockdown primary human AML cells (n=3; **p<0.01, ***p<0.001).

FIG. 26 is a survival curve of AML subjects having higher or lower than average (GADPH-normalized) SHP-1 expression (n=186) (A); a graph showing the effect of treatment with three different shRNAs targeting shp-1 on the growth of MV4-11 leukemia cells (n=3) (B); a graph showing the percentage of apoptotic cells in a population of MV4-11 cells at different time points following infection with shRNA 698 (targeting shp-1) (n=3; *p<0.05, ***p<0.001) (C); a survival curve of mice transplanted with 3,000 cre or control-infected YFP BM cells that were collected from primary recipients and transplanted with SHP-1 knockout (Cre) or control (Ctrl) MLL-AF9 AML cells (n=10; p<0.0001) (D); a graph showing the comparison of the percentages of GFP AML cells in peripheral blood between control and shp-1 knockout (cre) mice described in D (n=10) (E); a graph showing the colony forming ability of knockout (cre) and control MLL-AF9 AML cells (n=3) (F); and a graph showing the effect of the SHP-1 inhibitor sodium stibogluconate on the colony forming activity of MLL-AF9 AML cells (n=9; *p<0.05) (G).

FIG. 27 is a set of four graphs showing the survival curves of AML subjects having higher or lower than average (GADPH-normalized) expression of CAMKI, CAMKID, CAMKIID, or CAMKIV (n=186) (A); a graph showing the effect of knowndown of CaMK1 and CAMKIV on the growth of human B-ALL U937 cells (*p<0.05) (B); a graph showing the effect of knockdown of CAMPK1 and CAMPKIV on the growth of human AML MV4-11 cells (*p<0.05) (C); and a graph showing the effect of CaMK inhibitor or CaMKK inhibitor on the colony forming activity of WT AML cells or PirBTM AML cells (STO represents STO609 (CaMKK inhibitor); KN represents KN93 (CamK inhibitor)) (n=3; *p<0.05).

FIG. 28 is a graph of the interferon-γ production by human PBMCs that were stimulated with coated OKT3 (1 μg/mL) and anti-CD28 (1 μg/mL) for 4 days, in the presence or absence of coated Angptl-5 (10% Angptl-5 transfected culture supernatant or control supernatant) (A); a graph showing the IL-10 and a graph showing the IL-4 production of sorted human MDSC CD33⁺CD11b⁺CD14⁺ cells stimulated with coated Angptl5 (10% supernatant) for 48 hours (**p<0.01, ***p<0.001) (B, left and right graphs respectively); a graph showing the IL-10 production in CD33⁺ human myeloid-derived suppressor cells

(MDSCs) that were purified and treated with control IgG or anti-LILRB1-4 at a final concentration of 5 μg/mL for 30 minutes, then seeded into a 96-well plate coated with 10% supernatant from 293T cells without (untreated) or transfected with Angptl-5 and incubated for 2 days (mean±SEM) (C).

FIG. 29 is a graph showing the percentage of cytotoxic killing of K562 cells mediated by purified CD33⁺ human MDSCs that had been incubated with control IgG or anti-LILRBs for 48 hours (A), and a graph showing the percentage cytolytic T-cell mediated U937 cell killing mediated by irradiated purified CD33⁺ MDSCs incubated with control IgG, anti-LILRB3, or anti-LILRB3 and anti-LILRB4 for 30 minutes, followed by co-culturing with MDSC-depleted peripheral blood mononuclear cells (PBMC) at a 1:1 ratio in the presence of OKT3 (1 μg/mL) and anti-CD28 (1 μg/mL) for 4 days, and then co-culturing the cell mixture with leukemia U937 cells at a ratio of 100:1 for 4 hours (B).

FIG. 30 is a graph showing the tumor burden over time in mice following subcutaneous inoculation of SCID mice with 5×10⁶ U937 leukemia cells on day 0, and intravenous injection with 100 micrograms of control IgG or anti-LILRB3 every 3 days (p=0.028; F=6.53; d.f.=1.7, 6.9, Huynh-Fedt method).

### DETAILED DESCRIPTION OF THE INVENTION

The invention is based, in part, on the discovery that glatiramer acetate and angiopoietin-like proteins bind and activate LILRB1, LILRB2, LILRB3, LILRB4, and LILRB5 signaling that modulates MDSC differentiation/polarization. Based on this discovery, provided herein are are compositions containing a LILRB1 agonist, LILRB2 agonist, LILRB3 agonist, LILRB4 agonist, and/or LILRB5 agonist and, optionally, one or more of a myeloid-derived suppressor cell, a mobilizing agent, a JI\IK inhibitor, an anti-inflammatory agent, and an immunosuppressive agent, and compositions containing one or more of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein LILRB4 protein, or LILRB5 protein; an oligonucleotide that decreases the expression of Angptl -1, Angptl-2, Angptl-3,

Angptl-4, Angptl-5, Angptl-6, Angptl-7, LILRB1, LILRB2, LILRB3, LILRB4, or LILRB5 mRNA in a mammalian cell; and a soluble LILRB1 protein, a soluble LILRB2 protein, a soluble LILRB3 protein, a soluble LILRB4 protein, a soluble LILRB5 protein; and optionally, one or both of a chemotherapeutic agent and an analgesic. Also provided are methods of decreasing a pro-inflammatory immune response and treating an autoimmune disorder, inflammation, or transplant rejection in a mammal. Also provided are methods of increasing a pro-inflammatory immune response and treating cancer or infectious disease in a mammal, and methods for identifying candidate agents useful for treating inflammation, autoimmune disease, transplant rejection, cancer, or infectious disease in a mammal. Various, non-limiting features of each aspect of the invention are described below.

### Compositions and Kits

Provided herein are compositions (e.g., pharmaceutical compositions) that contain a LILRB1 agonist, LILRB2 agonist, LILRB3 agonist, LILRB4 agonist, and/or LILRB5 agonist and, optionally, one or more of a myeloid-derived suppressor cell, a mobilizing agent, a JNK inhibitor, an anti-inflammatory agent, and an immunosuppressive agent. In some embodiments of any of the compositions described herein, the LILRB1 agonist, LILRB2 agonist, LILRB3 agonist, LILRB4 agonist, and/or LILRB5 agonist is not glatiramer acetate. Also provided are compositions containing one or more of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angplt-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1, a soluble LAIR2 protein, and/or an agent(s) that inhibits the signaling pathway(s) in cells (e.g., cancer cells) initiated by Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., small molecules that inhibit SHP-1, SHP-2, CAMKI, CAMKII, or CAMKIV), and, optionally, one or both of a chemotherapeutic agent and an analgesic. Various exemplary aspects of these compositions are described below. One or more of any of the various exemplary aspects of these compositions can be used in any combination.

### LILRB1, LILRB2, LILRB3, LILRB4, and LILRB5 Agonists

### Antibodies and Antibody Fragments

In some embodiments, the LILRB1, LILRB2, LILRB3, LILRB4, or LILRB5 agonist is an antibody or antigen-binding antibody fragment (e.g., any of the antibodies or antigen-binding antibody fragments described herein) that specifically binds to LILRB1, LILRB2, LILRB3, LILRB4, or LILRB5 (e.g., any of the exemplary LILRB1, LILRB2, LILRB3, LILRB4, or LILRB5 proteins described herein), and activates LILRB1, LILRB2, LILRB3, LILRB4, or LILRB5 signaling, respectively.

### Glatiramer Acetate

Glatiramer acetate (Teva Pharmaceuticals) is a random polymer of four amino acids found in myelin basic protein, namely, glutamic acid, lysine, alanine, and tyrosine. Glatiramer acetate is capable of binding and activating LILRB1 (e.g., LILRB1 expressed on the surface of a MDSC), LILRB2 (e.g., LILRB2 expressed on the surface of a MDSC), LILRB3 (e.g., LILRB3 expressed on the surface of a MDSC), LILRB4 (e.g., LILRB4 expressed on the surface of a MDSC), and LILRB5 (e.g., LILRB5 expressed on the surface of a MDSC). Exemplary methods for making glatiramer acetate are described in U.S. Patent Application Publication No. 2010/0036092 (herein incorporated by reference).

### Angiopoeitin-Like Proteins

Several angiopoeitin-like proteins have been found to bind to and activate LILRB1, LILRB2, LILRB3, LILRB4, and LILRB5. Angiopoietin-like proteins belong to a family of secreted proteins. In some embodiments, the composition can contain an Angptl-1, Angptl-2, Angptl-3, Angptl-4, Angptl-5, Angptl-6, or Angptl-7 protein. In some embodiments, the Angptl-1, Angptl-2, Angptl-3, Angptl-4, Angptl-5, Angptl-6, or Angptl-7 protein is an endogenous mammalian (e.g., human) protein. In some embodiments, the Angptl-1, Angptl-2, Angptl-3, Angptl-4, Angptl-5, Angptl-6, or Angptl-7 protein is at least 80% identical (e.g., at least 85%, 90%, 91%, 92%, 93%, 94%, 95%, 96%, 97%, 98%, 99%, or 100% identical) to endogenous mammalian (e.g., human) Angptl-1, Angptl-2, Angptl-3, Angptl-4, Angptl-5, Angptl-6, or Angptl-7 protein. For example, in some embodiments the Angptl protein is at least 80% identical (e.g., at least 85%, 90%, 91%, 92%, 93%, 94%, 95%, 96%, 97%, 98%, 99%, or 100% identical) to SEQ ID NO: 1, 3, 5, 7, 43, 45, or 47.

Non-limiting examples of endogenous Angptl-1, Angptl-2, Angptl-3, Angptl-4, Angptl-5, Angptl-6, and Angptl-7 proteins (e.g., precursor or mature proteins) are: mature human Angptl-1 protein (SEQ ID NO: 1); mature human Angptl-2 protein (SEQ ID NO: 3); human Angplt-3 protein (SEQ ID NO: 43) (mature protein, amino acids 20 to 460; signal sequence, amino acids 1-19); human Angplt-4 protein (SEQ TD NO: 45) (mature protein, amino acids 23-503; signal sequence, amino acids 1-22); precursor human Angptl-5 protein (SEQ ID NO: 5) (mature protein, amino acids 26-388; signal sequence, amino acids 1-25); precursor human Angplt-6 protein (SEQ ID NO: 47) (mature protein, amino acids 21-470; signal sequence, amino acids 1-20); and precursor human Angptl-7 protein (SEQ ID NO: 7) (mature protein, amino acids 27 to 346; signal sequence, amino acids 1-26).

In some embodiments, the Angptl-1, Angptl-2, Angptl-3, Angplt-4, Angptl-5, Angptl-6, or Angptl-7 protein is conjugated to a stabilizing moiety (e.g., a polymer (e.g., polyethylene glycol), serum albumin, or an antibody Fc domain).

Exemplary cDNAs encoding Angptl-1, Angptl-2, Angptl-3, Angptl-4, Angptl-5, Angptl-6, and Angptl-7 protein are listed below. Nucleic acid sequences (e.g., mRNA or cDNA) encoding an endogenous Angptl-1, Angptl-2, Angptl-3, Angptl-4, Angptl-5, Angptl-6, or Angptl-7protein can be used to produce a Angptl-1, Angptl-2, Angptl-3, Angptl-4, Angptl-5, Angptl-6, or Angptl-7 protein. In some embodiments, a nucleic acid sequence containing a sequence at least 80% identical (e.g., at least 85%, 90%, 91%, 92%, 93%, 94%, 95%, 96%, 97%, 98%, 99%, or 100% identical) to an endogenous mRNA encoding an Angptl-1, Angptl-2, Angptl-3, Angptl-4, Angptl-5, Angptl-6, or Angptl-7protein (e.g., SEQ ID NO: 2, 4, 6, 8, 44, 46, or 48) is used to produce the Angptl-1, Angptl-2, Angptl-3, Angptl-4, Angptl-5, Angptl-6, or Angptl-7 protein. Methods for generating a protein using molecular biology techniques are well known in the art.

Non-limiting examples of cDNAs encoding Angptl-1, Angptl-2, Angptl-3, Angptl-4, Angptl-5, Angptl-6, and Angptl-7 protein are: human Angptl-1 cDNA (SEQ ID NO: 2); human Angptl-2 cDNA (SEQ ID NO: 4); human Angptl-3 cDNA (SEQ ID NO: 44); human Angptl-4 cDNA (SEQ ID NO: 46); human Angptl-5 cDNA (SEQ ID NO: 6); human Angptl-6 cDNA (SEQ ID NO: 48); and human Angptl-7 cDNA (SEQ ID NO: 8)

Agents that Specifically Bind to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 Protein

### Antibodies and Antigen-Binding Antibody Fragments

Non-limiting examples of agents that bind to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein include antibodies and antigen-binding antibody fragments. In some embodiments, the antibody or antigen-binding antibody fragment specifically binds to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein. Non-limiting examples of antibodies that can bind to Angptl-1 protein are available from Abgent (M03 antibody) and Santa Cruz (sc-365146 antibody). Non-limiting examples of antibodies that can bind to Angptl-2 protein are available from Abcam (ab89162), Abcam (ab36014), and Santa Cruz (sc-73711). A non-limiting example of an antibody that can bind to Angptl-3 is available from Abcam (ab30364). A non-limiting example of an antibody that binds to Angptl-4 is available from LifeSpan Biosciences. Non-limiting examples of antibodies that can bind to Angptl-5 are available from Sigma-Aldrich (HPA038516) and Santa Cruz (sc-134258). A non-limiting example of an antibody that binds to Angptl-6 is available from Abeam (ab57850). A non-limiting example of an antibody that can bind to Angptl-7 is available from Sigma Aldrich (266-280). A non-limiting example of an antibody that can bind to LILRB1 is available from Abcam (ab95828). A non-limiting example of an antibody that can bind to LILRB2 is available from Abcam (ab56696). A non-limiting example of an antibody that can bind to LILRB3 is available from Abcam (ab61890). A non-limiting example of an antibody that can bind to LILRB4 is available from Abeam (ab129772). A non-limiting example of an antibody that can bind to LILRB5 is available from Abcam (ab121357).

Methods for determining the ability of an antibody or antigen-binding antibody fragment to bind to an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein may be performed using the methods described herein and methods known in the art. Non-limiting examples of such methods include competitive binding assays using antibodies known to bind to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein, such as enzyme-linked immunosorbent assays (ELISAs), BioCoRE®, affinity columns, immunoblotting, or protein array technology. In some embodiments, the binding activity of the antibody or antigen-binding antibody fragment is determined by contacting a purified Angptl-1, Angptl-2, Angptl-3, Angptl-4, Angptl-5, Angptl-6, or Angptl-7 protein (e.g., SEQ ID NO: 1, 3, 5, 7, 43, 45, or 47) or a peptide fragment thereof, or a purified LILRB1, LILRB2, LILRB3, LILRB4, or LILRB5 protein (e.g., any of the LILRB1, LILRB2, LILRB3, LILRB4, or LILRB5 proteins described herein) or a peptide fragment thereof, with the antibody or antigen-binding antibody fragment. In other embodiments, the binding activity of the antibody or antigen-binding antibody fragment is determined by contacting a purified LAIR1 or LAIR 2 protein (e.g., human LAIR1 (NCBI Accession No. NP_068352.1 (signal sequence, amino acids 1-21; mature protein, amino acids 22 to 270)), human LATR1 (NCBI Accession No. NP_002278.1 (signal sequence, amino acids 1-21; mature protein, amino acids 22 to 287)), human LAIR2 (NCBI Accession No. NP_067154.1 (signal sequence, amino acids 1 to 21; mature protein, amino acids 22 to 135)), or human LAIR2 (NCBI Accession No. NP_002279.2 (signal sequence, amino acids 1 to 21; mature protein, amino acids 22 to 152))), or a peptide fragment thereof.

In some embodiments, the antibody or antigen-binding antibody fragment binds to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein (e.g., human Angptl-1, Angptl-2, Angptl-3, Angplt-4, Angptl-5, Angptl-6, Angptl-7, LILRB1, LILRB2, LILRB3, LILRB4, LILRB5, LAIR1, or LAIR2 protein) with an K_{D} equal to or less than 1×10⁻⁷ M, a K_{D} equal to or less than 1×10⁻⁸ M, a K_{D} equal to or less than 5×10⁻⁸ M, a K_{D} equal to or less than 5×10⁻⁹ M, a K_{D} equal to or less than 2×10⁻⁹ M, or a K_{D} equal to or less than 1×10⁻⁹ M under physiological conditions (e.g., in phosphate buffered saline).

An antibody can also be a single-chain antibody (e.g., as described herein). An antibody can be a whole antibody molecule (e.g., a human, humanized, or chimeric antibody) or a multimeric antibody (e.g., a bi-specific antibody). An antibody or antigen-binding antibody fragment may be a variant (including derivatives and conjugates) of an antibody or an antigen-binding antibody fragment. An antibody or an antigen-binding antibody fragment may also be a multi-specific (e.g., bi-specific) antibody or antigen-binding antibody fragment. Examples of antibodies and antigen-binding antibody fragments include, but are not limited to: single-chain Fvs (scFvs), Fab fragments, Fab′ fragments, F(ab′)₂, disulfide-linked Fvs (sdFvs), Fvs, and fragments containing either a VL or a VH domain. A single chain Fv or scFv is a polypeptide containing at least one VL domain of an antibody linked to at least one VH domain of an antibody.

Antibodies useful in the present invention include, e.g., polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding antibody fragments thereof. The antibodies or antigen-binding antibody fragments can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antibody or antigen-binding antibody fragment is an IgG₁ antibody or antigen-binding fragment thereof. In other embodiments, the antibody or antigen-binding antibody fragment is an IgG₄ antibody or antigen-binding fragment thereof. Immunoglobulins may have both a heavy and light chain.

An isolated fragment of an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein (e.g., a fragment of a human Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to a protein that is immunogenic in the species to be immunized, e.g., keyhole limpet hemocyanin, serum albumin, bovine thyroglobulin, or soybean trypsin inhibitor using a bifunctional or derivitizing agent, for example, malimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals can be injected with the antigenic peptide or protein more than one time (e.g., twice, three times, or four times).

Exemplary Angptl-1, Angptl-2, Angptl-3, Angptl-4, Angptl-5, Angptl-6, or Angptl-7 proteins that may be used to generate polyclonal or monoclonal antibodies are described herein (e.g., SEQ ID NO: 1, 3, 5, 7, 43, 45, or 47). Exemplary LILRB1, LILRB2, LILRB3, LILRB4, or LILRB5 proteins that can be used to generate polyclonal or monoclonal antibodies are described herein. In some embodiments, a full-length Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein can be used or, alternatively, antigenic Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 peptide fragments can be used as immunogens. The antigenic peptide of a protein comprises at least 8 (e.g., at least 10, 15, 20, or 30) amino acid residues of the amino acid sequence of an Angptl-1, Angptl-2, Angplt-3, Angptl-4, Angptl-5, Angptl-6, or Angptl-7 protein (e.g., at least 8 amino acid residues of SEQ ID NO: 1, 3, 5, 7, 43, 45, or 47), or of a LILRB1, LILRB2, LILRB3, LILRB4, LILRB5, LAIR1, or LAIR2 protein, and encompasses an epitope of the Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein such that an antibody raised against the peptide forms a specific immune complex with the Angptl-1, Angptl-2, Angptl-3, Angptl-4, Angptl-5, Angptl-6, Angptl-7, LTLRB1, LILRB2, LILRB3, LILRB4, LILRB5, LAIR1, or LAIR2 protein.

An immunogen typically is used to prepare antibodies by immunizing a suitable mammal (e.g., human or transgenic animal expressing at least one human immunoglobulin locus). An appropriate immunogenic preparation can contain, for example, a recombinantly-expressed or a chemically-synthesized polypeptide (e.g., a human Angptl-1, Angptl-2, Angptl-3, Angptl-4, Angptl-5, Angptl-6, or Angptl-7 protein (e.g., SEQ ID NO: 1, 3, 5, 7, 43, 45, or 47), or LILRB1, LILRB2, LILRB3, LILRB4, LILRB5, LAIR1, or LAIR2 protein (e.g., any of the LILRB1, LILRB2, LILRB3, LILRB4, LILRB5, LAIR1, or LAIR2 protein sequences described herein) or a fragment of human Angptl-1, Angptl-2, Angptl-3, Angptl-4, Angptl-5, Angptl-6, or Angptl-7 protein (e.g., a fragment of SEQ ID NO: 1, 3, 5, 7, 43, 45, or 47), or a fragment of a human LILRB1, LILRB2, LILRB3, LILRB4, LILRB5, LAIR1, or LAIR2 protein (e.g., a fragment of any of the LILRB1, LILRB2, LILRB3, LILRB4, LILRB5, LAIR1, or LAIR2 proteins described herein). The preparation can further include an adjuvant, such as Freund's complete or incomplete adjuvant, or a similar immunostimulatory agent.

Polyclonal antibodies can be prepared as described above by immunizing a suitable mammal with a Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein, or an antigenic peptide thereof (e.g., a fragment of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein containing at least 8 amino acids) as an immunogen. The antibody titer in the immunized mammal can be monitored over time by standard techniques, such as with an enzyme-linked immunosorbent assay (ELISA) using the immobilized Angptl-1, Angptl-2, Angplt-3, Angptl-4, Angptl-5, Angptl-6, Angptl-7, LILRB1, LILRB2, LILRB3, LILRB4, LILRB5, LAIR1, or LAIR2 protein or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the mammal and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the polypeptide or epitope of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide or a peptide fragment containing the epitope of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening an antibody display library can be found in, for example, U.S. Pat. No. 5,223,409; WO 92/18619; WO 91/17271; WO 92/2079; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., EMBO J. 12:725-734, 1993.

In some embodiments of any of the methods described herein, the antibodies are human antibodies, humanized antibodies, or chimeric antibodies that contain a sequence from a human antibody (e.g., a human immunoglobulin constant domain and/or human immunoglobulin variable domain framework regions). Humanized antibodies are chimeric antibodies that contain a minimal sequence derived from non-human (e.g., mouse) immunoglobulin. In some embodiments, a humanized antibody is a human antibody that has been engineered to contain at least one complementary determining region (CDR) present in a non-human antibody (e.g., a mouse, rat, rabbit, or goat antibody). In some embodiments, the humanized antibody or fragment thereof can contain all three CDRs of a heavy chain of a non-human monoclonal antibody that binds to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein (e.g., human Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein) and all three CDRs of a light chain of a non-human monoclonal antibody that binds to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, protein (e.g., human Angptl-1, Angptl-2, Angptl-3, Angptl-4, Angptl-5, Angptl-6, Angptl-7, LTLRB1, LTLRB2, LTLRB3, LILRB4, LILRB5, LAIR1, or LAIR2 protein). In some embodiments, the framework region residues of the human immunoglobulin are replaced by corresponding non-human (e.g., mouse) antibody residues. In some embodiments, the humanized antibodies can contain residues which are not found in the human antibody or in the non-human (e.g., mouse) antibody. Methods for making a humanized antibody from a non-human (e.g., mouse) monoclonal antibody are known in the art. Additional non-limiting examples of making a chimeric (e.g., humanized) antibody are described herein.

In some embodiments, the antibodies are chimeric antibodies that contain a light chain immunoglobulin that contains the light chain variable domain of a non-human antibody (e.g., a mouse antibody) or at least one CDR of a light chain variable domain of a non-human antibody (e.g., a mouse antibody) and the constant domain of a human immunoglobulin light chain (e.g., human κ chain constant domain). In some embodiments, the antibodies are chimeric antibodies that contain a heavy chain immunoglobulin that contains the heavy chain variable domain of a non-human (e.g., a mouse antibody) or at least one CDR of a heavy chain variable domain of a non-human (e.g., a mouse antibody) and the constant domain of a human immunoglobulin heavy chain (e.g., a human IgG heavy chain constant domain). In some embodiments, the chimeric antibodies contain a portion of a constant (Fc domain) of a human immunoglobulin.

In some embodiments, the antibodies or antigen-binding fragments thereof can be multi-specific (e.g., multimeric). For example, the antibodies can take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers. Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxyl ate) and SATA (N-succinimidyl S-acethylthio-acetate) (available, for example, from Pierce Biotechnology, Inc., Rockford, Ill.) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the C_{H}3 domain of an antibody constant domain. In this method, one or more small amino acid side chains from the interface of the first antibody molecule arc replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers (see, for example, WO 96/27011).

Bi-specific antibodies include cross-linked or heteroconjugate antibodies. For example, one of the antibodies in the heteroconjugate can be coupled to avidin and the other to biotin. Heteroconjugate antibodies can also be made using any convenient cross-linking methods. Suitable cross-linking agents are well known in the art and are disclosed in U.S. Pat. No. 4,676,980, along with a variety of cross-linking techniques.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Additional methods have been developed to facilitate the direct recovery of Fab′-SH fragments from E. coli, which can be chemically coupled to form bi-specific antibodies. Shalaby et al. (J. Exp. Med. 175:217-225, 1992) describes the production of a fully-humanized bi-specific antibody F(ab′), molecule. Each Fab′ fragment was separately secreted from E. coli and subjected to direct chemical coupling in vitro to form the bi-specific antibody.

Additional techniques for making and isolating bi-specific antibody fragments directly from recombinant cell culture have also been described. For example, bi-specific antibodies have been produced using leucine zippers (Kostelny et al., J. Immunol. 148:1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The diabody technology described by Hollinger et al. (Proc. Natl. Acad. Sci. U.S.A. 90:6444-6448, 1993) is an additional method for making bi-specific antibody fragments. The fragments contain a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another method for making bi-specific antibody fragments by the use of single-chain Fv (sFv) dimers has been described in Gruber et al. (J. Immunol. 153:5368, 1994). Alternatively, the bi-specific antibody can be a “linear” or “single-chain antibody” produced using the methods described, for example, in Zapata et al. (Protein Eng. 8:1057-1062, 1995). In some embodiments the antibodies have more than two antigen-binding sites. For example, tri-specific antibodies can be prepared as described in Tutt et al. (J. Immunol. 147:60, 1991).

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., Clin. Immunol. 101:21-31, 2001, and Frigerio et al., Plant Physiol. 123:1483-1494, 2000). IgA dimers are naturally secreted into the lumen of mucosa-lined organs. This secretion is mediated through the interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., J. Immunol., 167:5185-192, 2001). ScFv dimers can also be formed through recombinant techniques known in the art. An example of the construction of scFv dimers is given in Goel et al. (Cancer Res. 60:6964-71, 2000). Antibody multimers may be purified using any suitable method known in the art, including, but not limited to, size exclusion chromatography.

Any of the antibodies or antigen-binding fragments described herein may be conjugated to a stabilizing molecule (e.g., a molecule that increases the half-life of the antibody or antigen-binding fragment thereof in a mammal or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibody or antigen-binding antibody fragment binds to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein and prevents Angptl-1, Angptl-2, Angptl-3, Angptl-4, Angptl-5, Angptl-6, or Angptl-7 binding to LILRB1, LILRB2, LILRB3, LILRB4, LILRB5, LAIR1, and/or LAIR2 (e.g., LILRB1, LILRB2, LILRB3, LILRB4, and/or LILRB5 on a MDSC).

### Aptamers

Additional examples of agents that bind to Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein are aptamers. An aptamer is an oligonucleotide or peptide that is capable of binding to a specific polypeptide target. Methods of generating and screening oligonucleotide or peptide aptamers are known in the art. Exemplary methods for generating and screening oligonucleotide or peptide aptamers are described in U.S. Patent Application Publication Nos. 2012/0115752, and 2012/0014875; U.S. Pat. No. 7,745,607; and WO09/053691 (each of which is incorporated herein by reference). Additional methods for generating and screening oligonucleotide and peptide aptamers are described in Hoon et al., BioTechniques 51:413-416, 2011; Dausse et al., J. Nanobiotechnology 9:25, 2011; Hasegawa et al., Biotechnol. Lett. 30:829-834, 2008; and Drabovich et al., Analytical Chem. 78:6330-6335, 2006. Additional methods for generating and selecting aptamers that bind to Angptl-1, Angptl-2, Angptl-3, Angptl-4, Angptl-5, Angptl-6, or Angptl-7 protein (e.g., SEQ ID NO: 1, 3, 5, 7, 43, 45, or 47), or LILRB1, LILRB2, LILRB3, LILRB4, LILRB5, LAIR1, or LAIR2 protein are known in the art.

Oligonucleotides that Decrease the Expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA

Non-limiting examples of oligonucleotides that can decrease the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell include inhibitory nucleic acids (e.g., small inhibitory nucleic acids (siRNA)), antisense oligonucleotides, and ribozymes. Exemplary aspects of these different oligonucleotides are described below.

### Antisense Oligonucleotides

Oligonucleotides that decrease the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA expression in a mammalian cell include antisense nucleic acid molecules, i.e., nucleic acid molecules whose nucleotide sequence is complementary to all or part of an mRNA based on the sequence of a gene encoding a Angptl-1, Angptl-2, Angptl-3, Angptl-4, Angptl-5, Angptl-6, or Angptl-7 protein (e.g., complementary to all or a part of SEQ ID NO: 2, 4, 6, 8, 44, 46, or 48), or a LILRB1, LILRB2, LILRB3, LILRB4, LILRB5, LAIR1, or LAIR2 protein (e.g., complementary to all or a part of any of the LILRB1, LILRB2, LILRB3, LILRB4, LILRB5, LAIR1, or LAIR2 mRNA sequences described herein). An antisense nucleic acid molecule can be antisense to all or part of a non-coding region of the coding strand of a nucleotide sequence encoding a Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense molecules to target an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 gene described herein. For example, a “gene walk” comprising a series of oligonucleotides of 15-30 nucleotides spanning the length of an Angptl-1, Angptl-2, Angptl-3, Angptl-4, Angptl-5, Angptl-6, Angptl-7, LILRB1, LILRB2, LILRB3, LTLRB4, LILRB5, LAIR1, or LAIR2 gene can be prepared, followed by testing for inhibition of expression of the Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridinc substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein can be prepared in vitro and administered to a mammal, e.g., a human. Alternatively, they can be generated in situ such that they hybridize with or bind to cellular mRNA and/or genomic DNA encoding an Angptl-1, Angptl-2, Angptl-3, Angptl-4, Angptl-5, Angptl-6, or Angptl-7 protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol 11 or pol 111 promoter. In some embodiments, the vector used to express the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell can be a lentivirus, a retrovirus, or an adenovirus vector.

An antisense nucleic acid molecule of the invention can be an a-anomeric nucleic acid molecule. An a-anomeric nucleic acid molecule forms specific double-stranded hybrids with complementary RNA in which, contrary to the usual, p-units, the strands run parallel to each other (Gaultier et al., Nucleic Acids Res. 15:6625-6641, 1987). The antisense nucleic acid molecule can also comprise a 2′-O-methylribonucleotide (Inoue et al., Nucleic Acids Res., 15:6131-6148, 1987) or a chimeric RNA-DNA analog (Inoue et al., FEBS Lett., 215:327-330, 1987).

Antisense molecules that are complementary to all or part of an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 gene are also useful for assaying expression of an Angptl-1, Angptl-2, Angptl-3, Angptl-4, Angptl-5, Angptl-6, Angptl-7, L1LRB1, LILRB2, LILRB3, LILRB4, LILRB5, LAIR1, or LAIR2 gene using hybridization methods known in the art. For example, the antisense molecule is labeled (e.g., with a radioactive molecule) and an excess amount of the labeled antisense molecule is hybridized to an RNA sample. Unhybridized labeled antisense molecule is removed (e.g., by washing) and the amount of hybridized antisense molecule measured. The amount of hybridized molecule is measured and used to calculate the amount of expression of the Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA. In general, antisense molecules used for this purpose can hybridize to a sequence from an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 gene under high stringency conditions such as those described herein. When the RNA sample is first used to synthesize cDNA, a sense molecule can be used. It is also possible to use a double-stranded molecule in such assays as long as the double-stranded molecule is adequately denatured prior to hybridization.

### Ribozymes

Also provided are ribozymes that have specificity for sequences encoding an Angptl-1, Angptl-2, Angptl-3, Angptl-4, Angptl-5, Angptl-6, or Angptl-7 protein described herein (e.g., specificity for an Angptl-1, Angptl-2, Angptl-3, Angptl-4, Angptl-5, Angptl-6, or Angptl-7 mRNA, e.g., specificity for SEQ ID NO: 2, 4, 6, 8, 44, 46, or 48) or for sequences encoding a LILRB1, LILRB2, LILRB3, LILRB4, LILRB5, LAIR1, or LAIR2 protein described herein (e.g., a sequence encoding a human LILRB1, LILRB2, LILRB3, LILRB4, LILRB5, LAIR1, or LAIR2 protein). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. A ribozyme having specificity for a nucleic acid molecule of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA (Cech et al. U.S. Pat. No. 4,987,071; and Cech et al., U.S. Pat. No. 5,116,742). Alternatively, an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA can be used to select a catalytic RNA having a specific ribonuclease activity from a pool of RNA molecules. See, e.g., Bartel and Szostak, Science, 261:1411-1418, 1993.

Also provided herein are nucleic acid molecules that form triple helical structures. For example, expression of an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 polypeptide can be inhibited by targeting nucleotide sequences complementary to the regulatory region of the gene encoding the Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 polypeptide (e.g., the promoter and/or enhancer) to form triple helical structures that prevent transcription of the gene in target cells. See generally Helene, Anticancer Drug Des. 6(6):569-84, 1991; Helene, Ann. N.Y. Acad. Sci., 660:27-36, 1992; and Maher, Bioassays, 14(12):807-15, 1992.

In various embodiments, nucleic acid molecules (e.g., nucleic acid molecules used to decrease expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell) can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al., Bioorganic & Medicinal Chem., 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. USA, 93: 14670-675, 1996.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, 1996, supra; or as probes or primers for DNA sequence and hybridization (Hyrup, 1996, supra; Perry-O'Keefe et al., Proc. Natl. Acad. Sci. USA, 93: 14670-675, 1996).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup,1996, supra). The synthesis of PNA-DNA chimeras can be performed as described in Hyrup,1996, supra, and Finn et al., Nucleic Acids Res., 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res., 24:3357-63, 1996). Alternatively, chimeric molecules can be synthesized with a 5′ DNA segment and a 3′ PNA segment (Peterser et al., Bioorganic Med. Chem. Lett., 5:1119-11124, 1975).

In some embodiments, the oligonucleotide includes other appended groups such as peptides (e.g., for targeting host cell receptors in vivo), or agents facilitating transport across the cell membrane (see, e.g., Letsinger et al., Proc. Natl. Acad. Sci. USA, 86:6553-6556, 1989; Lemaitre et al., Proc. Natl. Acad. Sci. USA, 84:648-652, 1989; WO 88/09810) or the blood-brain barrier (see, e.g., WO 89/10134). In addition, oligonucleotides can be modified with hybridization-triggered cleavage agents (see, e.g., Krol et al., Bio/Techniques, 6:958-976, 1988) or intercalating agents (see, e.g., Zon, Pharm. Res., 5:539-549, 1988). To this end, the oligonucleotide may be conjugated to another molecule, e.g., a peptide, hybridization triggered cross-linking agent, transport agent, hybridization-triggered cleavage agent, etc.

siRNA

Another means by which expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA can be decreased in mammalian cell is by RNA interference (RNAi). RNAi is a process in which mRNA is degraded in host cells. To inhibit an mRNA, double-stranded RNA (dsRNA) corresponding to a portion of the gene to be silenced (e.g., a gene encoding an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 polypeptide) is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev. 15:485-490, 2001, and Hammond et al., Nature Rev. Gen., 2:110-119, 2001).

RNA-mediated gene silencing can be induced in mammalian cells in many ways, e.g., by enforcing endogenous expression of RNA hairpins (see, Paddison et al., Proc. Natl. Acad. Sci. USA, 99:1443-1448, 2002) or, as noted above, by transfection of small (21-23 nt) dsRNA (reviewed in Caplen, Trends in Biotech., 20:49-51, 2002). Methods for modulating gene expression with RNAi are described, e.g., in U.S. Pat. No. 6,506,559 and U.S. Patent Publication No. 2003/0056235, which are hereby incorporated by reference.

Standard molecular biology techniques can be used to generate siRNAs. Short interfering RNAs can be chemically synthesized, recombinantly produced, e.g., by expressing RNA from a template DNA, such as a plasmid, or obtained from commercial vendors such as Dharmacon. The RNA used to mediate RNAi can include synthetic or modified nucleotides, such as phosphorothioate nucleotides. Methods of transfecting cells with siRNA or with plasmids engineered to make siRNA are routine in the art.

The siRNA molecules used to decrease expression of an Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA can vary in a number of ways. For example, they can include a 3′ hydroxyl group and strands of 21, 22, or 23 consecutive nucleotides. They can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand.

To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). Any siRNA can be used in the methods of decreasing Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA, provided it has sufficient homology to the target of interest (e.g., a sequence present in SEQ ID NO: 2, 4, 6, 8, 44, 46, or 48, or a sequence present in any of the exemplary LILRB1, LILRB2, LILRB3, LILRB4, LILRB5, LAIR1, or LAIR2 mRNAs described herein). There is no upper limit on the length of the siRNA that can be used (e.g., the siRNA can range from about 21 base pairs of the gene to the frill length of the gene or more (e.g., 30-40, 40-50, 50-60, 60-70, 70-80, 80-90, 90-100, 100-120, 120-140, 140-160, 160-180, or 180-200 base pairs).

### Soluble LILRB1, LILRB2, LILRB3, LILRB4, LILRB5, LAIR1, and LAIR2 Proteins

In some embodiments, the compositions can contain a soluble LILRB1 protein, soluble LILRB2 protein, soluble LILRB3 protein, soluble LILRB4 protein, soluble LILRB5 protein, a soluble LAIR1 protein, or a soluble LAIR2 protein (e.g., a soluble human LILRB1, soluble human LILRB2, soluble human LILRB3, soluble human LILRB4, soluble human LILRB5 protein, a soluble LAIR1 protein, or a soluble LAIR2 protein).

LILRB1 protein is a transmembrane protein that contains four extracellular immunoglobulin domains (e.g., amino acids 27-119, amino acids 124-209, amino acids 225-318, and amino acids 327-419 of SEQ ID NO: 55), a transmembrane domain (e.g., amino acids 462-482 of SEQ ID NO: 55), and an intracellular domain that contains four cytoplasmic immunorccpetor tyrosine-based inhibitory motifs (ITIM) (c.g., amino acids 531-536, amino acids 561-566, amino acids 613-618, and amino acids 643-648 of SEQ ID NO: 55). In some embodiments, the soluble LILRB1 protein is the extracellular domain of an endogenous mammalian LILRB1 protein (e.g., a protein containing amino acids 27-419 of SEQ ID NO: 55). In some embodiments, the soluble LILRB1 protein contains a sequence that is at least 80% identical to the extracellular domain of an endogenous mammalian LILRB1 protein (e.g., amino acids 27-419 of SEQ ID NO: 55).

LILRB2 protein is a transmembrane protein that contains four extracellular immunoglobulin domains (e.g., amino acids 27-118, amino acids 123-218, amino acids 224-317, and amino acids 324-418 of SEQ ID NO: 9), a transmembrane domain (e.g., amino acids 461-481 of SEQ ID NO: 9), and an intracellular domain that contains three cytoplasmic immunoreceptor tyrosine-based inhibitory motifs (ITIM) (e.g., amino acids 530-535, amino acids 559-564, and amino acids 589-594 of SEQ ID NO: 9). In some embodiments, the soluble LILRB2 protein is the extracellular domain of an endogenous mammalian LILRB2 protein (e.g., the extracellular domain of any of the LILRB2 proteins shown below, e.g., a protein containing amino acids 27-418 of SEQ ID NO: 9). In some embodiments, the soluble LILRB2 protein contains a sequence that is at least 80% identical to the extracellular domain of an endogenous mammalian LILRB2 protein (e.g., amino acids 27-418 of SEQ ID NO: 9).

LILRB3 protein is a transmembrane protein that contains four extracellular immunoglobulin domains (e.g., amino acids 27-118, amino acids 123-217, amino acids 224-316, and amino acids 326-418 of SEQ ID NO: 57), a transmembrane domain (e.g., amino acids 444-464 of SEQ ID NO: 57), and an intracellular domain that contains three cytoplasmic immunoreceptor tyrosine-based inhibitory motifs (TTTM) (e.g., amino acids 512-517, amino acids 594-599, and amino acids 624-629 of SEQ ID NO: 57). In some embodiments, the soluble LILRB3 protein is the extracellular domain of an endogenous mammalian LILRB3 protein (e.g., a protein containing amino acids 27-418 of SEQ ID NO: 57). In some embodiments, the soluble LILRB3 protein contains a sequence that is at least 80% identical to the extracellular domain of an endogenous mammalian LILRB3 protein (e.g., amino acids 27-418 of SEQ ID NO: 57).

LILRB4 protein is a transmembrane protein that contains two extracellular immunoglobulin domains (e.g., amino acids 27-118 and amino acids 123-217 of SEQ ID NO: 49), a transmembrane domain (e.g., amino acids 260-280 of SEQ ID NO: 49), and an intracellular domain that contains three cytoplasmic immunoreceptor tyrosine-based inhibitory motifs (ITIM) (e.g., amino acids 357-362, amino acids 409-414, and amino acids 439-444 of SEQ TD NO: 49). In some embodiments, the soluble LILRB4 protein is the extracellular domain of an endogenous mammalian LILRB4 protein (e.g., the extracellular domain of any of the LILRB4 proteins shown below, e.g., a protein containing amino acids 27-217 of SEQ ID NO: 49). In some embodiments, the soluble LILRB4 protein contains a sequence that is at least 80% identical to the extracellular domain of an endogenous mammalian LILRB4 protein (e.g., amino acids 27-217 of SEQ ID NO: 49).

LILRB5 protein is a transmembrane protein that contains four extracellular immunoglobulin domains (e.g., amino acids 27-118, amino acids 124-217, amino acids 223-316, amino acids 324-400 of SEQ ID NO: 59), a transmembrane domain (e.g., amino acids 460-480 of SEQ ID NO: 59), and an intracellular domain that contains two cytoplasmic immunoreceptor tyrosine-based inhibitory motifs (ITIM) (e.g., amino acids 553-558 and amino acids 583-588 of SEQ ID NO: 59). In some embodiments, the soluble LILRB5 protein is the extracellular domain of an endogenous mammalian LILRB5 protein (e.g., a protein containing amino acids 27-400 of SEQ ID NO: 59). In some embodiments, the soluble LILRB5 protein contains a sequence that is at least 80% identical to the extracellular domain of an endogenous mammalian LILRB5 protein (e.g., amino acids 27-400 of SEQ ID NO: 59).

LAIR1 is a type I transmembrane protein (e.g., transmembrane domain of amino acids 166 to 186 of NCBI Ref. No. NP_002278.1) with one Ig-like domain (e.g., amino acids 27 to 120 of NCBI Ref. No. NP_002278.1) and two cytoplasmic ITIM domains (amino acids 249 to 254, and amino acids 279 to 284 of NCBI Ref. No. NP_002278.1). In some embodiments, the soluble LAIR protein is the extracellular domain of an endogenous mammalian LAIR1 protein (e.g., a protein containing at least the extracellular domain of LAIR 1, e.g., amino acids 27-165 of NCBI Ref No. NP_068352.1, or amino acids 27 to 165 of NCBI Ref No. NP_002278.1). In some embodiments, the soluble LAIR1 protein contains a sequence that is at least 80% identical to the extracellular domain of an endogenous mammalian LAIR1 protein (e.g., amino acids 27-165 of NCBI Ref. No. NP_002278.1).

LAIR2 is a secreted protein that is about 83% identical to the extracellular domain of LAIR1. In some embodiments, soluble LAIR2 protein is a wildtype LAIR2 protein (e.g., NCBI Ref No. NP_067154.1 or NP_002279.2) In some embodiments, the soluble LAIR2 protein contains a sequence that is at least 80% identical to a wildtype LAIR2 protein (e.g., NCBI Ref No. NP_067154.1 or NP_002279.2).

Examples of mammalian LILRB1, LILRB2, LILRB3, LILRB4, and LILRB5 proteins and cDNAs are listed below. Examples of mammalian LAIR1 and LAIR2 cDNAs are also provided below. Additional mammalian LILRB1, LILRB2, LILRB3, LILRB4, LILRB5, LAIR1, and LAIR2 proteins and mRNAs are known in the art. Non-limiting examples of mammalian LILRB1, LILRB2, LILRB3, LILRB4, and LILRB5 proteins are: human LILRB1 protein (SEQ ID NO: 55) (extracellular domain, amino acids 24-419); human LILRB2 protein (SEQ ID NO: 9) (extracellular domain, amino acids 27-418); dog LILRB2 protein (SEQ ID NO: 11) (extracellular domain, amino acids 1-395); chimpanzee LILRB2 protein (SEQ ID NO: 13) (extracellular domain, amino acids 1-418); human LILRB3 Protein (SEQ ID NO: 57) (extracellular domain, amino acids 27-418; signal sequence, amino acids 1-26); human LILRB4 protein (SEQ ID NO: 49) (extracellular domain, amino acids 1-217); mouse LILRB4 protein (SEQ ID NO: 51) (extracellular domain, amino acids 1-218); rat LILRB4 protein (SEQ ID NO: 53)(extracellular domain, amino acids 1-218); human LILRB4 protein (SEQ ID NO: 59) (extracellular domain, amino acids 27-459); human LILRB1 cDNA (SEQ ID NO: 56); human LILRB2 cDNA (SEQ ID NO: 10); dog LILRB2 cDNA (SEQ ID NO: 12); chimpanzee LILRB2 cDNA (SEQ ID NO: 14); human LILRB3 cDNA (SEQ ID NO: 58); human LILRB4 cDNA (SEQ ID NO: 50); mouse LILRB4 cDNA (SEQ ID NO: 52); rat LILRB4 cDNA (SEQ ID NO: 54); and human LILRB4 cDNA (SEQ ID NO: 60).

Non-limiting examples of LAIR1 cDNAs include: human LAIR1 cDNA NCBI Ref. No. NM_021706.2 and human LAIR1 cDNA NCBI Ref No. NM_002287.3. Non-limiting examples of LAIR2 cDNAs include: human LAIR2 cDNA NCBI Ref. No. NM_002288.4 and human LAIR2 cDNA NCBI Ref. No. NM_021270.3.

In some embodiments, the soluble LILRB1, LILRB2, LILRB3, LILRB4, or LILRB5 protein is at least 400, 410, 420, 430, 440, 450, 460, 470, 480, 490, or 500 amino acids in length. In some embodiments, the soluble LATR1 or LATR2 protein is at least 50, 100, 110, 120, 130, 140, or 145 amino acids In some embodiments, the soluble LILRB1, LILRB2, LILRB3, LILRB4, LILRB5, LAIR1, or LAIR2 protein contains a stabilizing moiety that increases the half-life of the LILRB1, LILRB2, LILRB3, LILRB4, LILRB5, LAIR1, or LAIR2 protein in vivo or in vitro. Non-limiting examples of stabilizing moieties that can be conjugated to a sequence that is at least 80% identical (e.g., at least 85%, 90%, 91%, 92%, 93%, 94%, 95%, 96%, 97%, 98%, 99%, or 100% identical) to an extracellular domain of LILRB1, LILRB2, LILRB3, LILRB4, LILRB5, or LAIR1 protein (e.g., any of the mammalian LILRB1, LILRB2, LILRB3, LILRB4, LILRB5, or LAIR1 extracellular domains described herein) or to a LAIR2 protein (e.g., any of the mammalian LAIR2 proteins described herein) include a polymer (e.g., polyethylene glycol), serum albumin (e.g., human serum albumin), or an Fc domain of an antibody (e.g., an Fc region of a human antibody).

Agents that Inhibit Signaling Pathway(s) Initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2

Various signaling pathway(s) that are initiated by Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein are described herein. For example, signaling pathways that include SHP-1, SHP-2, CAMKI, CAMKII, and/or CAMKIV are initiated Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, and/or LAIR2 protein. The ability of an agent to inhibiting signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, and/or LAIR2 can be determined using any of the methods described herein (see, examples). The agent can be a protein, a peptide, a small molecule, a lipid, a carbohydrate, or any combination thereof. For example, the agent can be an inhibitor of SHP-1, SHP-2, CAMK1, CAMKII, and/or CAMKIV. Non-limiting examples of such agents include: NSC-87877, stibogluconate, TPI-1, CK59, ST0609, and KN93. Additional examples of inhibitors of SHP-1, SHP-2, CAMK1, CAMKII, and CAMKIV, and other proteins involved in the signaling pathways initiated by Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein (e.g., the signaling pathways described herein) are known in the art.

### Additional Agents

### MDSCs

MDSCs have recently been recognized as one of the central regulators of the immune system. MDSCs represent a heterogenous population of cells of myeloid origin that include myeloid progenitors, immature macrophages, immature granulocytes, and immature dendritic cells. MDSCs differentiate and polarize into Gr1⁺CD11b⁺CD115⁺Ly6C⁺ monocytic (M)-cells and Gr1⁻CD11b⁺Ly6G⁺ granulocytic (G)-cells in mice (Gabrilovich et al., Cancer Res. 67:425, 2007; Huang et al., Cancer Res. 66:1123-1131, 2006; Movahedi et al., Blood 111:4233-4244, 2008). Human MDSCs are characterized as CD11b⁺CD14^{Low}CD33⁺ or Lin⁻ HLA⁻DR^{Low}CD33⁺ myeloid cells (Ostrand-Rosenberg et al., J. Immunol. 182:4499-4506, 2009; Raychaudhuri et al., Neurol. Oncol.13:591-599, 2011). Mirroring the nomenclature of type 1 classical activation-like (M1) and type 2 alternative activation-like (M2) macrophages, MDSCs can be differentiated and polarized into M1- and M2-cells (M1-cells expressing iNOS, TNF-α, IFN-gR, MHC class I, and CCR7, and M2-cells expressing arginase, IL-10, CD36, CD206, and CCR2). Tumor-associated MDSCs exhibit predominantly M2-like phenotypes with pro-tumoral and immunosuppressive activities. M2-cells are phenotypically characterized by a number of enhanced signature markers such as IL-10, arginase, IL-10, Tie-2, CD36, CD206, IL-4R and CCR2 (Ma et al., Immunity 34:385-395, 2011). M1-cells have an elevation in the expression of iNOS, NO, TNF-α, IFN-γR, MHC I, and CCR7 (Ma et al., Immunity 34:385-395, 2011). G2-cells up-regulate the expression of arginase, CCL2, CCL5 and MMP-9. In contrast, G1-cells show elevated expression levels of TNF-α, Fas, and ICAM-1.

MDSCs exert immune suppression through cross-communication with T-cells, NK cells, dendritic cells, macrophages, and other immune cells via multiple mechanisms. The details of how MDSC cross-talk with other immune cells are described in Bunt et al. (J. Leukoc. Biol. 85:996-1004, 2009), Ostrand-Rosenberg et al. (Nat. Rev. Immunol. 12:253-268, 2012), and Sinha et al. (J. Immunol. 179:977-983, 2007). As far as T-cells are concerned, MDSCs can induce effector T-cell (Teff) inactivation and apoptosis (see, e.g., Apolloni et al., J. Immunol. 165:6723-6730, 2000) and expand regulatory T cells (Treg) (see, e.g., Adeegbe et al., Cell Transplant. 20:941-954, 2011). The regulation of T-cell suppression and Treg expansion by MDSC is cell contact-, MHC class II-, NO- and/or arginase-dependent. M2-cells possess an enhanced ability to suppress Teff activation and proliferation compared to their M1-like counterparts in co-cultures of T-cells (Ma et al., Immunity 34:385-395, 2011). M2-cells possess higher potency in Treg expansion than M1-cells, both in vitro and in vivo (Ma et al., Immunity 34:385-395, 2011). M2-cell-induced increase in Treg cells appears to be

IL-10-, IL-4-, and IL-13-mediated and arginase-dependent (Ma et al., Immunity 34:385-395, 2011) Akin to the functionalities of M1/M2 cells, G1- and G2-cells possess anti-tumoral and pro-tumoral activities, respectively (Fridlender et al., Cancer Cell 16:183-194, 2009).

Polarization of MDSC subsets from one phenotype to the other is accompanied by functional changes. M2-cells accelerate tumor growth mainly by enhanced immune suppression involving an increase in arginase and immunosuppressive cytokines (see, e.g., Ma et al., Immunity 34:385-395, 2011). M1-cells have increased direct tumor killing and promote the development of anti-tumoral immunity through the augmentation of free radicals, death ligand, and immunostimulating cytokines (see, e.g., Ma et al., Immunity 34:385-395, 2011). The balance of M1/M2 polarization may have a significant influence on disease and health. The data provided herein indicate that LILRB1, LILRB2, LILRB3, LILRB4, and LILRB5 agonists stimulate MDSC polarization to the M2 phenotype.

Methods of preparing and isolating MDSCs are known in the art. For example, MDSCs can be isolated using fluorescence-assisted cell sorting using antibodies that recognize any of the specific protein markers of the different MDSC subsets described herein. Exemplary methods for preparing and isolating MDSCs are described in U.S. Patent Application Publication No. 2008/0305079 and WO 11/087795 (each of which is herein incorporated by reference).

### Mobilizing Agents

In some embodiments, the compositions further contain one or more mobilizing agents. In some embodiments, a composition further containing a mobilizing agent does not include MDSCs. In some embodiments, a composition contains a mobilizing agent and at least one LILRB1, LILRB2, LILRB3, LILRB4, and/or LILRB5 agonist. In some embodiments, a composition contains a mobilizing agent, at least one LILRB1, LILRB2, LILRB3, LILRB4, and/or LILRB5 agonist, and at least one JNK inhibitor.

Mobilizing agents stimulate the release of MDSCs from the bone marrow of a mammal. Non-limiting examples of mobilizing agents include granulocyte colony stimulating factor (G-CSF), cyclophosphamide, AMD3100, Fms-like tyrosine kinase 3 ligand (F1t3-L), GM-CSF, M-CSF, IL-34, TSLP-1, SCF, FK560, S100 A8, and S100 A9.

JNK Inhibitors In some embodiments, the compositions further contain at least one JNK inhibitor. Non-limiting examples of JNK inhibitors include B1-78D3, SP600125, AEG 3482, J1P-1, SU 3327, TCS JNK 5a, and TCS JNK 6o. Additional examples of JNK inhibitors are described in WO 00/35906, WO 00/35909, WO 00/35921, WO 00/64872, WO 01/12609, WO 01/12621, WO 01/23378, WO 01/23379, WO 01/23382, WO 01/47920, WO 01/91749, WO 02/046170, WO 02/062792, WO 02/081475, WO 02/083648, and WO 03/024967, each of which are herein incorporated by reference.

### Anti-Inflammatory Agents

In some instances, the composition can also contain one or more anti-inflammatory agents. Anti-inflammatory agents include, e.g., corticosteroids, non-steroidal anti-inflammatory drugs (NSAIDs, e.g., cyclooxygenase I (COX I) inhibitors and cyclooxygenase II (COX-II) inhibitors), immune selective anti-inflammatory derivatives (ImSAIDs), and biologics. Any of the exemplary anti-inflammatory agents described herein or known in the art can be included in the compositions described herein.

Non-limiting examples of NSAIDs are salicylates (e.g., aspirin, diflusinal, and salsalate), propionic acid derivatives (e.g., ibuprofen, dexiboprofen, naproxen, fenoprofen, ketoprofen, dexketoprofen, flurbiprofen, oxaprozin, and loxoprofen), acetic acid derivatives (e.g., indomethacin, sulindac, etodolac, ketorolac, diclofenac, and nabumetone), enolic acid derivatives (e.g., piroxicam, meloxicam, tanoxicam, droxicam, lornoxicam, and isoxicam), fenamic acid derivatives (e.g., mefamic acid, meclofenamic acid, flufenamic acid, and tolfenamic acid), sulphonanilides (e.g., nimesulide), licofelone, and lysine clonixinate. In some embodiments, an NSAID is a COX-I inhibitor or a COX-II inhibitor. Non-limiting examples of COX-I inhibitors include aspirin, ibuprofen, and naproxen. Non-limiting examples of COX-II inhibitors include celecoxib, valdecoxib, and rofecoxib.

Non-limiting examples of ImSAIDs include FEG (Phe-Glu-Gly), its D-isomer feG, and SGP-T peptide. Non-limiting examples of corticosteroids include hydrocortisone, cortisone acetate, tixocortol pivalate, prednisolone, methylprednisolone, prednisone, triamcinolone acetonide, triamcinolone alcohol, mometasone, amcinonide, budesonide, desonide, fluocinolone, halcinonide, betamethasone, dexamethasone, and fluocortolone. Non-limiting examples of biologics include tocilizumab, certolizumab, etanercept, adalimumab, anakinra, abatacept, efalizumab, infliximab, rituximab, and golimumab.

### Immunosuppressive Agents

The compositions described herein can also contain one or more immunosuppressive agents. Non-limiting examples of immunosuppressive agents include mycophenolate, ciclosporin, cyclosporine, tacrolimus, sirolimus, and pimecrolimus. Additional immunosuppressive agents are known in the art.

### Chemotherapeutic Agents

In some embodiments, the compositions further contain one or more chemotherapeutic agents. Non-limiting examples of chemotherapeutic agents include alkylating agents (e.g., cyclophosphamide, mechlorethamine, chlorambucil, and melphalan), anthracyclines (e.g., daunorubicin, doxorubicin, epirubicin, idarubicin, mitoxantrone, and valrubicin), taxanes (e.g., paxlitaxel and docetaxel), epothilones, histone deacetylase inhibitors (e.g., vorinostat and romidepsin), topoisomerase II inhibitors (e.g., etoposide, teniposide, and tafluposide), kinase inhibitors (e.g., bortezomib, erlotinib, gefitinib, imatinib, and vismodegib), bevacizumab, cetuximab, ipilimumab, ipilimumab, ofatumumab, ocrelizumab, panitumab, rituximab, vemurafenib, herceptin, nucleotide analogs (e.g., azacitidine, azathioprine, capecitabine, cytarabine, doxifluridine, fluorouracil, gemcitabine, hydroxyurea, mercaptopurine, methotrexate, and thioguanine), peptide antibiotics (e.g., bleomycin and actinomycin), platinum-based agents (e.g., carboplatin, cisplatin, and oxaliplatin), retinoids (e.g., tretinoin, alitretinoin, and bexarotene), and vinca alkaloids (e.g., vinblastine, vincristine, vindesine, and vinorelbine).

### Analgesics

In some embodiments, the composition can further contain one or more analgesics. Any of the exemplary analgesics described herein or known in the art can be included in the compositions described herein. Non-limiting examples of analgesics include opioid drugs (e.g., morphine, opium, codeine, oxycodone, hydrocodone, diamorphine, dihydromorphine, pethidine, buprenorphine, fentanyl, methadone, meperidine, pentazocine, dipipanone, and tramadol), acetaminophen, venlafaxine, flupirtine, nefopam, gabapentin, pregabalin, orphenadrine, cyclobenzaprine, trazodone, clonidine, duloxetine and amitriptyline.

### Formulations and Dosages

Any of the compositions described herein can be a pharmaceutical composition. For example, a pharmaceutical composition containing a LILRB1 agonist, LILRB2 agonist, LILRB3 agonist, LILRB4 agonist, and/or LILRB5 agonist and, optionally, one or more additional agents selected from the group consisting of a myeloid-derived suppressor cell, a mobilizing agent, a JNK inhibitor, an anti-inflammatory agent, and an immunosuppressive agent can further contain one or more of a pharmaceutically acceptable excipient or buffer, an antimicrobial or antifungal agent, or a stabilizing protein (e.g., human serum albumin). In some embodiments, a pharmaceutical composition containing one or more of: an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5, LAIR1, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1, or a soluble LAIR2 protein, and/or an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2; and, optionally, one or both of a chemotherapeutic agent and an analgesic can further contain one or more of a pharmaceutically acceptable excipient or buffer, an antimicrobial or antifungal agent, or a stabilizing protein (e.g., human serum albumin)

Any of the compositions described herein can be formulated as a liquid for systemic administration. In some embodiments, the compositions are formulated for intraarterial, epidural, intraluminal, intravenous, intraperitoneal, intrathecal, ocular, nasal, intramuscular, intraductal, rectal, or subcutaneous administration.

In some embodiments, the compositions are formulated as a solid. In some embodiments, the compositions are formulated for oral or topical (e.g., transdermal) administration. In some embodiments, the compositions are formulated as a suppository.

In some embodiments, the compositions are encapsulated in nanomaterials for targeted delivery (e.g., encapsulated in a nanomaterial having one or more tissue- or cell-targeting molecules on its surface). In some embodiments, the compositions are formulated as an emulsion or as a liposome-containing composition. In some embodiments, the compositions are formulated for sustained release (e.g., formulated in a biodegradable polymers or in nanoparticles). In some embodiments, the compositions are formulated in an implantable device that allows for sustained release of one or more of a LILRB1 agonist, a LILRB2 agonist, a LILRB3 agonist, a LILRB4 agonist, or a LILRB5 agonist; an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LA1R2 mRNA in a mammalian cell; a soluble L1LRB1 protein, a soluble LILRB2 protein, a soluble LILRB3 protein, a soluble LILRB4 protein, a soluble LILRB5 protein, a soluble LAIR1 protein, a soluble LAIR2 protein; an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2; a MDSC; a mobilizing agent; a JNK inhibitor; an anti-inflammatory agent; an immunosuppressive agent; a chemotherapeutic agent; and/or an analgesic.

Pharmaceutical compositions are formulated to be compatible with their intended route of administration or the intended target tissue, e.g., systemic or local administration. In some embodiments, the composition is delivered to an inflamed tissue in the mammal (e.g., by intramuscular, subcutaneous, intraperitoneal, intraarticular, or intrathecal injection). In some embodiments, the composition is delivered proximal to a tumor or a site of inflammation in a mammal. In some embodiments, the compositions are formulated for oral, intravenous, intradermal, subcutaneous, transmucosal (e.g., nasal sprays are formulated for inhalation), or transdermal (e.g., topical ointments, salves, gels, patches, or creams as generally known in the art) administration. The compositions can include a sterile diluent (e.g., sterile water or saline), a fixed oil, polyethylene glycol, glycerine, propylene glycol, or other synthetic solvents; antibacterial or antifungal agents, such as benzyl alcohol or methyl parabens, chlorobutanol, phenol, ascorbic acid, thimerosal, and the like; antioxidants, such as ascorbic acid or sodium bisulfite; chelating agents, such as ethylenediaminetetraacetic acid; buffers such as acetates, citrates, or phosphates; and isotonic agents, such as sugars (e.g., dextrose), polyalcohols (e.g., manitol or sorbitol), or salts (e.g., sodium chloride). Liposomal suspensions can also be used as pharmaceutically acceptable carriers (see, e.g., U.S. Pat. No. 4,522,811; herein incorporated by reference). Preparations of the compositions can be formulated and enclosed in ampules, disposable syringes, or multiple dose vials that prevent exposure of the caged tamoxifen or caged tamoxifen derivative molecules to light. Where required (as in, for example, injectable formulations), proper fluidity can be maintained by, for example, the use of a coating such as lecithin, or a surfactant. Absorption of one or more of a LILRB1 agonist, a LILRB2 agonist, a LILRB3 agonist, a LILRB4 agonist, or a LILRB5 agonist; an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LTLRB1, LILRB2, LILRB3, L1LRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, or a soluble LAIR2 protein; an agent thats inhibit signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2; a MDSC; a mobilizing agent; a JNK inhibitor; an anti-inflammatory agent; an immunosuppressive agent; a chemotherapeutic agent; and an analgesic can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the agents can be included in pills, capsules, troches and the like, and can contain any of the following ingredients, or compounds of a similar nature: a binder, such as microcrystalline cellulose, gum tragacanth, or gelatin; an excipient, such as starch or lactose; a disintegrating agent, such as alginic acid, Primogel, or corn starch; a lubricant, such as magnesium stearate; a glidant, such as colloidal silicon dioxide; a sweetening agent, such as sucrose or saccharin; or a flavoring agent, such as peppermint, methyl salicylate, or orange flavoring.

The compositions described herein can be formulated for ocular or parenteral (e.g., oral) administration in dosage unit form (i.e., physically discrete units containing a predetermined quantity of active compound for ease of administration and uniformity of dosage). Toxicity and therapeutic efficacy of compositions can be determined by standard pharmaceutical procedures in cell cultures or experimental animals. One can, for example, determine the LD50 (the dose lethal to 50% of the population) and the ED50 (the dose therapeutically effective in 50% of the population), the therapeutic index being the ratio of LD50:ED50. Compositions that exhibit high therapeutic indices are preferred. Where a composition exhibits an undesirable side effect, care should be taken to target the composition to the site of the affected or targeted tissue (the aim being to minimize potential damage to unaffected cells and, thereby, reduce side effects). Toxicity and therapeutic efficacy can be determined by other standard pharmaceutical procedures.

In some embodiments, the compositions described herein are formulated in a single dosage form. In some embodiments, a single dosage of the composition contains between 1 mg to 500 mg, between 1 mg and 400 mg, between 1 mg and 300 mg, between 1 mg and 250 mg, between 1 mg and 200 mg, between 1 mg and 100 mg, and between 1 mg and 50 mg of each of one or more of a LILRB1 agonist, a LILRB2 agonist, a LILRB3 agonist, a LILRB4 agonist, and a LILRB5 agonist; an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, or a soluble LAIR2 protein; an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2; a MDSC; a mobilizing agent; a JNK inhibitor; an anti-inflammatory agent; an immunosuppressive agent; a chemotherapeutic agent; and an analgesic. In some embodiments, the compositions contain at least 1×10⁷, at least 1×10⁸, at least 1×10⁹, and 1×10¹⁰ MDSCs.

Also provided herein are kits that contain at least one dose of any of the compositions described herein. In some embodiments, the kits can further include an item for use in administering a composition (e.g., any of the compositions described herein) to the mammal (e.g., a syringe, e.g., a pre-filled syringe). In some embodiments, the kits contain one or more doses (e.g., at least two, three, four, five, six, seven, eight, nine, ten, eleven, twelve, thirteen, fourteen, twenty, thirty, or fourty doses) (e.g., oral doses) of any of the compositions described herein. In some embodiments, the kit further contains instructions for administering the composition (or a dose of the composition) to a mammal (e.g., a mammal having pain).

In some embodiments, the kits contain a composition containing at least one LILRB1 agonist, LILRB2 agonist, LILRB3 agonist, LILRB4 agonist, and/or LILRB5 agonist (e.g., any of the LILRB1, LILRB2, LILRB3, LILRB4, and/or LILRB5 agonists described herein), and a composition containing one or more of a MDSC, mobilizing agent, JNK inhibitor, anti-inflammatory agent, and immunosuppressive agent (e.g., any of the MDSCs, mobilizing agents, JNK inhibitors, anti-inflammatory agents, and/or analgesics described herein). In some embodiments, the kits contain a composition containing a one or more of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, or a soluble LAIR2 protein, and/or an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2; and a composition containing a chemotherapeutic agent and/or an analgesic. In some embodiments, the kit further contains instructions for performing any of the methods described herein.

### Methods for Decreasing Pro-Inflammatory Immune Response or Treating an Autoimmune Disease, Inflammation, or Transplant Rejection

Also provided are methods of decreasing a pro-inflammatory immune response in a mammal that include administering to the mammal a therapeutically effective amount of a LILRB1 agonist, LILRB2 agonist, LILRB3 agonist, LILRB4 agonist, and/or LILRB5 agonist (e.g., any of the LILRB1, LILRB2, LILRB3, LILRB4, and/or LILRB5 agonists described herein). In some embodiments of any of the methods of decreasing a pro-inflammatory immune response described herein, the LILRB1, LILRB2, LILRB3, LILRB4, and/or LILRB5 agonist is not glatiramer acetate. Also provided are methods of treating inflammation, an autoimmune disease, or transplant rejection in a mammal that include administering to the mammal a therapeutically effective amount of a LILRB1 agonist, LILRB2 agonist, LILRB3 agonist, LILRB4 agonist, and/or LILRB5 agonist (e.g., any of the LILRB1, LILRB2, LILRB3, LILRB4, and/or LILRB5 agonists described herein). In some embodiments of any of the methods of treating inflammation, autoimmune disease, or transplant rejection described herein, the LILRB1, LILRB2, LILRB3, LILRB4, and/or LILRB5 agonist is not glatiramer acetate.

In some embodiments, the LILRB1, LILRB2, LILRB3, LILRB4, or LILRB5 agonist is glatiramer acetate. In some embodiments, the LILRB1, LILRB2, LILRB3, LILRB4, or LILRB5 agonist is an Angptl-1, Angptl-2, Angptl-3, Angptl-4, Angptl-5, Angptl-6, or Angptl-7 protein. In some embodiments, the Angptl-1, Angptl-2, Angptl-3, Angptl-4, Angptl-5, Angptl-6, or Angptl-7 protein is an endogenous mammalian Angptl-1, Angptl-2, Angptl-3, Angptl-4, Angptl-5, Angptl-6, or Angplt-7 protein (e.g., a human Angptl-1, Angptl-2, Angptl-3, Angptl-4, Angptl-5, Angptl-6, or Angptl-7 protein, e.g., SEQ ID NO: 1, 3, 5, 7, 43, 45, or 47). In some embodiments, the Angptl-1, Angptl-2, Angptl-3, Angptl-4, Angptl-5, Angptl-6, or Angptl-7 protein is at least 80% identical (e.g., at least 85%, 90%, 91%, 92%, 93%, 94%, 95%, 96%, 97%, 98%, 99%, or 100% identical) to an endogenous Angptl-1, Angptl-2, Angptl-3, Angptl-4, Angptl-5, Angptl-6, or Angptl-7 protein (e.g., SEQ ID NO: 1, 3, 5, 7, 43, 45, or 47) and has the ability to bind to and activate LILRB1, LILRB2, LILRB3, LILRB4, and/or LILRB5 signaling.

In some embodiments, the mammal (e.g., human) has been identified or suspected of having an elevated pro-inflammatory immune response. An elevated pro-inflammatory immune response can be detected in the mammal by the observation of one or more symptoms of inflammation described herein, an elevated level of one or more pro-inflammatory markers described herein (e.g., an elevation in one or more of C-reactive protein, 1L-1α, IL-1β, TNF-α, IL-6, IL-8, IL-23, IL-17, and matrix metalloproteases in a mammal compared to the corresponding levels of these proteins in a healthy mammal), and/or an increase in effector T-cells. A decrease in a pro-inflammatory immune response in a mammal can be detected by a decrease in the number of symptoms of inflammation in the mammal (e.g., any of the symptoms of inflammation described herein) and/or a decrease in the frequency and/or severity of one or more symptoms of inflammation in the mammal. A decrease in a pro-inflammatory immune response in a mammal can be detected by a decrease in one or more pro-inflammatory proteins in a mammal (e.g., a decrease following treatment or at a later time point in treatment) (e.g., a decrease in the levels of one or more of C-reactive protein, IL-1α, IL-1β, TNF-α, IL-6, IL-8, IL-23, IL-17, and matrix metalloproteases). In some embodiments, a decrease in pro-inflammatory immune response in a mammal can be detected by a decrease in the population of effector T-cells, an increase in Treg activation, and/or T-cell suppression.

In some embodiments, the mammal (e.g., human) has been previously diagnosed as having inflammation (e.g., acute or chronic inflammation). A mammal having inflammation often has one or more of the following symptoms: pain, sensation of heat, redness of skin, swelling of tissue, and loss of function in an affected tissue. In some embodiments, the mammal may experience inflammation in a specific tissue of his or her body (i.e., localized inflammation). A mammal can be diagnosed as having inflammation based on the presentation of one or more of the symptoms of inflammation described herein and/or based on molecular diagnostic assays that measure an elevated level of one or more inflammatory proteins in the mammal (e.g., an elevation in one or more of C-reactive protein, IL-1α, IL-1β, TNF-α, 1L-6, IL-8, IL-23, IL-17, and matrix metalloproteases). An effective treatment of inflammation can be detected by a decrease in the number of symptoms of inflammation in a mammal or a reduction in the severity or frequency of one or more symptoms of inflammation in a mammal. An effective treatment of inflammation in a mammal can also be detected by a decrease in the level of one or more pro-inflammatory proteins (e.g., a decrease in one or more of C-reactive protein, IL-1α, IL-1β, TNF-α, IL-6, IL-8, IL-23, IL-17, and matrix metalloproteases) in a mammal (as compared to the levels of these proteins in the mammal prior to treatment or at an earlier time point in treatment, or a control level of these proteins in a healthy mammal). An effective treatment of inflammation in a mammal can also be detected by a decrease in the population of effector T-cells, an increase in Treg activation, and/or T-cell suppression.

In some embodiments, the mammal can be diagnosed or suspected of having an autoimmune disease. Non-limiting examples of autoimmune diseases include acute disseminated encephalomyelitis, alopecia areata, ankylosing spondylitis, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autimmune uveitis, Celiac disease, Chagas disease, cold agglutinin disease, Crohn's disease, Dercum's disease, dermatomyositis, diabetes mellitus type I, endometriosis, eosinophil fasciitis, gastrointestinal pemphigoid, glomerulonephritis, Goodpasture's syndrome, Graves' disease, Guillain-Barre syndrome, Hashimoto's encephalopathy, Hashimoto's thyroiditis, hidradenitis suppurativa, idiopathic thrombocytopenic purpura, interstitial cystitis, Kawasaki's disease, lupus erythematosus, mixed connective tissue disease, morphea, multiple sclerosis, myasthenia gravis, pemphigus vulgaris, pernicious anaemia, polymyositis, primary biliary cirrhosis, psoriasis, psoriatic arthritis, relapsing polychondritis, rheumatoid arthritis, Sjogren's syndrome, temporal arteritis, tranverse myelitis, ulcerative colitis, undifferentiated connective tissue disease, vasculitis, vitiligo, and Wegener's granulomosis. The symptoms of an autoimmune disease vary depending on the specific autoimmune disease in the mammal. Non-limiting examples of symptoms of autoimmune diseases include: fatigue, muscle and joint pain, muscle weakness, swollen glands, inflammation, susceptibility to infections, sleep disturbances, weight loss or gain, low blood sugar (except for diabetes mellitus type I), blood pressure changes, Candida yeast infections, allergies, digestive problems, anxiety, depression, memory problems, thyroid problems, recurrent headaches, low grade fever, and recurrent miscarriage (for women). Symptoms of autoimmune disease can also include the loss of function of a particular tissue in the mammal that is targeted by the immune system (e.g., loss of pancreatic p-cell function in mammals with diabetes mellitus type I, and a loss of thyroid hormone production in mammals with Hashimoto's thyroiditis). A mammal can be diagnosed as having an autoimmune disease by the observation of one or more symptoms of an autoimmune disease in the mammal (e.g., any of the symptoms of an autoimmune disease described herein). An effective treatment of an autoimmune disorder can be detected by a decrease in the number of symptoms of an autoimmune disorder in a mammal, and/or by a decrease in the severity or frequency of one or more symptoms of an autoimmune disorder in a mammal. An effective treatment of an autoimmune disorder can also be detected by a decrease in inflammation observed in the mammal (e.g., using any of the methods described herein or known in the art).

In some embodiments, the mammal is diagnosed as having transplant rejection or is suspected of having transplant rejection. In some embodiments, the mammal has been selected for transplantation of tissue (e.g., an allograft or a xenograft) and has not yet received the transplant. In some embodiments, the transplanted tissue is kidney, lung, heart, skin, liver, bone marrow, or cornea. Tissue rejection can be detected in a mammal by an increase in inflammation in the mammal (e.g., using any of the methods described herein or known in the art). A mammal having tissue rejection often presents with one or more of the following symptoms: pain at the site of the transplant, flu-like symptoms, fever, weight changes, swelling, changes in the heart rate, and urinating less often. Effective treatment of tissue rejection in a mammal can be observed by a decrease in the number of symptoms of transplant rejection in the mammal and/or a decrease in the severity or frequency of one or more symptoms of transplant rejection in a mammal. Effective treatment of tissue rejection in a mammal can also be observed by a decrease in inflammation in the mammal (e.g., by detecting any of the symptoms of inflammation or any of the pro-inflammatory proteins described herein).

The mammal may be female or male, and may be an adult or juvenile (e.g., an infant). The mammal may have been previously treated with anti-inflammatory agent and/or immunosuppressive agent and/or responded poorly to the prior anti-inflammatory agent and/or immunosuppressive agent. Where the mammal is an adult, the mammal may be, e.g., between 18 to 20 years old or at least or about 20, 25, 30, 35, 40, 45, 50, 55, 60, 65, 70, 75, 80, 85, 90, 95, or at least or about 100 years old.

The LIlRB1, LILRB2, LILRB3, LILRB4, and/or LILRB5 agonist may be administered by intravenous, intraarterial, subcutaneous, intraperitoneal, intramuscular, ocular, intraarticular, or intrathecal administration. In some instances, the LILRB1, LILRB2, LILRB3, LILRB4, and/or LILRB5 agonist is administered by local administration to an inflamed tissue in the mammal. In other instances, the LILRB1, LILRB2, LILRB3, LILRB4, and/or LILRB5 agonist is systemically delivered to the mammal. Combinations of such treatments are contemplated by the present invention.

The LILRB1, LILRB2, LILRB3, LILRB4, and/or LILRB5 agonist can be administered by a medical professional (e.g., a physician, a physician's assistant, a nurse, a nurse's assistant, or a laboratory technician) or veterinary professional. Alternatively or in addition, the LILRB1, LILRB2, LILRB3, LILRB4, and/or LILRB5 agonist can be self-administered by a human, e.g., the patient her/himself. The LILRB1, LILRB2, LILRB3, LILRB4, and/or LILRB5 agonist can be administered in a hospital, a clinic, or a primary care facility (e.g., a nursing home), or any combination thereof.

The appropriate amount (dosage) of the LILRB1, LILRB2, LILRB3, LILRB4, and/or LILRB5 agonist administered can be determined by a medical professional or a veterinary professional based on a number of factors including, but not limited to, the type of inflammation (e.g., acute or chronic inflammation), the specific autoimmune disease, the specific transplated tissue being rejected, the route of administration, the severity of inflammation or transplant rejection, the mammal's responsiveness to other treatments, the health of the mammal, the mammal's mass, the other therapies administered to the mammal, the age of the mammal, the sex of the mammal, and any other co-morbidity present in the mammal.

A medical professional or veterinary professional having ordinary skill in the art can readily determine the effective amount of the LILRB1, LILRB2, LILRB3, LILRB4, and/or LILRB5 agonist that is required. For example, a physician or veterinarian could start with doses of the LILRB1, LILRB2, LILRB3, LILRB4, and/or LILRB5 agonist (e.g., any of the LILRB1, LILRB2, LILRB3, LILRB4, and/or LILRB5 agonists described herein) at levels lower than that required to achieve the desired therapeutic effect and then gradually increase the dose until the desired effect is achieved.

In some embodiments, the mammal is administered a dose of between 1 mg to 500 mg of any of the LILRB1, LILRB2, LILRB3, LILRB4 and/or LILRB5 agonists described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg).

In some embodiments, the mammal is further administered a MDSC (e.g., any of the MDSC subsets described herein), a mobilizing agent (e.g., any of the mobilizing agents described herein), a JNK inhibitor (e.g., any of the JNK inhibitors described herein), an anti-inflammatory agent (e.g., any of the anti-inflammatory agents described herein), and/or an immunosuppressive agent (e.g., any of the immunosuppressive agents described herein). In some embodiments, the mammal is administered a dose of between 1 mg to 500 mg each of any of the MDSCs, mobilizing agents, JNK inhibitors, anti-inflammatory agents, and/or immunosuppressive agents described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg of each). The one or more of a MDSC, mobilizing agent, JNK inhibitor, anti-inflammatory agent, and immunosuppressive agent can be administered to the mammal at substantially the same time as the LILRB1, LILRB2, LILRB3, LILRB4, and/or LILRB5 agonist. Alternatively or in addition, the one or more of a MDSC, mobilizing agent, JNK inhibitor, anti-inflammatory agent, and immunosuppressive agent may be administered to the mammal at one or more time points other than the time point at which the LILRB2 and/or LILRB4 agonist is administered. In some embodiments, the one or more of a MDSC, mobilizing agent, JNK inhibitor, anti-inflammatory agent, and immunosuppressive agent is formulated together with an LILRB1, LILRB2, LILRB3, LILRB4, and/or LILRB5 agonist (e.g., using any of the exemplary formulations and compositions described herein). In some embodiments, the one or more of a MDSC, mobilizing agent, JNK inhibitor, anti-inflammatory agent, and immunosuppressive agent are formulated in a first dosage form, and the LILRB1, LILRB2, LILRB3, LILRB4, and/or LILRB5 agonist is formulated in a second dosage form. In some embodiments where the one or more of a MDSC, mobilizing agent, JNK inhibitor, anti-inflammatory agent, and immunosuppressive agent are formulated in a first dosage form, and the LILRB1, LILRB2, LILRB3, LILRB4, and/or LILRB5 agonist is formulated in a second dosage form, the first dosage form and the second dosage form can be formulated for the same route of administration (e.g., oral, subcutaneous, intramuscular, intravenous, intaarterial, intrathecal, and intraperitoneal administration) or can be formulated for different routes of administration (e.g., the first dosage form formulated for oral administration and the second dosage form formulated for subcutaneous administration). Combinations of such treatment regimes are clearly contemplated in the present invention.

The amount of the LILRB1, LILRB2, LILRB3, LILRB4, and/or LILRB5 agonist (and optionally, one or more of a MDSC, a mobilizing agent, a JNK inhibitor, an anti-inflammatory agent, and an immunosuppressive agent) administered will depend on whether the administration is local or systemic. In some embodiments, the mammal is administered more than one dose of the LILRB1, LILRB2, LILRB3, LILRB4, and/or LILRB5 agonist. In some embodiments, the mammal is administered more than one dose of any of the compositions described herein. In some embodiments, the mammal is administered a dose of a LILRB1, LILRB2, LILRB3, LILRB4, and/or LILRB5 agonist at least once a month (e.g., at least twice a month, at least three times a month, at least four times a month, at least once a week, at least twice a week, three times a week, once a day, or twice a day).

In some embodiments, a LILRB1, LILRB2, LILRB3, LILRB4, and/or LILRB5 agonist is administered to a mammal chronically. In some embodiments, any of the compositions described herein is administered to the mammal chronically. Chronic treatments include any form of repeated administration for an extended period of time, such as repeated administrations for one or more months, between a month and a year, one or more years, or longer. In some embodiments, chronic treatments can involve regular administrations, for example one or more times a day, one or more times a week, or one or more times a month. In general, a suitable dose such as a daily dose of the LILRB1, LILRB2, LILRB3, LILRB4, and/or LILRB5 agonist will be the amount of the LILRB1, LILRB2, LILRB3, LILRB4, and/or LILRB5 agonist that is the lowest dose effective to produce a desired therapeutic effect. Such an effective dose will generally depend upon the factors described herein. If desired, the effective daily dose of the LILRB1, LILRB2, LILRB3, LILRB4, and/or LILRB5 agonist can be administered as two, three, four, five, or six or more sub-doses administered separately at appropriate intervals throughout the day, optionally, in unit dosage forms.

In some embodiments, the LILRB1, LILRB2, LILRB3, LILRB4, and/or LILRB5 agonist is formulated for sustained-release (e.g., formulated in a biodegradable polymer or a nanoparticle). In some embodiments, the LILRB1, LILRB2, LILRB3, LILRB4, and/or LILRB5 agonist is administered locally to the site of inflammation in a mammal, the site of transplanted tissue in a mammal, or a tissue affected by an autoimmune disease in a mammal. In some embodiments, the LILRB1, LILRB2, LILRB3, LILRB4, and/or LILRB5 agonist is administered systemically (e.g., oral, intravenous, intaarterial, intraperitoneal, intramuscular, or subcutaneous administration). In some embodiments, the LILRB1, LILRB2, LILRB3, LILRB4, and/or LILRB5 agonist is formulated for oral, intraglandular, periglandular, subcutaneous, interductal, intramuscular, intraperitoneal, intraarticular, rectal, epidural, intraarterial, transdermal, or intravenous administration.

### Methods for Increasing Pro-Inflammatory Immune Response and Treating Cancer or Infectious Disease in a Mammal

Also provided herein are methods of stimulating a pro-inflammatory immune response in a mammal that include administering to a mammal a therapeutically effective amount of at least one of: an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, or a soluble LAIR2 protein; and/or an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2. Also provided are methods of treating cancer in a mammal that include administering to the mammal a therapeutically effective amount of at least one of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, or a soluble LAIR2 protein; and/or an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2.

In some embodiments, an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LATR1 protein, or LAIR2 protein (e.g., an antibody or an antigen-binding antibody fragment that binds to Angptl-1, Angptl-2, Angptl-3, Angptl-4, Angplt-5, Angptl-6, Angptl-7, LILRB1, LILRB2, LILRB3, LILRB4, LILRB5, LAIR1, or LAIR2 or an aptamer) is administered to the mammal (e.g., any of the agents that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5, LAIR1, or LAIR2 protein described herein). In some embodiments, an antibody or antigen-binding antibody fragment that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is administered to the mammal. In some embodiments, the antibody that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is an IgG or an IgM antibody. In some embodiments, the antibody that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is a human or a humanized antibody. In some embodiments, the antigen-binding antibody fragment that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is a Fab fragment, a F(ab′)₂ fragment, a scFv fragment, or any of the other antigen-binding antibody fragments described herein. In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein is an aptamer (e.g., a nucleic acid or peptide aptamer).

In some embodiments, the the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell is an inhibitory RNA (e.g., siRNA), an antisense oligonucleotide, or a ribozyme (e.g., any of the oligonucleotides that decrease the expression of Angptl-1, Angptl-2, Angptl-3, Angptl-4, Angptl-5, Angptl-6, Angptl-7, LILRB1, LILRB2, LILRB3, LILRB4, LILRB5, LATR1, or LAIR2 mRNA in a mammalian cell described herein).

In some embodiments, the soluble LILRB2 protein contains a sequence that is at least 80% identical (e.g., at least 85%, 90%, 91%, 92%, 93%, 94%, 95%, 96%, 97%, 98%, 99%, or 100% identical) to the extracellular domain of an endogenous human LILRB2 protein (e.g., amino acids 1-418 of SEQ ID NO: 9). In some embodiments, the soluble LILRB4 protein contains a sequence that is at least 80% identical (e.g., at least 85%, 90%, 91%, 92%, 93%, 94%, 95%, 96%, 97%, 98%, 99%, or 100% identical) to the extracellular domain of an endogenous human LILRB4 protein (e.g., the extracellular domain of SEQ ID NO: 49). In some embodiments, the solube LILRB1 protein contains a sequence that is at least 80% identical (e.g., at least 85%, 90%, 91%, 92%, 93%, 94%, 95%, 96%, 97%, 98%, 99%, or 100% identical) to the extracellular domain of an endogenous human LILRB1 protein (e.g., the exemplary extracellular domain of human L1LRB1 protein described herein). In some embodiments, the solube LILRB3 protein contains a sequence that is at least 80% identical (e.g., at least 85%, 90%, 91%, 92%, 93%, 94%, 95%, 96%, 97%, 98%, 99%, or 100% identical) to the extracellular domain of an endogenous human LILRB3 protein (e.g., the exemplary extracellular domain of human LILRB3 protein described herein). In some embodiments, the solube LILRB5 protein contains a sequence that is at least 80% identical (e.g., at least 85%, 90%, 91%, 92%, 93%, 94%, 95%, 96%, 97%, 98%, 99%, or 100% identical) to the extracellular domain of an endogenous human LILRB5 protein (e.g., the exemplary extracellular domain of human LILRB5 protein described herein).

In some embodiments, an increase in pro-inflammatory immune response in a mammal can be detected as an increase in the levels of one or more pro-inflammatory proteins in the mammal (e.g., an increase in one or more of C-reactive protein, IL-1α, IL-1β, TNF-α, IL-6, IL-8, IL-23, IL-17, and matrix metalloproteases) or an increase in the number of effector T-cells (Teff) in the mammal (e.g., as compared to the levels of the one or more pro-inflammatory proteins in the mammal and/or the levels of effector T-cells in the mammal prior to treatment or compared to the levels of the one or more pro-inflammatory proteins and/or the levels of effector T-cells present in a control, healthy mammal)

In some embodiments, the mammal (e.g., human) has been previously diagnosed as having a cancer (e.g., any of the different types of cancer described herein). Non-limiting examples of cancer include: bladder cancer, breast cancer, colon cancer, colorectal cancer, endometrial caner, kidney cancer, lung cancer, melanoma, pancreatic cancer, prostate cancer, thyroid cancer, bile duct cancer, bone cancer, brain cancer, cervical cancer, cardiac tumors, esophageal cancer, eye cancer, gallbladder cancer, gastric cancer, head and neck cancer, heart cancer, liver cancer, laryngeal cancer, leukemia, lip and oral cavity cancer, lymphoma, melanoma, mesothelioma, mouth cancer, nasal cavity and paranasal sinus cancer, nasopharyngeal cancer, non-Hodgkin lymphoma, ovarian cancer, penile cancer, pituitary tumor, retinoblastoma, sarcoma, skin cancer, testicular cancer, throat cancer, thyroid cancer, urethral cancer, uterine cancer, vaginal cancer, and vulvar cancer. A mammal having cancer can present with one or more of the following symptoms: fatigue, lump or thickening that can be felt under the skin, weight changes, skin changes (e.g., yellowing, darkening or redness of the skin, sores that won't heal, or changes in existing moles), changes in bowel or bladder habits, persistent cough, difficulty swallowing, hoarseness, persistent indigestion or discomfort after eating, persistent, unexplained muscle or joint pain, and unexplained and persistent fevers or night sweats. The particular symptoms experienced by a mammal will depend on the particular type of cancer. A mammal can be diagnosed as having a cancer based on the observation of one or more symptoms of cancer in the mammal (e.g., any of the symptoms of cancer described herein or known in the art). A mammal can also be diagnosed as having a cancer based on imaging (e.g., magnetic resonance imaging, computed tomography, and/or X-ray) and/or tissue biopsy results. A mammal can also be diagnosed as having a cancer based using molecular diagnostic tests (e.g., based on the detection of prostate specific antigen, or mutations in breast cancer susceptibility 2 protein, breast cancer susceptibility 1 protein, or a tumor suppressor protein (e.g., p53)). Additional methods for diagnosing a mammal as having cancer are known in the art. Efficacy of treatment of a cancer can be detected by a decrease the number of symptoms of a cancer in a mammal (e.g., any of the symptoms of cancer described herein or known in the art) and/or a decrease in the frequency and/or severity of one or more symptoms of cancer in a mammal (e.g., any of the symptoms described herein or known in the art). An effective treatment of cancer in a mammal can also be assessed by a decrease in the rate of growth of a tumor in a mammal (e.g., compared to the rate of tumor growth in the mammal prior to administration of treatment or compared to a control mammal having the same type of cancer not administered a treatment or administered a different treatment). An effective treatment of cancer in a mammal can also be observed by an increase in the length of remission of cancer in the mammal (e.g., compared to a control mammal having the same type of cancer not administered a treatment or administered a different treatment).

The mammal may be female or male, and may be an adult or juvenile (e.g., an infant). The mammal may have been previously treated with a chemotherapeutic agent and/or analgesic and/or responded poorly to the chemotherapeutic agent and/or analgesic. The mammal may have non-metastatic cancer. In some embodiments, the mammal can have metastatic cancer. Where the mammal is an adult, the mammal may be, e.g., between 18 to 20 years old or at least or about 20, 25, 30, 35, 40, 45, 50, 55, 60, 65, 70, 75, 80, 85, 90, 95, or at least or about 100 years old.

The agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, may be administered by intravenous, intraarterial, subcutaneous, intraperitoneal, intramuscular, ocular, intraarticular, or intrathecal administration. In some instances, the agent that specifically binds to an endogenous Angptl-1 protein, Angptl-2 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, or the soluble LAIR2 protein; and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, is administered by local administration to a tumor in the mammal In other instances, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, is systemically delivered to the mammal Combinations of such treatments are contemplated by the present invention.

The agent that specifically binds to an endogenous Angptl -1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, can be administered by a medical professional (e.g., a physician, a physician's assistant, a nurse, a nurse's assistant, or a laboratory technician) or veterinary professional. Alternatively or in addition, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, can be self-administered by a human, e.g., the patient her/himself. The agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LATR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2, can be administered in a hospital, a clinic, or a primary care facility (e.g., a nursing home), or any combination thereof.

The appropriate amount (dosage) of the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 administered can be determined by a medical professional or a veterinary professional based on a number of factors including, but not limited to, the type of cancer, the route of administration, the stage of cancer (e.g., tumor burden), the mammal's responsiveness to other cancer treatments (e.g., chemotherapeutic agents), the health of the mammal, the mammal's mass, the other therapies administered to the mammal, the age of the mammal, the sex of the mammal, and any other co-morbidity present in the mammal.

A medical professional or veterinary professional having ordinary skill in the art can readily determine the effective amount of the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 that is required. For example, a physician or veterinarian could start with doses of the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, L1LRB1, LILRB2, LILRB3, LILRB4, LILRB5, LAIR1, or LAIR2 (e.g., any of the agents that specifically bind to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotides that decrease the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 proteins, the soluble LILRB2 proteins, the soluble LILRB3 proteins, the soluble LILRB4 proteins, the soluble LILRB5 proteins, the soluble LAIR1 proteins, the soluble LAIR2 proteins, and the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 described herein) at levels lower than that required to achieve the desired therapeutic effect and then gradually increase the dose until the desired effect is achieved.

In some embodiments, the mammal is administered a dose of between 1 mg to 500 mg each of one or more of any of the agents that specifically bind to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR protein, or LAIR2 protein; the oligonucleotides that decrease the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 proteins, the soluble LILRB2 proteins, the soluble LILRB3 proteins, the soluble LILRB4 proteins, the soluble LILRB5 proteins, the soluble LAIR1 proteins, the soluble LAIR2 proteins, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg of each).

In some embodiments, the mammal is further administered a chemotherapeutic agent (e.g., any of the chemotherapeutic agents described herein or known in the art) and/or an analgesic (e.g., any of the analgesics described herein or known in the art). In some embodiments, the mammal is administered a dose of between 1 mg to 500 mg each of one or more of any of the chemotherapeutic agents and/or analgesics described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, or between 5 mg and 40 mg each). The chemotherapeutic agent and/or the analgesic can be administered to the mammal at substantially the same time as the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LTLRB5 protein, the soluble LAIR1 protein, the soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2. Alternatively or in addition, the chemotherapeutic agent and/or the analgesic may be administered to the mammal one or more time points other than the time point at which the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LTLRB5, LATR1, or LAIR2 mRNA in a mammalian cell; the soluble LTLRB1 protein, the soluble LILRB2 protein, the soluble LILRB3 protein, the soluble LILRB4 protein, the soluble LILRB5 protein, the soluble LAIR1 protein, soluble LAIR2 protein, and/or the agent that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is administered. In some embodiments, the chemotherapeutic agent and/or the analgesic is formulated together with an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, soluble LILRB2 protein, soluble LILRB3 protein, soluble LILRB4 protein, a soluble LILRB5 protein, a soluble LAIR1 protein, a soluble LAIR2 protein, and/or an agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 (e.g., using any of the exemplary formulations and compositions described herein). In some embodiments, the chemotherapeutic agent and/or the analgesic are formulated in a first dosage form, and the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is formulated in a second dosage form. In some embodiments where the chemotherapeutic agent and/or the analgesic are formulated in a first dosage form, and the agent that specifically binds to an endogenous Angptl-1 protein, Angptl-2 protein, Angplt-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is formulated in a second dosage form, the first dosage form and the second dosage form can be formulated for the same route of administration (e.g., oral, subcutaneous, intramuscular, intravenous, intaarterial, intrathecal, and intraperitoneal administration) or can be formulated for different routes of administration (e.g., the first dosage form formulated for oral administration and the second dosage form formulated for subcutaneous administration). Combinations of such treatment regimes arc clearly contemplated in the present invention.

The amount of the agent that specifically binds to an endogenous Angptl-1 protein, Angptl-2 protein, Angplt-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angtp1-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 (and optionally, a chemotherapeutic agent and/or analgesic) administered will depend on whether the administration is local or systemic. In some embodiments, the mammal is administered more than one dose of the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2. In some embodiments, the mammal is administered more than one dose of any of the compositions described herein. In some embodiments, the mammal is administered a dose of an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, a soluble LILRB5 protein, a soluble LAIR1 protein, a soluble LAIR2 protein, and/or an agent(s) that inhibits signaling pathway(s) initiated by Angptl -1, Angptl-2, Angptl-3, Angptl-4, Angptl-5, Angptl-6, Angptl-7, LILRB1, LILRB2, LILRB3, LILRB4, LILRB5, LAIR1, or LAIR2 at least once a month (e.g., at least twice a month, at least three times a month, at least four times a month, at least once a week, at least twice a week, three times a week, once a day, or twice a day).

In some embodiments, an agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; an oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; a soluble LILRB1 protein, a soluble LILRB2 protein, a soluble LILRB3 protein, a soluble LILRB4 protein, soluble LILRB5 protein, soluble LAIR1 protein, soluble LAIR2 protein, and/or agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is administered to a mammal chronically. In some embodiments, any of the compositions described herein is administered to the mammal chronically. Chronic treatments include any form of repeated administration for an extended period of time, such as repeated administrations for one or more months, between a month and a year, one or more years, or longer. In some embodiments, chronic treatments can involve regular administrations, for example one or more times a day, one or more times a week, or one or more times a month. In general, a suitable dose such as a daily dose of the agent that specifically binds to an endogenous Angptl-1 protein, Angptl-2 protein, Angptl-3 protein, Angptl-4 protein, Angptl-5 protein, Angptl-6 protein, Angptl-7 protein, L1LRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 will be the amount of the agent and/or oligonucleotide that is the lowest dose effective to produce a desired therapeutic effect. Such an effective dose will generally depend upon the factors described herein. If desired, the effective daily dose of the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 can be administered as two, three, four, five, or six or more sub-doses administered separately at appropriate intervals throughout the day, optionally, in unit dosage forms.

In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is formulated for sustained-release (e.g., formulated in a biodegradable polymer or a nanoparticle). In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is administered locally to the site of a tumor in the mammal. In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibit signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is administered systemically (e.g., oral, intravenous, intaarterial, intraperitoneal, intramuscular, or subcutaneous administration). In some embodiments, the agent that specifically binds to an endogenous Angptl-1 protein, Angptl-2 protein, Angptl-3 protein, Angptl-4 protein, Angptl-5 protein, Angptl-6 protein, Angptl-7 protein, LILRB1 protein, LILRB2 protein, LILRB3 protein, LILRB4 protein, LILRB5 protein, LAIR1 protein, or LAIR2 protein; the oligonucleotide that decreases the expression of Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 mRNA in a mammalian cell; the soluble LILRB1 protein, the soluble LILRB2 protein, the soluble LILRB3 protein, the soluble LILRB4 protein, the soluble LILRB5 protein, the soluble LAIR1 protein, the soluble LAIR2 protein, and/or the agent(s) that inhibits signaling pathway(s) initiated by Angptl-1, Angptl-2, Angptl-3, Angptl-4, Angptl-5, Angptl-6, Angptl-7, LILRB1, LILRB2, LILRB3, LILRB4, LILRB5, LAIR1, or LAIR2 is formulated for oral, intraglandular, periglandular, subcutaneous, interductal, intramuscular, intraperitoneal, intraarticular, rectal, epidural, intraarterial, transdermal, or intravenous administration.

### Screening Methods

Also provided are methods of identifying a candidate agent for treating an autoimmune disease, inflammation, infectious disease, or transplant rejection in a mammal that include: (a) contacting a LILRB1 protein, a LILRB2 protein, a LILRB3 protein, a LILRB4 protein, a LILRB5 protein, or a paired immunoglobulin-like receptor B (PIRB) protein with a test agent, and determining the amount of binding of the test agent to the LILRB1 protein, LILRB2 protein, LILRB3 protein, LILRB4 protein, LILRB5 protein, or PIRB protein; (b) determining whether the test agent activates LILRB1 protein, LILRB2 protein, LILRB3 protein, LILRB4 protein, LILRB5 protein, or PIRB protein signaling in a cell (e.g., any of the LILRB1, LILRB2, LILRB3, LILRB4, LILRB5, or PIRB signaling pathways described in the Examples, e.g., increased CAMKII, CAMKIV, SHP-1, and/or SHP-2 phosphorylation; decreased TNF-α and/or increased TGF-β and IL-10 secretion); and (c) selecting a test agent that binds to LILRB1 protein, LILRB2 protein, LILRB3 protein, LILRB4 protein, LILRB5 protein, or PIRB protein, and activates LILRB1 protein, LILRB2 protein, LILRB3 protein, LILRB4 protein, LILRB5 protein, or PIRB protein, respectively, as a candidate agent for treating an autoimmune disease, inflammation, infectious disease, or transplant rejection in a mammal In some embodiments, the LILRB1, LILRB2, LILRB3, LILRB4, LILRB5, or PIRB protein in (a) is expressed on the surface of a cell (e.g., a transformed cell that contains a nucleic acid encoding LILRB1, LILRB2, LILRB3, LILRB4, LILRB5, or PIRB protein (e.g., a human LILRB1, LILRB2, LILRB3, LILRB4, or LILRB5 protein)).

Also provided are methods of identifying a candidate agent for treating an autoimmune disease, inflammation, infectious disease, or transplant rejection in a mammal that include: determining whether the test agent activates LILRB1 protein, LILRB2 protein, LILRB3 protein, LILRB4 protein, LILRB5 protein, or PIRB protein signaling in a cell (e.g., any of the LILRB1, LILRB2, LILRB3, LILRB4, or LILRB5 signaling pathways described in the Examples, e.g., increased CAMKII, CAMKIV, SHP-1, and/or SHP-2 phosphorylation;

decreased TNF-α and/or increased TGF-β and IL-10 secretion); and selecting a test agent activates LILRB1 protein, LILRB2 protein, LILRB3 protein, LILRB4 protein, LILRB5 protein, or PIRB protein, respectively, as a candidate agent for treating an autoimmune disease, inflammation, infectious disease, or transplant rejection in a mammal.

In some embodiments, the cell in is a human cell. In some embodiments, the cell in is a T-cell. In some embodiments, the cell is a MDSC.

Some embodiments further include determining the ability of the selected test agent to induce M2 polarization in a MDSC, and further selecting an agent that has the ability to induce M2 polarization in a MDSC as a candidate agent for treating inflammation, autoimmune disease, or transplant rejection in a mammal. Some embodiments further include determining the ability of the selected test agent to increase the activation of Treg cells in a mammal, and further selecting an agent that has the ability to increase the activation of Treg cells in a mammal as a candidate agent for treating inflammation, autoimmune disease, or transplant rejection in a mammal. Some embodiments further include determining the ability of the selected agent to decrease inflammation in an animal model of inflammation (see, e.g., the various animal models of inflammation described in Stevenson et al., In Vivo Models of Inflammation, 2^{nd} Edition, Birkhauser Basel, 2006), and further selecting an agent that decreases inflammation in an animal model of inflammation as a candidate agent for treating inflammation, autoimmune disease, or transplant rejection in a mammal. Some embodiments further include determining the ability of the agent to decrease the levels of one or more pro-inflammatory proteins in an animal model of inflammation (e.g., any of the pro-inflammatory proteins described herein), and further selecting an agent that decreases the levels of one or more pro-inflammatory proteins in an animal model of inflammation as a candidate agent for treating inflammation, autoimmune disease, or transplant rejection in a mammal. Some embodiments of these methods further include generating a pharmaceutical composition for treating inflammation, autoimmune disease, or transplant rejection that includes the candidate agent.

Also provided are methods of identifying a candidate agent useful for treating cancer that include contacting an angiopoietin-like (Angpt1)-1 protein, an Angptl-2 protein, Angptl-3 protein, an Angptl-4 protein, an Angptl-5 protein, Angptl-6 protein, or an Angptl-7 protein with a test agent; determining whether the test agent binds to the Angptl-1 protein, the Angptl-2 protein, the Angptl-3 protein, the Angptl-4 protein, the Angptl-5 protein, the Angptl-6 protein, or the Angptl-7 protein; and selecting a test agent that specifically binds to the Angptl-1 protein, the Angptl-2 protein, the Angptl-3 protein, the Angptl-4 protein, the Angptl-5 protein, the Angptl-6 protein, or the Angptl-7 protein as a candidate agent for treating a cancer.

In some embodiments, the binding of the test agent to the Angptl-1 protein, the Angptl-2 protein, the Angptl-3 protein, the Angptl-4 protein, the Angptl-5 protein, the Antptl-6 protein, or the Angptl-7 protein is determined using, e.g., BioCoRE® or competitive binding assays (e.g., assays that utilize an antibody that binds to Angptl-1 protein, Angptl-2 protein, Angptl-3 protein, Angptl-4 protein, Angptl-5 protein, Angptl-6 protein, or Angptl-7 protein).

Some embodiments further include testing the ability of the test agent to decrease or prevent the binding of an Angptl-1, Angptl-2, Angptl-3, Angptl-4, Angplt-5, Angptl-6, or Angptl-7 protein to LILRB1, LILRB2, LILRB3, LILRB4, LILRB5, and/or PIRB protein, and further selecting an agent that decreases or prevents the binding of an Angptl-1, Angptl-2, Angptl-3, Angptl-4, Angplt-5, Angptl-6, or Angptl-7 protein to LILRB1, LILRB2, LILRB3, LILRB4, LILRB5, and/or PIRB protein as a candidate agent for treating a cancer in a mammal. Some embodiments further include testing the ability of the test agent to increase the population of T effector (Teff) cells in a mammal, and further selecting an agent that increases the population of Teff cells in a mammal as a candidate agent for treating cancer in a mammal. Some embodiments further include testing the ability of the test agent to increase the level of one or more pro-inflammatory proteins in a mammal (e.g., any of the pro-inflammatory proteins described herein or known in the art), and further selecting an agent that increases the level of one or more pro-inflammatory proteins in the mammal as a candidate agent for treating cancer in a mammal. Some embodiments further include administering the test agent to an animal model of cancer (see, e.g., Teicher, Tumor Models in Cancer Research, Humana Press, 2011), and further selecting a test agent that demonstrates efficacy in the animal model of cancer (e.g., efficacy assessed using any of the methods described herein or known in the art) as a candidate agent for treating cancer in a mammal.

Some embodiments of these methods further include generating a pharmaceutical composition for treating a cancer that includes the candidate agent.

The invention is further described in the following example, which does not limit the scope of the invention described in the claims.

### EXAMPLES

### Example 1

### Glatiramer Acetate Promotes M2-MDSC Polarization through Binding and Activation of PIR-B (a Homolog of LILRB2)

Experiments were performed to determine the effect of glatiramer acetate (GA) on the paired immunoglobulin-like receptor-B (PIR-B)-modulated cytokine profile change and polarization of MDSCs.

### Materials and Methods

Reagents and mice. SP600125 was purchased from Sigma, GA was purchased from Teva Neuroscience, HA peptide (¹¹⁰SFERFEIFPKE¹²⁰; SEQ ID NO: 15) was purchased from Washington Biotec, anti-PIR-A/B (6C1) was purchased from BD Biosciences, and anti-PIR-B was purchased from R&D. GA-FITC was synthesized by conjugation of GA with fluorescein isothiocyanate. PHA, anti-Gr-1, anti-CD115, anti-F4/80, anti-CD11b, anti-CD4, anti-CD25, anti-Foxp3, and isotype-matched antibodies were purchased from eBioscience.

Suppression and T_{reg} induction by MDSC. MDSC suppression assays were performed as described previously (Pan et al., Blood 111:219-228, 2008). Briefly, splenocytes (1×10⁵) from HA-TCR mice were co-cultured with serial dilutions of irradiated MDSCs in the presence of HA peptides (5 μg/mL). T_{reg} induction assays were performed as previously described in Ma et al., Immunity 34:385-395, 2011. Splenocytes (4×10⁶) from HA-TCR mice were co-cultured with irradiated MDSC (1×10⁶) in the presence of HA peptides. The percentage of T_{reg} was assessed by flow cytometric analysis.

Preparation of splenocytes, monocytes, and MDSCs. Splenocytes were obtained from HA-TCR Tg mice. Monocytes were purified from naïve mice. CD115⁺ MDSCs were purified from bone marrow cells using MACS columns (Huang et al., Cancer Res. 66:1123-1131, 2006). Human CD14⁺CD33⁻HLA-DR^{Low} MDSCs were sorted from the peripheral blood obtained from cancer patients.

Cytokine detection by enzyme-linked immunosorbent assay (ELISA). Culture supernatants were collected for measurement of mouse or human cytokines (IL-6, IL-10, IL-23, IFN-γ, TNF-α, TGF-β1, and IL-17A) by ELISA.

Immunoprecipitation and Immunoblot. MDSCs were stimulated with GA for 2 hours, followed by treatment with vehicle, LPS, or IFN-γ for an additional 30 minutes. Total cell lysates were prepared for immunoblot. For immunoprecipitation, total cell lysates from CD115⁺ MDSCs were incubated with vehicle or GA-FITC, then with anti-FITC and protein G bead. The precipitates and total lysates were analyzed by immunoblot as previously described in Ma et al., Immunity 34:385-395, 2011.

Reverse transcription-polymerase chain reaction (RT-PCR). cDNA was generated from total RNA by reverse transcriptase, and used as a template to perform RT-PCR analysis with a primer set for GAPDH mRNA (5′-TGGAGATTGTTGCCATCAACG-3′ (SEQ ID NO: 16) and 5′-CAGTGGATGCAGGGATGATGTTCTG-3′ (SEQ ID NO: 17) or Foxp3 mRNA (5′-CAGCTGCCTACAGTGCCCCTAG-3′ (SEQ ID NO: 18) and 5′-CATTTGCCAGCAGTGGGTAG-3′ (SEQ ID NO: 19)). The resulting PCR products were thereafter analyzed.

### Results

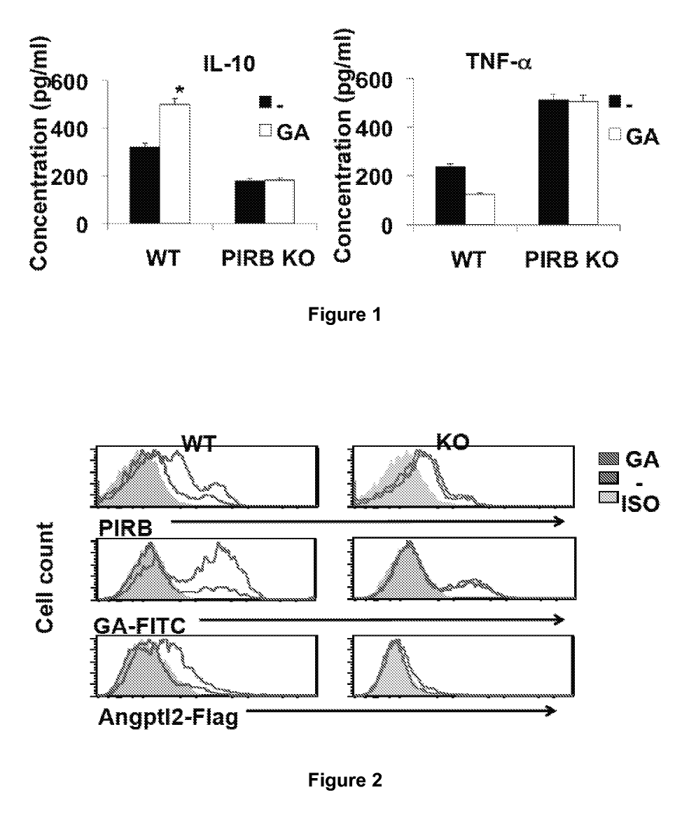

GA treatment led to increased production of IL-10 and reduced TNF-α in wild type MDSCs (FIG. 1). In contrast, GA treatment exerted no significant effect on cytokine production from PIR-B deficient (KO) MDSCs, indicating that PIR-B might be directly targeted by GA. Flow cytometry and immunoprecipitation were further used to detect direct interactions between GA and PIR-B expressed on the surface of MDSCs. The data show that FITC-conjugated GA specifically stained WT MDSCs in a pattern similar to staining with anti-PIR-B or Flag-tagged recombinant Angiopoetin-like 2 (Angptl-2). Unconjugated GA was able to block this interaction in WT MDSCs, but not PIR-B KO MDSCs (FIG. 2). The interaction of GA with PIR-B was further confirmed by immunoprecipitation. Anti-FITC magnetic beads and FITC-conjugated GA specifically pulled down PIR-B from lysates of WT, but not PIR-B KO, MDSCs. These data demonstrate that GA functions as a specific ligand for PIR-B on MDSCs.

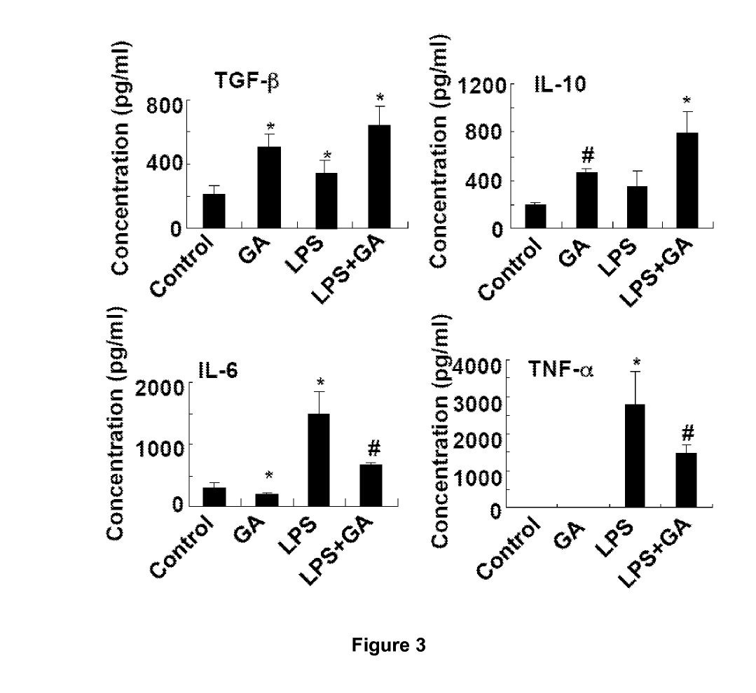

A further set of experiments were performed to examine the effects of GA on PIR-B signaling in the presence of LPS and IFN-γ. GA significantly inhibited the phosphorylation of NF-κB, STAT1, and p38 induced by IFN-γ or LPS. Significant reductions in IL-6 and TNF-α secretion, and increased production of IL-10 and TGF-β were observed when MDSCs were treated with GA followed by stimulation with LPS (FIG. 3). Taken together, these data indicate that GA acts on PIR-B directly to promote M2 polarization of MDSCs, and this M2 polarization enhances the production of suppressive cytokines by MDSCs.

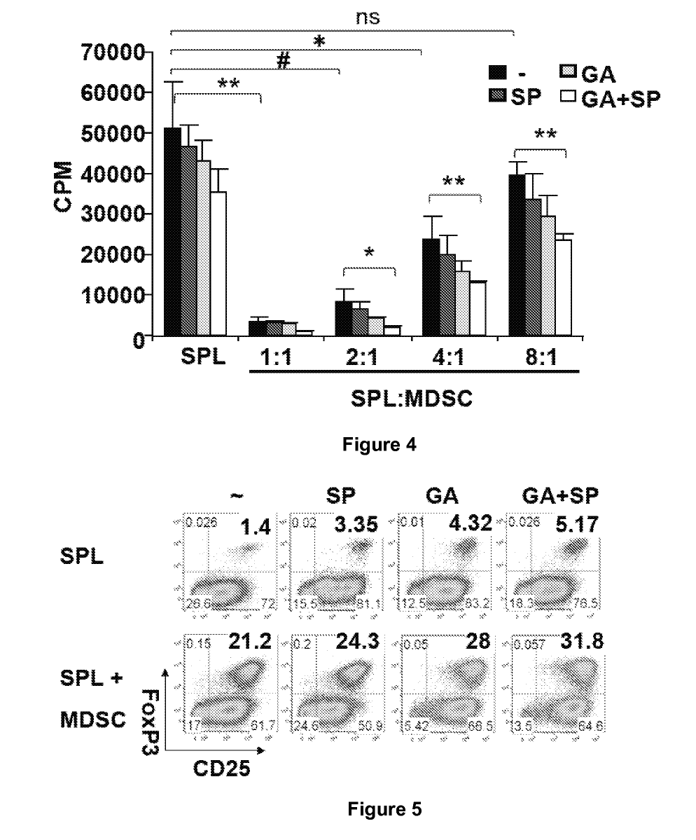

Multiple pathways, including inhibition of T-cell activation and induction of regulatory T-cells (T_{reg}), have been proposed to explain how M2 MDSCs suppress immune responses. An additional set of experiments were performed in order to study the effect of GA on PIR-B signaling and MDSC suppressive functions. As c-Jun N-terminal kinase (JNK) signaling plays a key role in inducing a pro-inflammatory immune response, the effect of a JNK inhibitor (SP600125) and/or GA on the immunoregulatory activities of MDSCs was examined. MDSC-mediated suppression of T-cell proliferation and T_{reg} activation were increased in cultures treated with GA or SP600125, and an even stronger effect was observed when both were used (FIGS. 4 and 5). The effect on T_{reg} activation correlated with elevated FoxP3 expression. A higher TGF-β and IL-10, and lower IL-6 and IL-23 production by MDSCs, and reduced IL-17 secretion by T-cells in cultures treated with GA or SP600125 was also observed. Profiling of maturation markers showed a higher percentage of cells expressing lower levels of MHC class II, CD80, CD86, CD11c, and F4/80 after treatment, indicating that spontaneous differentiation of MDSCs to mature cell types was blocked. The ability of MDSCs to activate T_{reg} was sustained over a longer period of time and was more pronounced in the presence of GA/SP600125 vs. MDSCs alone. In contrast, similarly treated monocytes did not exhibit T_{reg} activating activity. Hence, both GA and SP600125 can modulate MDSC differentiation and function, and their effect on MDSC-mediated T-cell suppression and T_{reg} activation was additive.

### Example 2

### Angiopoietin-Like Proteins Bind LILR Receptors on Human MDSCs and Induce MDSC Repopulation

A set of experiments were performed in order to identify the receptor(s) for antiopoietin-like proteins (Angptls).

### Materials and Methods

Mice. C57 BL/6 CD45.2 and CD45.1 mice, or NOD/SCID mice were purchased from the UT Southwestern Medical Center animal breeding core facility. The PirBTM mice (Sicken et al., Science 313:1795-1800, 2006) were obtained from MMRRC. The PirB knockout mice (Ujike et al., Nat. Immunol. 3:542-548, 2002) were from Tohoku University.

Plasmids and proteins. Plasmid CMV-Kozak-human Angl, Angptl-1, -2, -3, -4, -6, and -7 with FLAG tags at the C-termini were transfected into 293T cells using Lipofectamine 2000, and the conditioned medium at 48 hours was collected and different Angptl proteins were adjusted to the same level for flow cytometry-based binding experiments. Angptl-2-FLAG was purified using M2 resin. Purified GST-Angptl-5 was purchased from Abnova. Bacterially-expressed Flag-Angptl-2 and Angptl-2-Flag were constructed in pET-26b(+) vector, and GST-Angptls-FLAG in pGEX vector, and expressed and purified from bacteria. MSCV-LILRB2-IRES-GFP or control retrovirus infected BAF3 cells, or CMV-driven LILRAs, LILRBs, PirB, or LAIR1 transfected 293T cells harvested at 48 hours, or mononuclear human cord blood cells were incubated with Fc block and equal amounts of different FLAG-tagged Angptls at 4° C. for 60 minutes, followed by staining with anti-Flag-APC and propidium iodide. Anti-LILRB2-PE was used as indicated. The cells were analyzed using either a FACSCalibur or FACSAria instrument (Becton Dickinson).

Antibodies and shRNAs. Flow cytometry antibodies anti-CD34-FITC, anti-CD38-PE, anti-CD9O-PE/Cy5.5, biotinylated lineage cocktail, anti-Kit-APC, anti-Sca-1-FITC, anti-Mac-1-APC, anti-Gr-1-PE, anti-CD3-APC, and anti-B220-PE were purchased from BD Biosciences. The manufacturers and catalog numbers for other antibodies are as follows: anti-LILRB1, Biolegend (33707); anti-LILRB2, eBioscience (12-5149); anti-LILRB3, eBioscience (12-5159); anti-LILRB4, eBiosciene (12-5139); anti-LILRB5, R&D Systems (AF3065); anti-PirB-PE, R&D Systems (FAB2754P); anti-human LAIR1-PE, BD Pharmingen (550811); anti-mouse LAIR1-PE, eBioscience (12-3051); anti-FLAG-APC, Prozyme (PJ255); anti-pCAMKII, Abcam (ab32678); anti-pCAMKIV, Santa Cruz (sc-28443-R); anti-CAMKII, Cell Signaling (4436); anti-CAMKIV, Cell Signaling (4032); anti-Angptl-5, Abcam (ab57240); anti-PirB, BD Pharmingen (550348) for co-IP of PirB; anti-SHP-2, Cell Signaling (3397S) for co-IP of SHP-2; and anti-hFc, Jackson ImmunoResearch (109-036-098). Combinations of multiple lentivirus-expressed shRNAs for inhibition of LILRB2 (hairpin sequences: SEQ ID NO: 20 and SEQ ID NO: 21), Angptl-1 (SEQ ID NO: 22, SEQ ID NO: 23, and SEQ ID NO: 24), Angptl-2 (SEQ ID NO: 25, SEQ ID NO: 26, and SEQ ID NO: 27), Angptl3 (SEQ ID NO: 28, SEQ ID NO: 29, and SEQ ID NO: 30), Angptl4 (SEQ ID NO: 31, SEQ ID NO: 32, and SEQ ID NO: 33), Angptl-5 (SEQ ID NO: 34, SEQ ID NO: 35, and SEQ ID NO: 36), Angptl-6 (SEQ ID NO: 37, SEQ ID NO: 38, and SEQ ID NO: 39), and Angptl-7 (SEQ ID NO: 40, SEQ ID NO: 41, and SEQ ID NO: 42) were purchased from Open Biosystems and used for knockdown experiments. The specificity of LILRB2 mAb is confirmed by comparison of binding to all tested LILRA/Bs on transfected 293T cells. The specificities of other anti-LILRBs, anti-PirB, and anti-LAIR1 were confirmed by staining the respective cDNA overexpressed 293T cells.

Co-immunoprecipitation. For in vivo co-IP, 293T cells were transiently co-transfected with plasmids encoding LILRB2-ECD-hFc, PirB-ECD-hFc, or Tie-2-ECD-hFc and FLAG-tagged Angptl-2 or untagged Angptl-5. Protein A beads were added to conditioned medium and collected at 48 hours after transfection, and proteins were detected by anti-FLAG or anti-Angptl-5 by Western blot. For in vitro co-IP, purified Angptl-2-FLAG or GST-Angptl-5 was incubated with purified LILRB2-ECD-hFc or Tie2-ECD-hFc in PBS with 0.1% BSA and 0.1% NP-40 for 2 hours followed by immunoprecipitation with protein A beads and Western blotting.

Liquid-phase binding Assay. Specific binding of radiolabeled GST-Angptl-5 to BAF3 cells stably infected with MSCV-LILRB2-IRES-GFP (as LILRB2-BAF3 cells) was performed by incubating 6×10⁶ LILRB2-BAF3 cells with ¹²⁵I-GST-Angpt1-5 (0.1-100 nM) in 200 μl PBS/1% BSA for 3 hours at 25° C. Non-specific binding on normal BAF3 cells was subtracted. In the competition assay, 2.5×10⁶ LILRB2-BAF3 or BAF3 cells were incubated with unlabeled GST-Angptl-5 (0.1-100 nM) in 200 μl PBS/1% BSA for 1 hour at 25° C., followed by addition of 5 nM of ¹²⁵I-GST-Angptl-5 for a 4-hour incubation. After incubation, the cells were washed twice by centrifugation, resuspended in ice-cold PBS with 1% BSA, and then measured in a scintillation counter.

Cell culture and infection. BAF3 cells were grown in RPMI medium 1640 with 10% FBS and 10% WEHI conditioned cell medium. Human embryonic kidney 293T cells were grown in DMEM with 10% FBS.

For mouse HSC culture, the indicated numbers of BM Lin⁻Sca-1 ⁺Kit⁺CD34⁻Flk-2⁻ cells or fetal liver Lin⁻Sca-1 ⁺Kit⁺ cells isolated from 8-10 week old C57BL/6 CD45.2 mice were plated in one well of a U-bottom 96-well plate (Corning) with 200 μl of the indicated medium. The cells were cultured at 37° C. in 5% CO? with indicated levels of O₂. For the purpose of competitive transplantation, the cells were pooled from 12 culture wells and mixed with competitor/supportive cells before the indicated numbers of cells were transplanted into each mouse. For Western blotting, 3-week old mouse spleen cells were cultured overnight in DME supplemented with 0.1% BSA, followed by treatment with indicated amount of Angptls. Human mononuclear cord blood cells were cultured in DME containing 10% FBS overnight, followed by starvation in serum-free DME for 4 hours before Angptl stimulation.

The infection of Lin⁻ cells by MSCV-MLL-AF9-IRES-YFP and MSCV-AML1-ETO9a-IRES-GFP was performed using the following method. Lin⁻ cells were incubated overnight in medium with 10% FBS, 20 ng/mL SCF, 20 ng/ml IL-3, and 10 ng/mL IL-6, followed by spin infection with retroviral supernatant in the presence of 4 μg/mL polybrene. Infected cells (300,000 cells) were transplanted into lethally irradiated (1000 rad) C57BL/6 mice by retro-orbital injection.

For human cell culture, fresh and cryopreserved human cord blood cells were obtained from UT Southwestern Parkland Hospital. CD34⁺ cells were isolated by AutoMACS, and cultured essentially as described in Zhang et al. (Blood 111:3415-3423, 2008). CD133⁺ cells were purchased from AllCell Inc. Lentiviral infection by shRNAs for LTLRB2 or Angptls was performed as recommended by Open Biosystems.

Flow cytometry and reconstitution analysis. Donor mouse bone marrow cells were isolated from 8-10 week old C57BL/6 CD45.2 mice. BM Lin⁻Sca-1 ⁺Kit⁺CD34⁻Flk-2⁻ cells were isolated by staining with a biotinylated lineage cocktail (anti-CD3, anti-CDS, anti-B220, anti-Mac-1, anti-Gr-1, anti-Ter119, and anti-7-4; Stem Cell Technologies) followed by staining with streptavidin-PE/Cy5.5, anti Sca 1 FITC, anti-Kit-APC, anti-CD34-PE, and anti-Flk-2-PE. The indicated numbers of mouse CD45.2 donor cells were mixed with 1×10⁵ freshly isolated CD45.1 competitor bone marrow cells, and the mixture injected intravenously via the retro-orbital route into each of a group of 6-9 week old CD45.1 mice previously irradiated with a total dose of 10 Gy. To measure reconstitution of transplanted mice, peripheral blood was collected at the indicated times post-transplantation, and CD45.1⁺ and CD45.2⁺ cells in lymphoid and myeloid compartments were measured. The analyses of Mac-1, Kit, Gr-1, CD3, and B220 populations in AML blood or bone marrow were performed by using anti-Mac-1-APC, anti-Kit-PE, anti-Gr-1-PE, anti-CD3-APC, and anti-B220-PE.

Uncultured or cultured progenies of human cells were pooled together and the indicated portions were injected intravenously via the retro-orbital route into sub-lethally irradiated (250 rad) 6-8 week old NOD/SCID mice. Eight weeks after transplantation, bone marrow nucleated cells from transplanted animals were analyzed by flow cytometry for the presence of human cells.

CFU assays. Two thousand YFP⁺Mac-1 ⁺Kit⁺ BM cells from AML mice were plated in methylcellulose (M3534, Stem Cell Technologies) for CFU-GM assays, according to the manufacturer's protocols. After 7 days, 2000 cells from initially plated three dishes were used for secondary replating.

Surface plasmon resonance. Biacore 2000 and CMS chips were used to analyze binding of purified Angptls to the LILRB2 extracellular domain fused to hFc. Recombinant protein A (Pierce) was pre-immobilized in two flow cells (˜2,000 RU) using the amine-coupling kit from GE. LILRB2-hFc was injected into one of the flow cells to be captured by the protein A to reach ˜300 response units (RU). GST-Angptl-5 was injected over the immobilized LILRB2 in HBS-EP (GE) containing 0.01 M HEPES (pH 7.4), 0.15 M NaCl, and 0.005% polysorbate 20. Each binding sensorgram from the sample flow cell, containing a captured LILRB2-hFc, was corrected for the protein A coupled cell control. Following each injection of an antigen solution, which induced the binding reaction, and the dissociation period during which the running buffer was infused, the protein A surface was regenerated by the injection of the regeneration solution containing 10 mM Na₃PO₄ (pH 2.5) and 500 mM NaCl. All captured LILRB2-hFc, with and without Angptl-5 bound, was completely removed, and another cycle begun. All measurements were performed at 25° C. with a flow rate of 30 μL/min.

GSEA analysis. Gene set enrichment analysis was performed using GSEA v2.0 software (see, Broad Institute website) with 1,000 phenotype permutations, and normalized enrichment score (NES) and false discovery rate q-value (FDR q-val) were calculated.

### Results

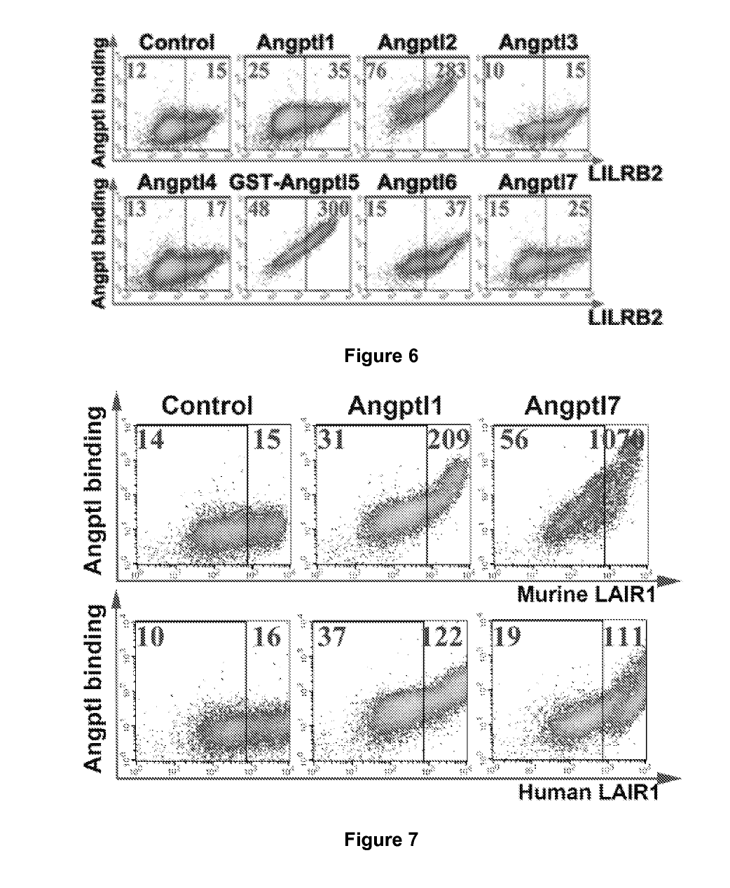

Human LILRB2, when ectopically expressed on BAF3 cells, enables cells to specifically bind GST-Angptl-5 as determined by flow cytometry. LILRB2 is a member of the immune inhibitory B-type subfamily of LILR receptors (Barrow et al., Immunol. Rev. 224:98-123, 2008) and contains four Ig-domains and three immunoreceptor tyrosine-based inhibitory motifs. Flow cytometry data show that LILRB2-overexpressing 293T cells have enhanced binding to several Angptls, especially Angptl-2 and GST-Angptl-5 (FIG. 6). Angptl-2 and GST-Angptl-5 also bound to LILRB3- and LILRB5-overexpressing cells, though with a lower affinity than to LILRB2-expressing cells (Table 1). In addition, Angptl-1 and Angptl-7 bound to LAIR1-overexpressing 293T cells (Table 1 and FIG. 7). Angptls did not bind to LILRAs, LILRB1, or LILRB4 (Table 1).

TABLE 1
Summary of Angptl binding to various Ig-domain containing receptors as determined using
flow cytometry¹

| | Angptl1 | Angptl2 | Angptl3 | Angptl4 | GST-Angptl5 | Angptl6 | Angptl7 |
|---|---|---|---|---|---|---|---|
| LILRA1 | − | − | − | − | − | − | − |
| LILRA2 | − | − | − | − | − | − | − |
| LILRA4 | − | − | − | − | − | − | − |
| LILRB1 | − | − | − | − | − | − | − |
| LILRB2 | + | ++ | +/− | +/− | +++ | +/− | + |
| LILRB3 | − | + | − | − | + | − | − |
| LILRB4 | − | − | − | − | − | − | − |
| LILRB5 | − | +/− | − | − | + | − | − |
| Lair-5 | + | +/− | +/− | − | +/− | +/− | + |

¹The results were summarized from binding to LILRB2-transfected 293T cells and human cord blood LILRB2⁺ cells. GST-Angptl-1, -2, -3, -5, -6, and -7 all bind to human cord blood LILRB2⁺ cells.

As Angptl-2 and GST-Angptl-5 bound to LILRB2-expressing cells better than did other Angptls, additional experiments were performed to assess the molecular interaction between Angptl-2/Angptl-5 and LILRB2. Co-transfection of Angptl-2 or Angptl-5 with LILRB2 extracellular domain (ECD) fused to human IgG-Fc (LILRB2-hFc) into 293T cells followed by immunoprecipitation (IP)/Western blot revealed that both Angptl-2 and Angplt-5 interacted with the extracellular domain of LILRB2, but not that of Tie-2. The direct interactions between Angptls and LILRB2 were confirmed by in vitro co-IP using purified Angptl-2-FLAG or GST-Angptl-5 and LILRB2-hFc and by surface plasmon resonance (SPR). A liquid-phase binding assay with ¹²⁵I-labeled GST-Angptl-5 demonstrated that the interaction between Angptl-5 and cell surface LILRB2 was specific and saturable, with half maximal saturation of the interaction as 5.5±1.1 nM. While untagged Angptls bind to LILRB2, the type or the position of tagging could affect the binding (Table 2).

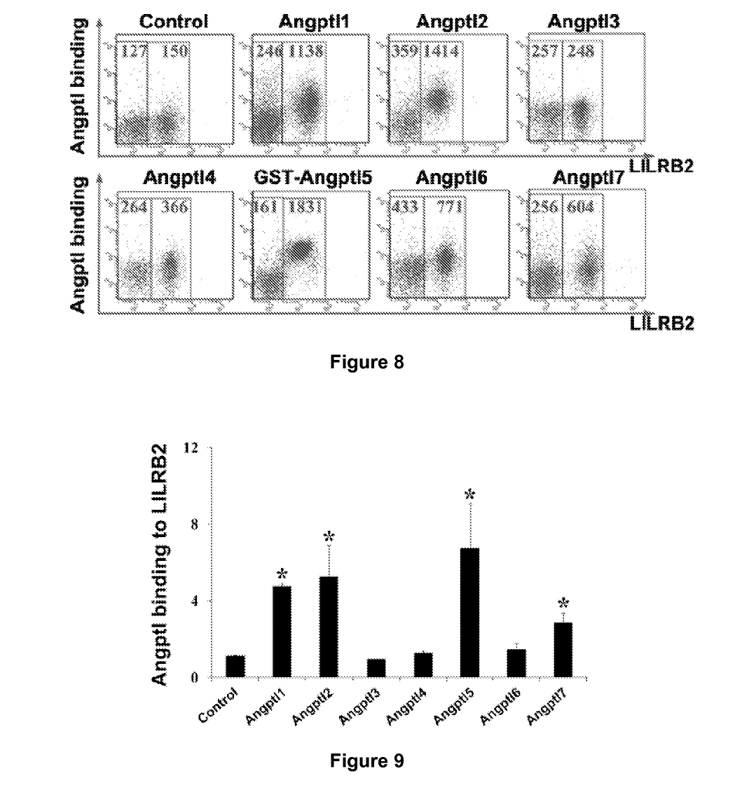

An additional set of experiments was performed in order to determine whether Angptls bound to LILRB2 or LAIR1 on primary human cord blood cells. Flow cytometry analysis showed that Angptl-1, -2, -5, and -7 all bound to LILRB2+ human cord blood cells; with Angptl-2 and GST-Angptl-5 having higher affinities (FIGS. 8 and 9, Table 1). Angptl-1 and Angptl-7 bind to LAIR1+ human cord blood cells relatively weakly. The next set of experiments therefore focused on studying the binding of Angptl-2 and Angptl-5 to LILRB2.

TABLE 2
Summary of the binding of differently tagged Angptl2 and Angptl5 to LILRB2¹

| | A2 | | | | | | A5 | | | |
|---|---|---|---|---|---|---|---|---|---|---|
| | In house | | | | | | In house | | | |
| | (mammalian | | | | A2-CC- | A5-CC- | (mammalian) | | | A5-His |
| | could not be | A2-Flag | | | A5-FBN | A2-FBN | could not be | | | Gift |
| Name | purified in | In house | Flag-A2 | A2-Flag | In house | In house | purified; in | GST- | GST-A5 | from |
| Manu- | vivo CO-IP | (mam- | In house | In house | (mam- | (mam- | vivo CO-IP | A5 | In house | RAD |
| facturer | only) | malian) | (bacterial) | (bacterial) | malian) | malian) | only) | Abnova | (bacterial) | Systems |
| Binding | ++ | ++ | +/− | +/− | ++ | ++ | ++ | +++ | +++ | +/− |
| to | | | | | | | | | | |
| LILRB2 | | | | | | | | | | |

¹A2 or A5: untagged Angptl-2 or Angptl-5
A2 FLAG: FLAG tagged at the C-terminus of Angptl-2, mammalian-expressed (mammalian) or bacterially-expressed (bacterial)
A2-CC-A5-FBN: the coiled-coil domain of Angptl-2 fused to the fibrinogen-like domain of Angptl-5
A5-CC-A2-FBN: the coiled-coil domain of Angptl-5 fused to the fibrinogen-like domain of Angptl-2
GST-A5-FLAG: GST tagged at the N-terminus and FLAG-tagged at the C-terminus of Angptl-5

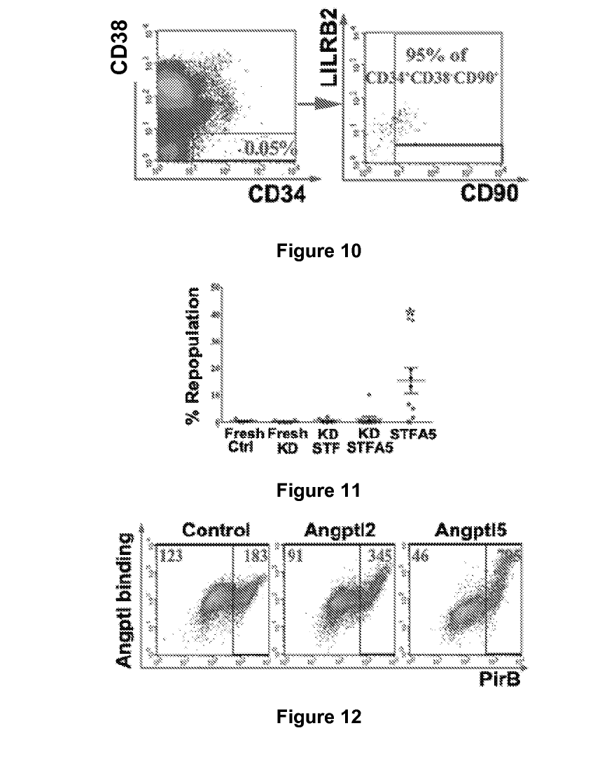

First, a set of experiments was performed to determine whether LILRB2 was expressed on human HSCs. Flow cytometry and real-time RT-PCR analyses revealed that LILRB2 was expressed on the surface of 40-95% of human cord blood CD34⁺CD38⁻CD90⁺ cells (95% in FIG. 10): this population was enriched for HSCs. GST-Angptl-5-treatment resulted in an increase in the phosphorylation of calcium/calmodulin-dependent protein kinase CAMKII and CAMKIV in human cord blood mononuclear cells. CAMKIV is required for maintcnance of the potency of HSCs (Kitsos et al., J. Biol. Chem. 280:33101-33108, 2005). In addition, suppression of LILRB2 expression with shRNAs effectively reduced Angpt-1 binding. Importantly, the silencing of LILRB2 resulted in decreased repopulation of human cord blood HSCs as measured by reconstitution analysis in NOD/SCID mice (1% repopulation from cultured knockdown cells compared to 17% repopulation from cultured normal cells in medium STFAS; FIG. 11). Together, these data indicate that the Angptl-5 supports expansion of human cord blood HSCs in a process at least partially mediated by the surface receptor LILRB2.

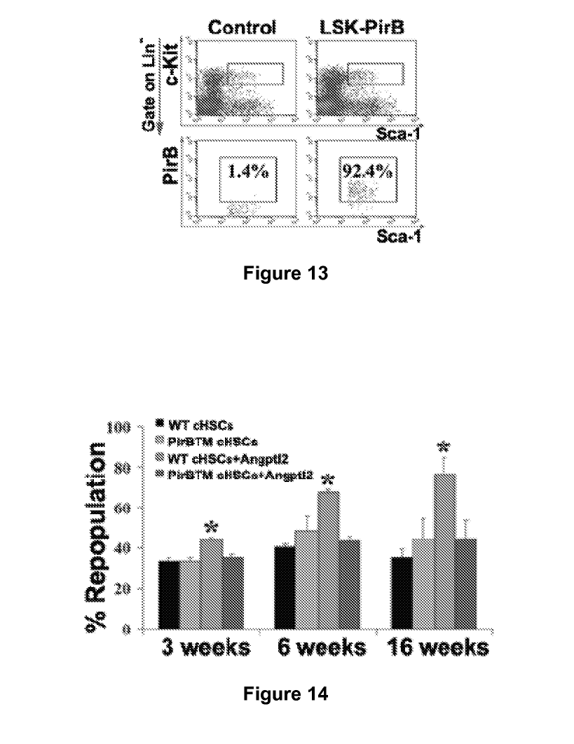

The paired immunoglobulin-like receptor B (PirB) is the mouse membrane ortholog of human LILRBs. Angptl-2, Angptl-3, and GST-Angptl-5 bound to PirB as determined by flow cytometry (FIG. 12) and by Co-IP (FIG. 13). As observed for human cord blood HSCs, mouse HSCs were also enriched for PirB expression.

In order to study the function of PirB in mouse HSCs, experiments were performed using HSCs from PirB-deficient (PirBTM) mice (Syken et al., Science 313:1795-1800, 2006), in which four exons encoding the transmembrane domain and part of the intracellular domain were deleted. Freshly isolated PirBTM HSCs from 3-week old mice had significantly decreased CAMKIV phosphorylation, and binding of Angpt-1 to PirB-induced phosphorylation of PirB, recruitment of SHP-1 and SHP-2, and CAMKIV activation. These results suggest that certain Angptls may be the ligands of PirB that activate CAMKIV in vivo.

Because SHP-2 and CAMKIV are required for the repopulation of HSCs (Kitsos et al., J. Biol. Chem. 280:33101-33108, 2005), and the chemical inhibition of CAMKII, a homolog of CAMKIV, induces differentiation and suppresses proliferation of myeloid leukemia cells (Si et al., Cancer Res. 68:3733-3743, 2008), experiments were performed to determine whether PirB was important for HSC activity. While the adult PirBTM mice have certain immune and neuronal defects, they are grossly normal in hematopoiesis. Interestingly, competitive repopulation showed that PirBTM fetal liver HSCs had approximately 50% decreased repopulation activity. Moreover, although Angptl-2 and Angptl-5 had little effect on ex vivo expansion of adult PirBTM HSCs, they supported ex vivo expansion of adult wild-type (WT) HSCs (FIG. 14). Collectively, these data indicate that Angptls bind human LILRB-2 and mouse PirB to support HSC repopulation.

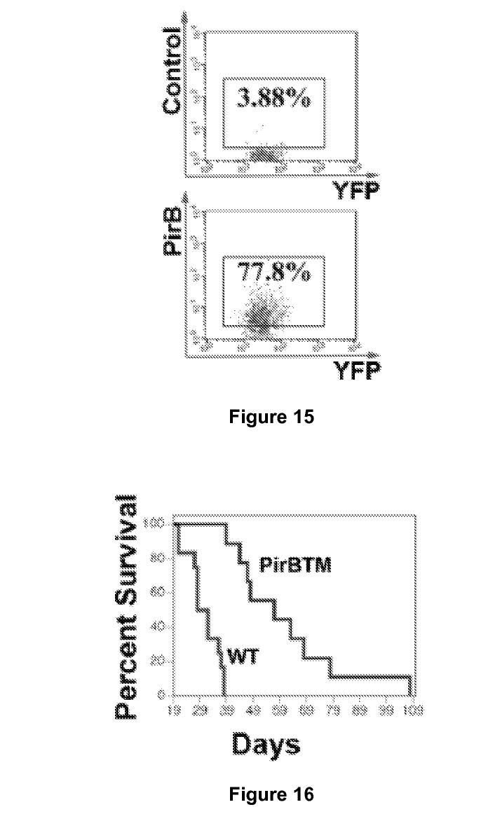

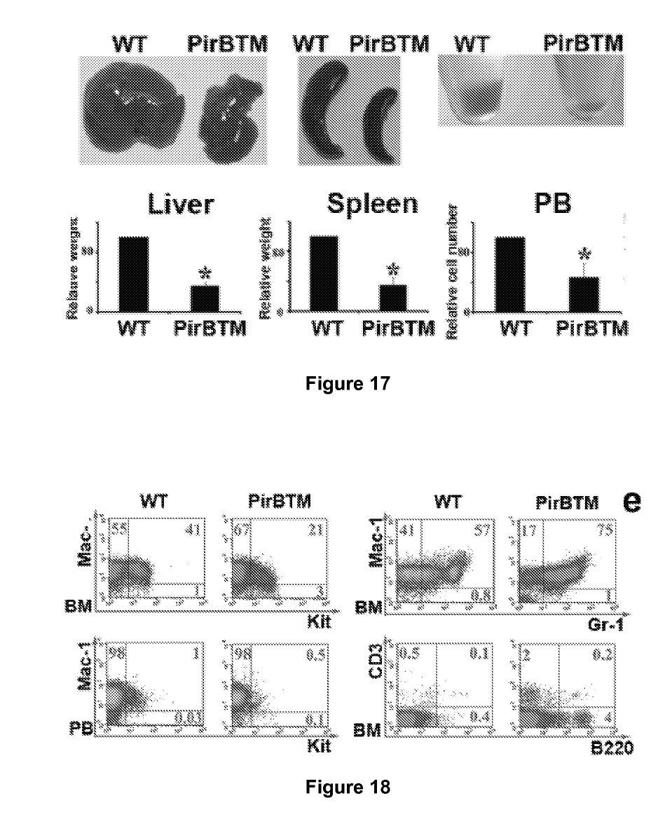

Based on our in silico analysis of a pool of 9004 samples described previously (Lukk et al., Nat. Biotechnol. 28:322-324, 2010), the level of LILRB2 mRNA is at least 4-fold higher in the human acute monoblastic and monocytic leukemia cells (M5 subtype of acute myeloid leukemia (AML)) than in other AML cells. Since human acute monoblastic and monocytic leukemia cells arc often associated with rearrangement of MLL (a histone methyltransferase deemed a positive global regulator of gene transcription), a retroviral MLL-AF9 transplantation mouse model (Krivtsov et al., Nature 442:818-822, 2006) was used to further examine the role of PirB in regulation of AML development. WT or PirBTM donor Lin⁻ cells infected by retroviral MLL-AF9-IRES-YFP were used to induce AML as previously described (Krivtsov et al., supra). The PirB expression was examined in YFP¹Mac-1 ¹Kit¹ cells that may be enriched for AML initiating activity. The data show that about 80% YFP⁺Mac-1 ⁺Kit⁺ cells were PirB⁺ (FIG. 15). Another set of experiments was performed to determine whether PirB was required for the induction of AML by MLL-AF9. Mice transplanted with MLL-AF9-transduced WT cells developed AML and died within approximately 5 weeks, whereas those transplanted with MLL-AF9-transduced PirBTM cells were resistant to the induction of MLL-AF9 and developed AML much more slowly (FIG. 16). The significantly delayed development of the PirBTM leukemia was correlated with about 50% lower numbers of white blood cells in circulation and a much less severe infiltration of myeloid leukemia cells into the liver and spleen (FIGS. 17 and 18). Consistently, PirB deficiency caused an approximately 50% reduction of YFP⁺Mac-1 ⁺kit⁺ cells in both bone marrow and peripheral blood (FIG. 29). There were more CD3⁺ or B220⁺ cells in mice that received MLL-AF9-transduced PirBTM donor cells than in those given WT cells (FIG. 18). These data demonstrate that PirB-mediated signaling is associated with faster AML development and greater numbers of YFP⁺Mac-1 ⁺Kit⁺ AML cells in vivo.

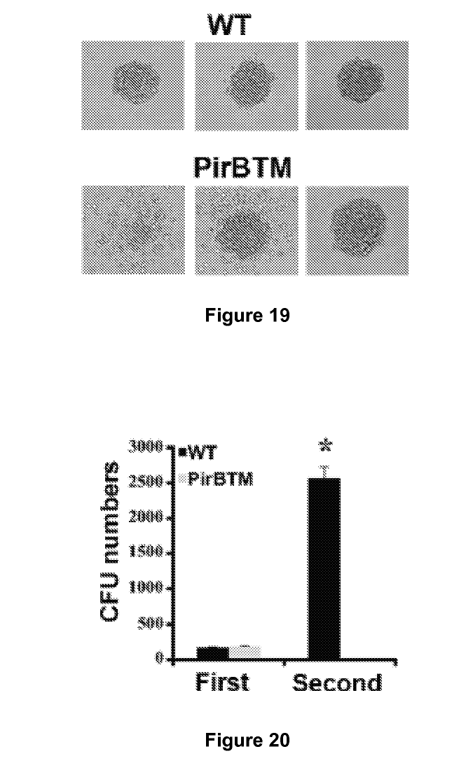

An additional set of experiments was performed to determine whether PirB potentially regulates differentiation and self-renewal of AML cells. CFU assays showed that extrinsic Angptl stimulation led to increased CFU numbers in WT, but not PirBTM AML cells, again indicating PirB directly mediates Angptls' effects. In addition, WT AML cells formed mostly compact colonies, whereas PirBTM cells tended to form more diffuse ones (FIG. 19). The formation of diffuse colonies indicates high differentiation potential. Moreover, PirBTM primary CFUs were unable to form secondary colonies upon replating (FIG. 20), suggesting that PirB supports self-renewal of AML CFU cells.

The molecular signaling triggered by the binding of Angptls to PirB in AML cells was also investigated. PirBTM AML cells had decreased phosphorylation of phosphatase SHP-2, which is known to be associated with LILRB receptors and is an oncogene that supports leukemia development. Angptls also stimulated SHP-2 phosphorylation. Similar to untransformed PirBTM cells, PirBTM AML cells had decreased CAMKIV activation. Furthermore, WT Mac-1 ⁺kit⁺ cells had much greater expression of leukemia initiation/maintenance genes, but dramatically decreased expression of myeloid differentiation genes as determined by DNA microarray analyses. Quantative RT-PCR confirmed the increased expression of several HoxA genes, Meis1, Eya1, Myb, and Mef2c in WT Mac-1 ⁺Kit⁺ cells than PirBTM counterparts; these genes are critical for initiation or maintenance of MLL rearranged AML. Similar to the MLL-AF9 model, the deficiency of PirB in the AML1-ETO9a leukemia model led to decreased leukemia progenitors and increased differentiated cells. Collectively, these results suggest that the binding of Angptls to PirB promotes leukemia development, likely through inhibiting differentiation of AML cells.

### Example 3

### GA Competes for Binding to LILRB2 and LILRB4 with Anti-LILRB2 and Anti-LILRB4 Antibodies

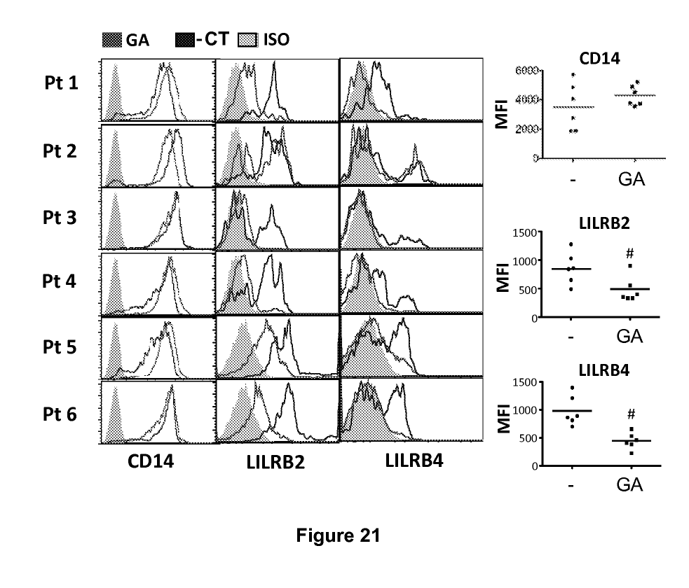

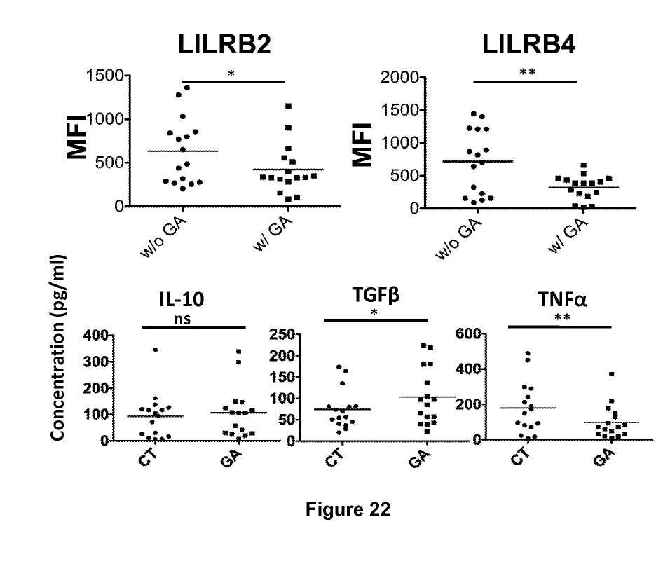

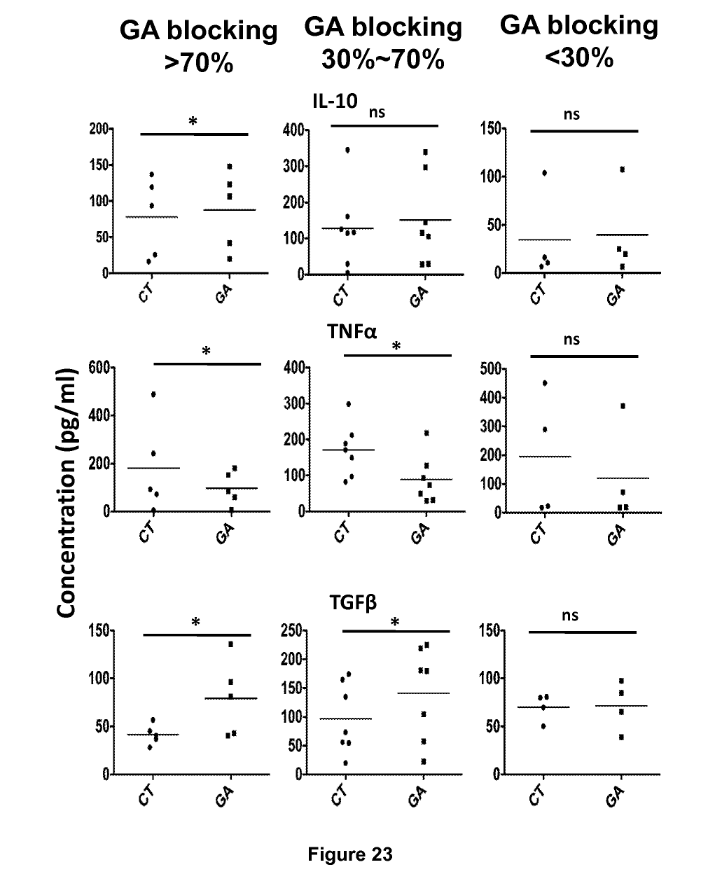

A set of experiments were performed to determine whether LILRB2 and LILRB4 are expressed on the surface of human MDSCs from cancer patients. The resulting data show that LILRB2 and LILRB4 are expressed on the surface of human MDSCs (FIG. 21). GA treatment resulted in increased cytokine expression in these cells (FIG. 22). Competitive binding assays also showed that, in the presence of excess GA, the binding of specific antibodies to LILRB2 and LILRB4 on MDSCs from different patients was significantly reduced as evidenced by decreased mean fluorescence intensities (FIG. 23). This decrease was more extensive in MDSCs expressing high levels of LILRB2 and LILRB4 than in those expressing low levels of LILRB2 and LILRB4 (left panel, FIG. 23).

### Example 4

### Effects of LILRB2-4, Lair1, SHP-1, and CAMKs on Growth of Human Leukemia Cells

A set of experiments was performed to test the effects of LILRB2, LILRB3, LILRB4, Lair1, SHP-1, and CAMKs on growth of human leukemia cells. A summary of the experimental protocols is provided below.

### Methods

The knockdown of expression of individual LILRBs was performed by introducing lentivirus encoded small hairpin RNAs (shRNAs) into a number of different human leukemia cell lines: MV4-11 (AML), 697 (B-ALL), and RCH-ACV (B-ALL). The lentiviral vector PII3.7 was used to express shRNAs designed to target LILRBs.

The mRNA expression data for LILRB1-4 in AML patients was determined using the TCGA AML database (tcga-data.nci.nih.gov website), and normalized by GADPH expression. The data was compared by separating patients into two groups based on their higher or lower expression levels than the average (GADPH-normalized) expression levels of the indicated genes. Survival curves of patients with higher or lower expression levels of the indicated genes were generated.

The expression levels of many ITIM-containing receptors were inversely correlated with survival in AML patients. In these experiments, in silico analysis of the relationship between 54 human ITIM-receptor mRNA and AML patient survival was performed. The expression data were obtained from the TCGA AML database and were normalized by GADPH expression, Affymetrix housekeeping gene normalization, or total normalization by setting the average expression on each chip to 1,000 to avoid batch effects. The patents were separated into two groups based on whether they have higher (n=82) or lower (n=83) than the average (normalized) expression levels of the indicated genes (n=165).

ITIM receptor knockdown in MV4-11 and NB4 AML cells was performed by shRNA (using lentivirus infection as generally described herein). In these experiments, the cell number was calculated on day 6 post-infection (n=3). Similar knockout experiments were performed to knockdown expression of lair1 in MV4-11 cells.

Additional studies were performed to determine whether lair1 knockdown would block xenograft of human leukemia cell lines. In these experiments, MV4-11 cells (1×10⁶ total cells) were infected with virus designed to express GFP and either scrambled shRNA or shRNA targeting lair1. The cells were collected and transplanted into NSG mice (n=7). The relative percentages of GFP⁺ cells in BM, spleen, liver, and perpherial blood were determined one month after transplantation.

Additional experiments were performed to study the effect of lair knockdown on primary human AML development. In these experiments, GFP primary human AML cells (5×10⁶ cells) infected by scrambled shRNA- or lair-1 shRNA-lentivirus were collected and transplanted into NSG mice. The survival curves of the mice receiving control or lair-1 knockdown primary human AML cells (sample #6) were determined The percentages of GFP cells in bone marrow of NSG mice transplanted with control or lair1-knockdown primary human AML cells at 4 months after transplantation were also assessed.

The expression of SHP-1 was assessed in patients having AML. These data were obtained from the TCGA AML database. The patients were separated into two groups based on whether they have higher (n=93) or lower (n=93) than the average expression levels of shp-1 (n=186).

Additional experiments were performed to knockdown shp-1 expression in MV4-11 cells. Three different shRNAs were used to target shp-1. Apoptosis of MV4-11 cells was assessed at different time points after shRNA (shRNA 298) infection. The apoptotic cells were quantitated using flow cytometry.

In another set of experiments, survival curves were generated for mice receiving 3,000 cre- or control-infected YFP¹ BM cells that were collected from primary recipient mice transplanted with SHP-1 knockout (Cre) or control (Ctrl) MLL-AF9 AML cells. The percentages of GFP AML cells in the peripheral blood in these mice were also determined. The colony-forming activity of the shp-1 knockout (cre) and control MLL-AF9 AML cells was also assessed. An additional experiment was performed to determine the effect of the SHP-1 inhibitor sodium stibogluconate on the colony forming activity of MLL-AF9 AML cells.

The CAMK expression in AML patients was assessed using the mRNA expression data from the TCGA AML database. The CAMK expression data was normalized by GADPH expression. The patients were separated into two groups based on whether they have higher (n=93) or lower (n=93) than the average expression levels of the indicated genes (n=186). Survival curves for the two groups of patients were generated.

Additional experiments were performed to knockdown CaMKI and CaMKIV in human B-ALL U937 cells and human AML MV4-11 cells (using lentivirus encoding shRNA targeting CaMKI or CaMKIV). The colony forming activity of WT and PirBTM AML cells following treatment with a CaMKK inhibitor (ST0609) or a CaMK inhibitor (KN93) was also assessed.

### Results

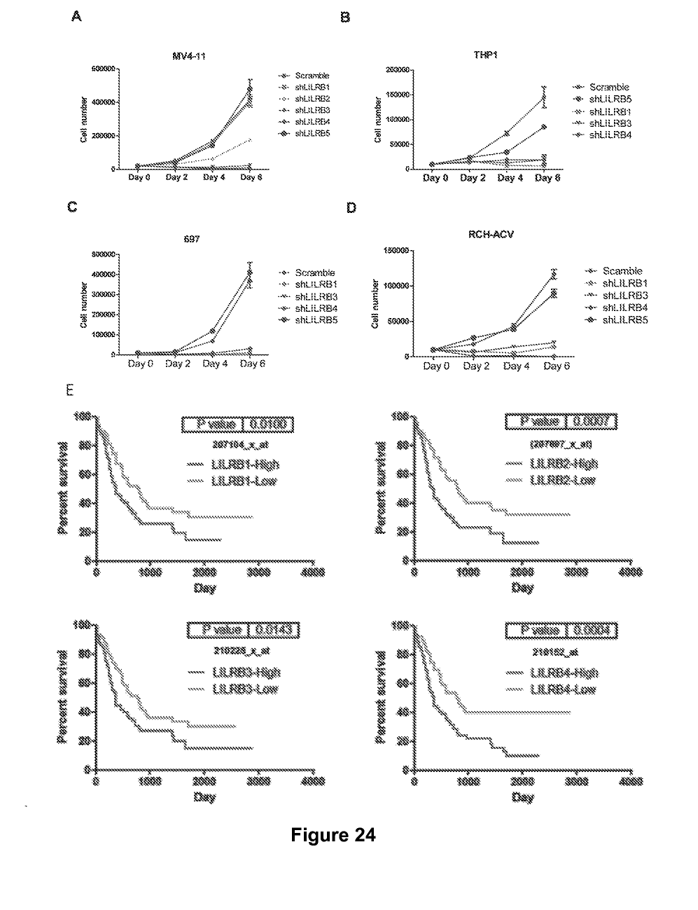

The data indicate that knockdown of LILRB2, LILRB3, or LILRB4 decreases the growth of human leukemia cells lines (FIG. 24A-D). In addition, the expression level of each of LILIRB1, LILRB2, LILRB3, or LILRB4 negatively correlates with overall survival of AML patients (FIG. 24E). These data suggest that methods of decreasing LILRB1, LILRB2, LILRB3, or LILRB4 activity or expression levels would decrease the growth of leukemia cells in a mammal, and could be used to treat leukemia in a mammal.

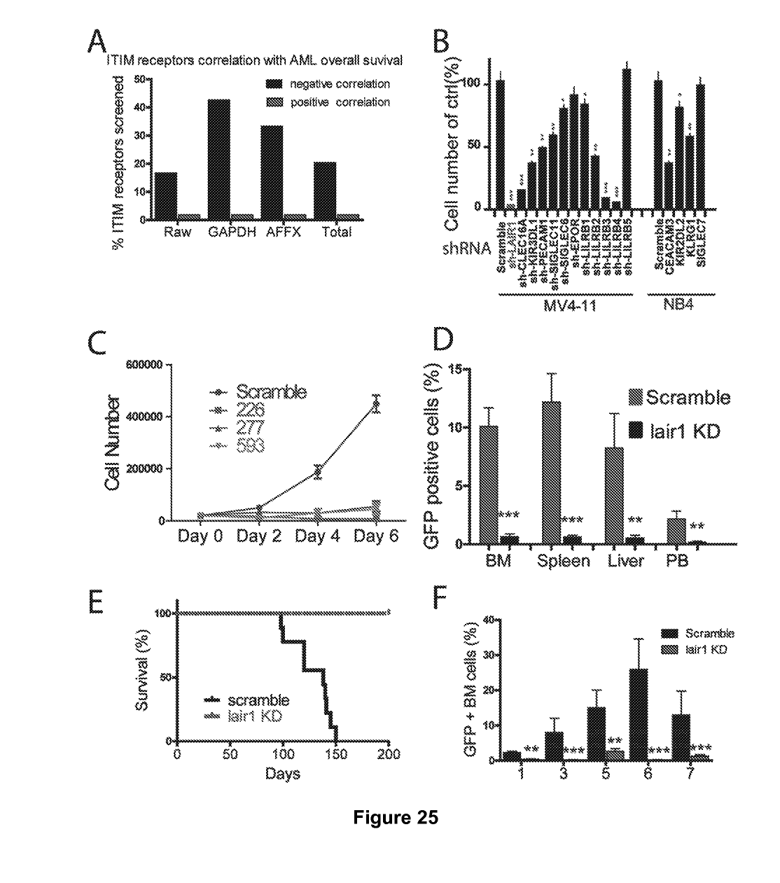

The expression of a number of ITIM-containing receptors also inversely correlated with survival of AML patients (FIG. 25A). The expression of several ITIM-receptors was shown to negatively correlate with overall survival of patients: CD22, CD300A, CD300LF, CD72, CEACAMM1, CEACAM3, CLEC16A, KIR2DL3, KLRG1, LILRB1, LILRB2, LILRB3, LILRB4, NCR2, PECAM1, PTPRO, RTN4, SIGLEC11, SIGLEC6, SIGLEC7, and SIGLECL1. The knockdown of several ITIM-receptors was shown to decrease the growth of leukemia cells (FIG. 25B). For example, the knockdown of Lair1, LILRB2, LILRB3, LILRB4, CLEC16A, KIR3DL1, PECAM1, SIGLEC11, SIGLEC6, CEACAM3, KIR2DL2, or KLRG1 significantly decrease the growth of human leukemia cells (FIG. 25B). The knockdown of Laid significantly inhibited the growth of MV4-11 leukemia cells (FIG. 25C). The knockdown of Lair1 also blocked the xenograft of human leukemia cells lines (FIG. 25D), inhibited primary human AML development (FIG. 25E), and decreased the AML development in the bone marrow in a xenograft model (FIG. 25F). These data indicate that agents that inhibit the expression or activity (e.g., shRNA or oligonucleotides) of Lair1, LILRB2, LILRB3, LILRB4, or other ITIM-containing receptors can decrease leukemia cell growth, and can be used to treat leukemia in a subject.

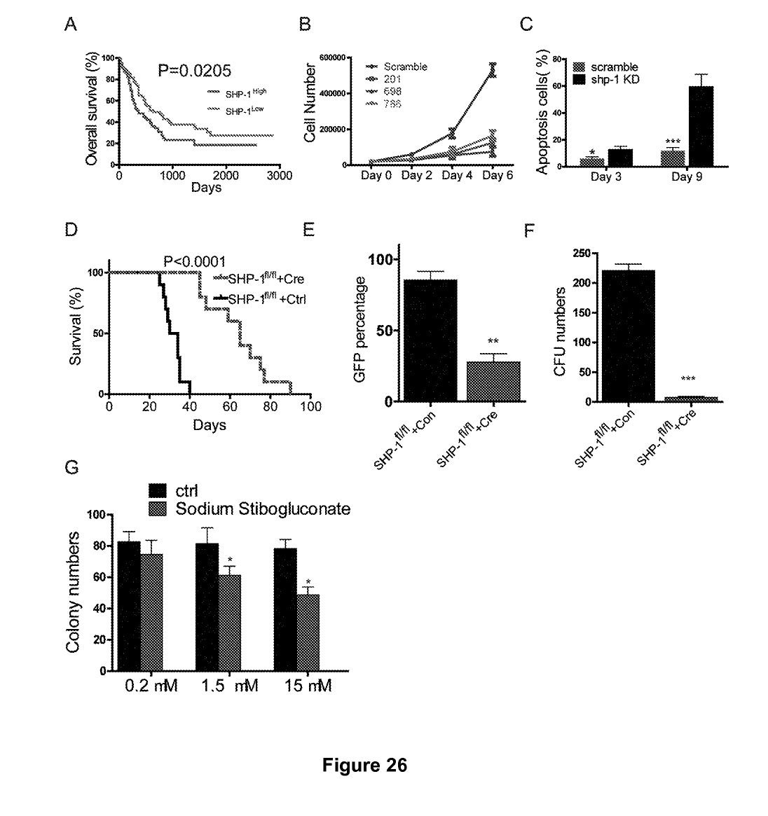

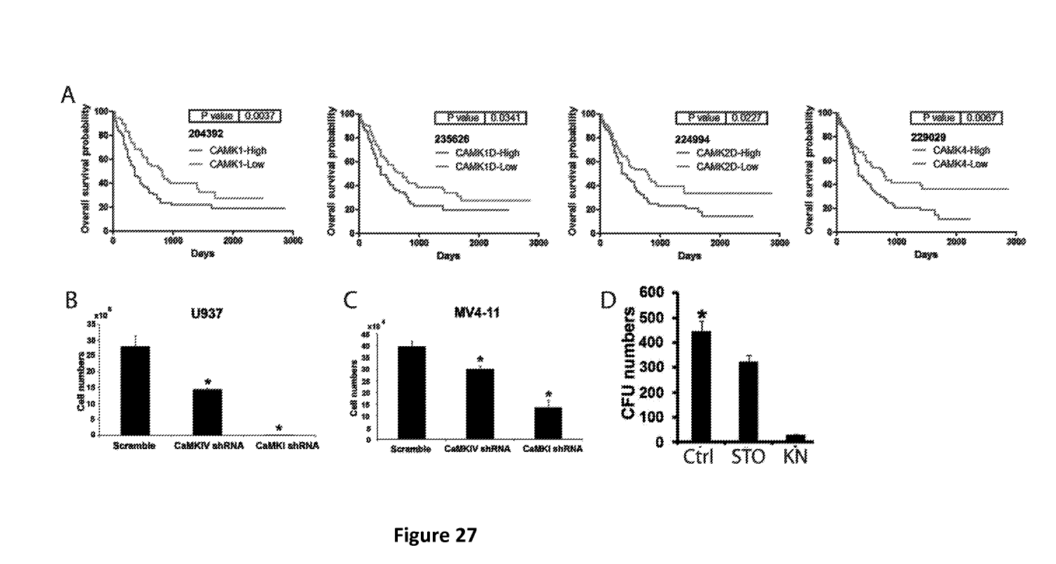

The expression of SHP-1 also negatively correlates with survival in subjects having AML (FIG. 26A). Treatment with an shRNA targeting shp-1 inhibits the growth of a leukemia cell line (FIG. 26B) and increases apoptotic cell death in a leukemia cell line (FIG. 26C). Mice transplanted with shp-1 knockdown AML cells had worse survival than mice transplanted with control AML cells (FIG. 26D). The mice transplanted with the shp-1 knockdown AML cells had decreased AML cells in the peripheral blood (FIG. 26E). The shp-1 knockdown AML cells also had decreased colony-forming activity (FIG. 27F). The administration of a SHP-1 inhibitor (sodium stibogluconate) also decreased the colony forming activity of WT leukemia cells (FIG. 26G). These data indicate that agents that inhibit the expression or activity (e.g., shRNA, oligonucleotides, or sodium stibogluconate) of SHP-1 can decrease leukemia cell growth and can be used to treat leukemia in a subject.

The expression of several different CAM kinases also negatively correlate with survival in AML patients: CAMK1, CAMK1D, CAMK2D, and CAMK4 (FIG. 27A). The knockdown of CaMKI and CaMKIV significantly decreased the growth of human B-ALL U937 cells and human AML MV4-11 cells (FIGS. 27B and 27C, respectively). The CaMKK inhibitor ST0609 and the CaMK inhibitor KN93 decreased the colony forming activity of WT AML cells (FIG. 27D). These data indicate that agents that inhibit the expression or activity (e.g., shRNA, oligonucleotides, or chemical inibitors) of CAMK1, CAMK1D, CAMK2D, CAMK4, or CaMKK can decrease leukemia cell growth, and can be used to treat leukemia in a subject.

### Example 5

### Effect of Angptl-5 and anti-Angptl-5 Antibodies on MDSC Activity

A set of experiments were performed in order to test the effect of Angptl-5 and anti-Angptl-5 antibodies on MDSC activity. A summary of the experimental protocols is provided below.

### Methods

Experiments were performed to determine whether Angptl-5 would affect the secretion of interferon-γ and IL-10 by MDSC. In a first set of experiments, human PBMCs were stimulated with OKT3 (1 μg/mL) and anti-CD28 (1 μg/mL) for 4 days, in the presence or absence of Angptl-5 (10% Angptl-5 transfected culture supernatant or control supernatant), and the PBMC culture supernatant was collected for measurement of interferon-γ using an ELISA. In a second set of experiments, sorted human CD33⁺CD14⁺ cells were stimulated with Angptl5 (10% supernatant) for 48 hours, and the IL-10 and IL-4 levels in the supernatant were measured using ELISA assays. In a third set of experiments, CD33⁺ human MDSCs were purified, and treated with control IgG or anti-LILRB1-4 antibodies at a final concentration of 5 μg/mL for 30 minutes. Five hundred-thousand of the MDSCs were seeded into a 96-well plate coated with 10% supernatant from 293T cells without (untreated) or with transfection of Angptl-5. After 2-day culture, the supernatant was collected, and the production of IL-10 was detected using an ELISA.

A separate set of experiments was performed to test whether anti-LILRB antibodies can enhance MDSC-mediated tumor killing and revert the MDSC-mediated suppression of cytolytic T-cell tumor killing effect. In these experiments, purified CD33⁺ human MDSC were incubated with control IgG, or anti-LILRB antibodies for 48 hours, and used in a direct cytolytic assay with K562 tumor cells (4 hour incubation). In a separate experiment, irradiated, purified CD33⁺ MDSCs were incubated with control IgG, anti-LILRB3 antibodies, or anti-LILRB3 and anti-LILRB4 antibodies for 30 minutes, followed by co-culturing with MDSC-depleted peripheral blood mononuclear cells at a 1:1 ratio in the presence of OKT3 (1 μg/mL) and anti-CD38 antibodies (1 μg/mL) for 4 days. The cells were then harvested and co-cultured with leukemia tumor target U937 cells at a 100:1 ratio for 4 hours. The supernatants were harvested and cytotoxicity was measured using the Cytotox 96 non-radioactive assay kit (Promega).

A set of experiments was performed to determine whether anti-LILRB3 antibodies would inhibit the growth of leukemia in a mammal. In these experiments, immunodeficient SCID mice were inoculated subcutaneously with 5×10⁶ of U937 cells on day 0, and intravenously injected with 100 μg of control IgG or anti-LILRB3 (R&D Systems) every 3 days. The tumor burden was measured and tumor size was calculated every 3 days.

### Results

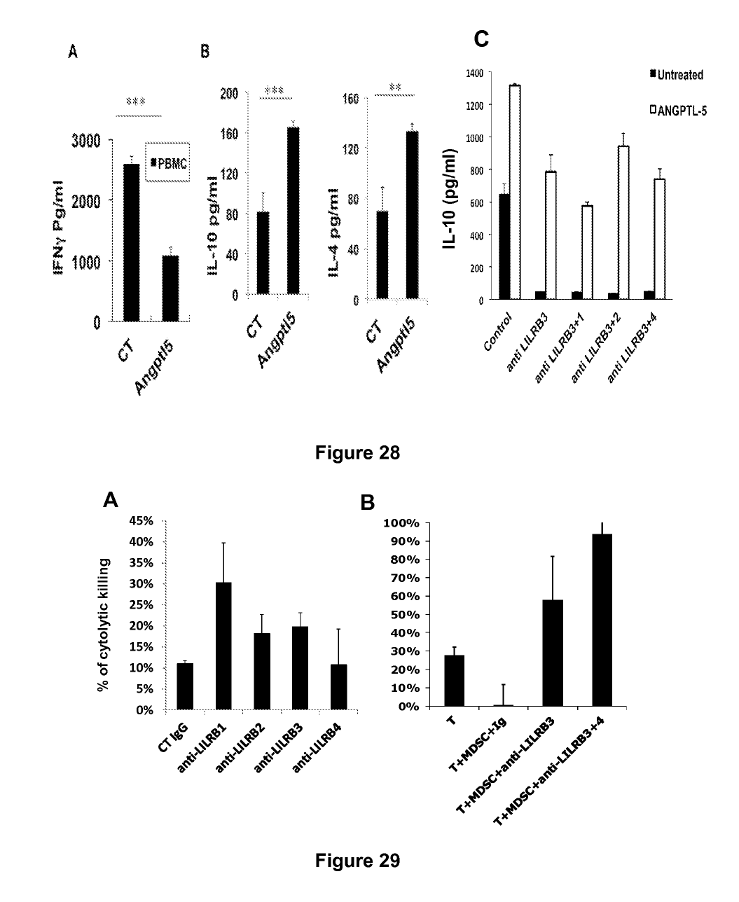

The data show that Angptl-5 can suppress interferon-γ production in human MDSCs stimulated with OKT3 and anti-CD28 (FIG. 28A). Furthermore, Angptl-5 treated human MDSCs secreted significantly higher amounts of IL-4 (FIG. 28B). Angptl-5 can also induce the human MDSCs to secrete significant amounts of IL-10 that can be inhibited by the addition of anti-LILRB antibodies (FIGS. 28B and 28C).

The data further show that the addition of LILRB blocking antibodies, e.g., anti-LILRB1 antibodies, anti-LILRB2 antibodies, and anti-LILRB3 antibodies, can enhance the MDSC-mediated tumor killing (FIG. 29A), and that anti-LILRB3 antibodies alone and the combination of anti-LTLRB3 antibodies and anti-LILRB4 antibodies can reverse (revert) the MDSC-mediated suppression of T cell-mediated cytolytic activity against leukemia cells (FIG. 29B).

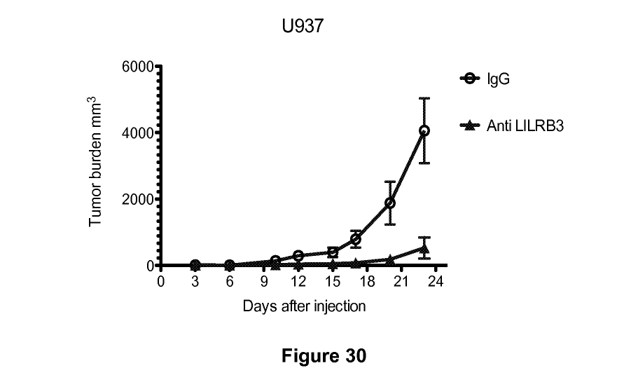

Finally, the data in FIG. 30 show that anti-LILRB3 antibodies can inhibit tumor growth in a leukemia animal model (when compared to IgG-injected (control) mice). These data indicate that anti-LILRB3 antibodies can inhibit tumor growth in vivo, and can be used to treat cancer (e.g., leukemia) in a mammal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-46. (canceled)

47. A method of modulating myeloid-derived suppressor cells (MDSCs) present in a subject, the method comprising:
contacting the MDSCs with an agent that regulates a target protein expressed on the MDSCs, wherein the target protein is selected from the group consisting of leukocyte immunoglobulin-like receptor (LILR) B1 (LILRB1), LILRB2, LILRB3, LILRB4, LILRB5 and LAIR1.

48. The method of claim 47, wherein the MDSCs are CD33⁺.

49. The method of claim 48, wherein the MDSCs are CD11b⁺, or CD14⁺.

50. The method of claim 47, wherein the MDSCs comprise at least one of myeloid progenitors, immature or mature macrophages, immature granulocytes and immature dendritic cells.

51. The method of claim 47, wherein the agent modulates differentiation, polarization, repopulation or depletion of MDSCs.

52. The method of claim 47, wherein the agent is an agonist of the target protein.

53. The method of claim 47, wherein the agent is an inhibitor of the target protein.

54. The method of claim 47, wherein the agent specifically binds to the target protein.

55. The method of claim 47, wherein the agent blocks binding of a ligand to the target protein.

56. The method of claim 47, wherein the agent is an antibody or antigen-binding fragment thereof.

57. The method of claim 56, wherein the antibody or antigen-binding fragment thereof is a monoclonal antibody, a polyclonal antibody, a human antibody, a humanized antibody, a chimeric antibody, a single-chain antibody, a bi-specific antibody, a single-chain Fv (scFv), a Fab fragment, a Fab′ fragment, a F(ab′)2 fragment, an IgG1 antibody, an IgG2 antibody, an IgG3 antibody, or an IgG4 antibody.

58. The method of claim 47, wherein the agent is glatiramer acetate or an angiopoietin-like (Angptl) protein.

59. The method of claim 58, wherein the angiopoietin-like protein is Angptl-1 protein, Angptl-2 protein, Angptl-3 protein, Angptl-4 protein, Angptl-5 protein, Angptl-6 protein or Angptl-7 protein.

60. The method of claim 47, wherein the agent is an oligonucleotide.

61. The method of claim 60, wherein the oligonucleotide is an inhibitory RNA, an antisense RNA or a ribozyme.

62. The method of claim 61, wherein the inhibitory RNA is a small interfering RNA (siRNA).

63. The method of claim 47, wherein the MDSCs are differentiated or polarized into M1 cells.

64. The method of claim 47, wherein the MDSCs are differentiated or polarized into M2 cells.

65. The method of claim 47, wherein the subject is diagnosed as having autoimmune disorder, inflammation or transplant rejection.

66. The method of claim 47, further comprising decreasing MDSC-mediated immune response or increasing MDSC-mediated suppression of immune response.

67. The method of claim 47, wherein the subject is diagnosed as having cancer.

68. The method of claim 67, wherein the cancer is acute myeloid leukemia (AML).

69. The method of claim 67, further comprising increasing MDSC-mediated tumor killing or decreasing MDSC-mediated suppression of tumor killing.

70. The method of claim 47, further comprising increasing MDSC-mediated immune response or decreasing MDSC-mediated suppression of immune response.
